SEQUENCE LISTING (187 sequences)

SEQ ID NO: 1 - protein, 29 aa, Oldenlandia affinis
GLPVCGETCVGGTCNTPGCTCSWPVCTRN

SEQ ID NO: 2 - protein, 29 aa, Oldenlandia affinis
GLPVCGETCFGGTCNTPGCSCTWPICTRD

SEQ ID NO: 3 - protein, 29 aa, Artificial Sequence
  D kalata B2
GLPVCGETCFGGTCNTPGCSCTWPICTRD

SEQ ID NO: 4 - protein, 16 aa, Artificial Sequence
  [G18K]kalata B1
GLPVCGETCVGGTCNT

SEQ ID NO: 5 - protein, 29 aa, Artificial Sequence
  [N29K]kalata B1
GLPVCGETCVGGTCNTPGCTCSWPVCTRK

SEQ ID NO: 6 - protein, 29 aa, Artificial Sequence
  [T20K, G1K]kalata B1
KLPVCGETCVGGTCNTPGCKCSWPVCTRN

SEQ ID NO: 7 - protein, 29 aa, Artificial Sequence
  [T20K]kalata B1
GLPVCGETCVGGTCNTPGCKCSWPVCTRN

SEQ ID NO: 8 - protein, 29 aa, Artificial Sequence
  [T8K]kalata B1
GLPVCGEKCVGGTCNTPGCTCSWPVCTRN

SEQ ID NO: 9 - protein, 29 aa, Artificial Sequence
  [V10A]kalata B1
GLPVCGETCAGGTCNTPGCTCSWPVCTRN

SEQ ID NO: 10 - protein, 29 aa, Artificial Sequence
  [V10K]kalata B1
GLPVCGETCKGGTCNTPGCTCSWPVCTRN

SEQ ID NO: 11 - DNA, 87 bp, Oldenlandia affinis
ggacttccagtatgcggtgagacttgtgttgggggaacttgcaacactccaggctgcacttgctcctggcctgtttgcacacgcaat

SEQ ID NO: 12 - DNA, 87 bp, Oldenlandia affinis
ggtcttccagtatgcggcgagacttgctttgggggaacttgcaacactccaggctgctcttgcacctggcctatctgcacacgcgat

SEQ ID NO: 13 - protein, 124 aa, Oldenlandia affinis
MAKFTVCLLLCLLLAAFVGAFGSELSDSHKTTLVNEIAEKMLQRKILDGVEATLVTDVAEKMFLRKMKAEAKTSETADQVFLKQLQLKGLPVCGETCVGGTCNTPGCTCSWPVCTRNGLPSLAA

SEQ ID NO: 14 - protein, 210 aa, Oldenlandia affinis
MAKFTNCLVLSLLLAAFVGAFGAEFSEADKATLVNDIAENIQKEILGEVKTSETVLTMFLKEMQLKGLPVCGETCFGGTCNTPGCSCTWPICTRDSLPMRAGGKTSETTLHMFLKEMQLKGLPVCGETCFGGTCNTPGCSCTWPICTRDSLPMSAGGKTSETTLHMFLKEMQLKGLPVCGETCFGGTCNTPGCSCTWPICTRDSLPLVAA

SEQ ID NO: 15 - DNA, 724 bp, Oldenlandia affinis
ggcaccagcactttcttaaaatttactgctttttcttatttcttgttctgtgcttgcttcttccatggctaagttcaccgtctgtctcctcctgtgcttgcttcttgcagcatttgttggggcgtttggatctgagctttctgactcccacaagaccaccttggtcaatgaaatcgctgagaagatgctacaaagaaagatattggatggagtggaagctactttggtcactgatgtcgccgagaagatgttcctaagaaagatgaaggctgaagcgaaaacttctgaaaccgccgatcaggtgttcctgaaacagttgcagctcaaaggacttccagtatgcggtgagacttgtgttgggggaacttgcaacactccaggctgcacttgctcctggcctgtttgcacacgcaatggccttcctagtttggccgcataatttgcttgatcaaactgcaaaaatgaatgagaaggccgacaccaataaagctatcaatgtagttggtccctgtacttaatttggttggctccaaaccatgtgtgctgctcttgtttttgttttttcttttttcttctctctttcgggcactcttcaggacatgaagtgatgatcagtactctttgctatcatgttttctgtgcacaccttctattgtaggtgttgttgtgatgttgatgcccaattggaataaactgttgtcgttgttaaaaaaaaaaaaaaaaa

SEQ ID NO: 16 - DNA, 993 bp, Oldenlandia affinis
ggcaccagatacaacccctttcttataatttattgcttttcttattccttgaaaaaggagaaataatattggatcttccatggctaagttcaccaactgtctcgtcctgagcttgcttctagcagcatttgttggggctttcggagctgagttttctgaagccgacaaggccaccttggtcaatgatatcgctgagaatatccaaaaagagatactgggcgaagtgaagacttctgaaaccgtccttacgatgttcctgaaagagatgcagctcaaaggtcttccagtatgcggcgagacttgctttgggggaacttgcaacactccaggctgctcttgcacctggcctatctgcacacgcgatagccttcctatgagggctggaggaaaaacatctgaaaccacccttcatatgttcctgaaagagatgcagctcaagggtcttccagtttgcggcgagacttgctttgggggaacttgcaacactccaggctgctcgtgcacctggcctatctgcacacgcgatagccttcctatgagtgctggaggaaaaacatctgaaaccacccttcatatgttcctgaaagagatgcagctcaagggtcttccagtttgcggcgagacttgctttgggggaacttgcaacactccaggctgctcgtgcacctggcctatatgcacacgtgatagccttcctcttgtggctgcataatttgcttcatcaaactgcaaaatgaataagaagggacactaaattagctatgaattttgttggcccttgtgtctggtaatttggttcccgccaaattaaccatatgtatgcattgctccttttttctttcttttttttccccctcatttgggcactcttcattacatgaagagatcatgacgctttgttactctgagcaccccctgttggtgttgttcacatgttgatgcccatgttggaataaactcttgtttttgttaccaaaaaaaaaaaaaaaaaaa

SEQ ID NO: 17 - protein, 29 aa, Artificial Sequence
  Mutant form of Kalata B1
  VARIANT 1: Xaa can be any amino acid
  VARIANT 8: Ala Cys Asp Glu Phe Gly His Ile Leu Met Asn Asn Pro Gln Arg Ser Thr Val Trp Tyr
  VARIANT 10: Cys Asp Glu Phe Gly His Ile Leu Met Asn Pro Gln Arg ?Ser Thr Val Trp Tyr
  VARIANT 18: Xaa can be any amino acid
  VARIANT 20: Xaa can be any amino acid
  VARIANT 22: Xaa can be any amino acid
  VARIANT 25: Xaa can be any amino acid
  VARIANT 29: Xaa can be any amino acid
XLPVCGEXCXGGTCNTPXCXCXWPXCTRX

SEQ ID NO: 18 - protein, 32 aa, Artificial Sequence
  Caripe-type cyclotide consensus sequence
  VARIANT 2: Xaa can be any amino acid
  VARIANT 6..8: Xaa can be any amino acid
  VARIANT 6..8: Xaa can be any amino acid
  VARIANT 10..13: Xaa can be any amino acid
  VARIANT 10..13: Xaa can be any amino acid
  VARIANT 15..17: Xaa can be any amino acid
  VARIANT 15..17: Xaa can be any amino acid
  VARIANT 19..21: Xaa can be any amino acid
  VARIANT 19..21: Xaa can be any amino acid
  VARIANT 23: Xaa can be any amino acid
  VARIANT 23: Xaa can be any amino acid
  VARIANT 25..28: Xaa can be any amino acid
  VARIANT 25..28: Xaa can be any amino acid
  VARIANT 31..32: Xaa can be any amino acid
  VARIANT 31..32: Xaa can be any amino acid
GXIPCXXXCXXXXCXXXAXXXCXCXXXXCYXX

SEQ ID NO: 19 - protein, 29 aa, Artificial Sequence
  Mutant form of Kalata B1
  VARIANT 2..4: Xaa can be any amino acid
  VARIANT 8: Xaa can be any amino acid
  VARIANT 10..13: Xaa can be any amino acid
  VARIANT 15..18: Xaa can be any amino acid
  VARIANT 20: Xaa can be any amino acid
  VARIANT 22..25: Xaa can be any amino acid
  VARIANT 27..29: Xaa can be any amino acid
GXXXCGEXCXXXXCXXXXCXCXXXXCXXX

SEQ ID NO: 20 - protein, 29 aa, Artificial Sequence
  [N29A]kalata B1
GLPVCGETCVGGTCNTPGCTCSWPVCTRA

SEQ ID NO: 21 - protein, 29 aa, Artificial Sequence
  [R28A]kalata B1
GLPVCGETCVGGTCNTPGCTCSWPVCTAN

SEQ ID NO: 22 - protein, 29 aa, Artificial Sequence
  [T27A]kalata B1
GLPVCGETCVGGTCNTPGCTCSWPVCARN

SEQ ID NO: 23 - protein, 29 aa, Artificial Sequence
  [V25A]kalata B1
GLPVCGETCVGGTCNTPGCTCSWPACTRN

SEQ ID NO: 24 - protein, 29 aa, Artificial Sequence
  [P24A]kalata B1
GLPVCGETCVGGTCNTPGCTCSWAVCTRN

SEQ ID NO: 25 - protein, 29 aa, Artificial Sequence
  [W23A]kalata B1
GLPVCGETCVGGTCNTPGCTCSAPVCTRN

SEQ ID NO: 26 - protein, 29 aa, Artificial Sequence
  [S22A]kalata B1
GLPVCGETCVGGTCNTPGCTCAWPVCTRN

SEQ ID NO: 27 - protein, 29 aa, Artificial Sequence
  [T20A]kalata B1
GLPVCGETCVGGTCNTPGCACSWPVCTRN

SEQ ID NO: 28 - protein, 29 aa, Artificial Sequence
  [G18A]kalata B1
GLPVCGETCVGGTCNTPACTCSWPVCTRN

SEQ ID NO: 29 - protein, 29 aa, Artificial Sequence
  [P17A]kalata B1
GLPVCGETCVGGTCNTAGCTCSWPVCTRN

SEQ ID NO: 30 - protein, 29 aa, Artificial Sequence
  [T16A]kalata B1
GLPVCGETCVGGTCNAPGCTCSWPVCTRN

SEQ ID NO: 31 - protein, 29 aa, Artificial Sequence
  [N15A]kalata B1
GLPVCGETCVGGTCATPGCTCSWPVCTRN

SEQ ID NO: 32 - protein, 29 aa, Artificial Sequence
  [T13A]kalata B1
GLPVCGETCVGGACNTPGCTCSWPVCTRN

SEQ ID NO: 33 - protein, 29 aa, Artificial Sequence
  [G12A]kalata B1
GLPVCGETCVGATCNTPGCTCSWPVCTRN

SEQ ID NO: 34 - protein, 29 aa, Artificial Sequence
  [G11A]kalata B1
GLPVCGETCVAGTCNTPGCTCSWPVCTRN

SEQ ID NO: 35 - protein, 29 aa, Artificial Sequence
  [T8A]kalata B1
GLPVCGEACVGGTCNTPGCTCSWPVCTRN

SEQ ID NO: 36 - protein, 29 aa, Artificial Sequence
  [E7A]kalata B1
GLPVCGATCVGGTCNTPGCTCSWPVCTRN

SEQ ID NO: 37 - protein, 29 aa, Artificial Sequence
  [G6A]kalata B1
GLPVCAETCVGGTCNTPGCTCSWPVCTRN

SEQ ID NO: 38 - protein, 29 aa, Artificial Sequence
  [V4A]kalata B1
GLPACGETCVGGTCNTPGCTCSWPVCTRN

SEQ ID NO: 39 - protein, 29 aa, Artificial Sequence
  [P3A]kalata B1
GLAVCGETCVGGTCNTPGCTCSWPVCTRN

SEQ ID NO: 40 - protein, 29 aa, Artificial Sequence
  [L2A]kalata B1
GAPVCGETCVGGTCNTPGCTCSWPVCTRN

SEQ ID NO: 41 - protein, 29 aa, Artificial Sequence
  [G1A]kalata B1
ALPVCGETCVGGTCNTPGCTCSWPVCTRN

SEQ ID NO: 42 - protein, 30 aa, Oldenlandia affinis
  kalata B19
GFPCGESCVYVPCLTAAIGCSCSNKVCYKN

SEQ ID NO: 43 - protein, 30 aa, Oldenlandia affinis
  kalata B18
GVPCAESCVYIPCISTVLGCSCSNQVCYRN

SEQ ID NO: 44 - protein, 30 aa, Oldenlandia affinis
  kalata B17
GIPCAESCVYIPCTITALLGCKCKDQVCYN

SEQ ID NO: 45 - protein, 30 aa, Oldenlandia affinis
  kalata B16
GIPCAESCVYIPCTITALLGCKCQDKVCYD

SEQ ID NO: 46 - protein, 29 aa, Oldenlandia affinis
  kalata B15
GLPVCGESCFGGSCYTPGCSCTWPICTRD

SEQ ID NO: 47 - protein, 30 aa, Oldenlandia affinis
  kalata B14
GLPVCGESCFGGTCNTPGCACDPWPVCTRD

SEQ ID NO: 48 - protein, 30 aa, Oldenlandia affinis
  kalata B13
GLPVCGETCFGGTCNTPGCACDPWPVCTRD

SEQ ID NO: 49 - protein, 28 aa, Oldenlandia affinis
  kalata B12
GSLCGDTCFVLGCNDSSCSCNYPICVKD

SEQ ID NO: 50 - protein, 29 aa, Oldenlandia affinis
  kalata B11
GLPVCGETCFGGTCNTPGCSCTDPICTRD

SEQ ID NO: 51 - protein, 30 aa, Oldenlandia affinis
  kalata B10 oia
GLPTCGETCFGGTCNTPGCSCSSWPICTRD

SEQ ID NO: 52 - protein, 30 aa, Oldenlandia affinis; identical to SEQ ID NO: 51
  kalata B10 linear

SEQ ID NO: 53 - protein, 30 aa, Oldenlandia affinis; identical to SEQ ID NO: 51
  kalata B10

SEQ ID NO: 54 - protein, 31 aa, Oldenlandia affinis
  kalata B9 linear
GSVFNCGETCVLGTCYTPGCTCNTYRVCTKD

SEQ ID NO: 55 - protein, 31 aa, Oldenlandia affinis; identical to SEQ ID NO: 54
  kalata B9

SEQ ID NO: 56 - protein, 29 aa, Oldenlandia affinis
  kalata B2 kyn
GLPVCGETCFGGTCNTPGCSCTWPICTRD

SEQ ID NO: 57 - protein, 29 aa, Oldenlandia affinis
  kalata B2 nfk
GLPVCGETCFGGTCNTPGCSCTWPICTRD

SEQ ID NO: 58 - protein, 29 aa, Oldenlandia affinis
  kalata B1 nfk
GLPVCGETCVGGTCNTPGCTCSWPVCTRN

SEQ ID NO: 59 - protein, 29 aa, Oldenlandia affinis
  kalata B1 oia
GLPVCGETCVGGTCNTPGCTCSWPVCTRN

SEQ ID NO: 60 - protein, 29 aa, Artificial Sequence
  [W19K, P20N, V21K]-kalata B1
GLPVCGETCVGGTCNTPGCTCSKNKCTRN

SEQ ID NO: 61 - protein, 29 aa, Artificial Sequence
  [P20D,V21K]-kalata B1
GLPVCGETCVGGTCNTPGCTCSWDKCTRN

SEQ ID NO: 62 - protein, 31 aa, Oldenlandia affinis
  kalata B8
GSVLNCGETCLLGTCYTTGCTCNKYRVCTKD

SEQ ID NO: 63 - protein, 30 aa, Oldenlandia affinis
  kalata B5
GTPCGESCVYIPCISGVIGCSCTDKVCYLN

SEQ ID NO: 64 - protein, 29 aa, Artificial Sequence
  kalata B1 IIa
GLPVCGETCVGGTCNTPGCTCSWPVCTRN

SEQ ID NO: 65 - protein, 29 aa, Oldenlandia affinis
  kalata S
GLPVCGETCVGGTCNTPGCSCSWPVCTRN

SEQ ID NO: 66 - protein, 29 aa, Oldenlandia affinis
  kalata B4
GLPVCGETCVGGTCNTPGCTCSWPVCTRD

SEQ ID NO: 67 - protein, 29 aa, Oldenlandia affinis
  kalata B7
GLPVCGETCTLGTCYTQGCTCSWPICKRN

SEQ ID NO: 68 - protein, 30 aa, Oldenlandia affinis
  kalata B3
GLPTCGETCFGGTCNTPGCTCDPWPICTRD

SEQ ID NO: 69 - protein, 30 aa, Oldenlandia affinis
  kalata B6
GLPTCGETCFGGTCNTPGCSCSSWPICTRN

SEQ ID NO: 70 - protein, 29 aa, Artificial Sequence
  kalata b1-6b
RNGLPVCGETCVGGTCNTPGCTCSWPVCT

SEQ ID NO: 71 - protein, 24 aa, Artificial Sequence
  kalata b1-6a
VCGETCVGGTCNTPGCTCSWPVCT

SEQ ID NO: 72 - protein, 27 aa, Artificial Sequence
  kalata b1-5
VCTRNGLPVCGETCVGGTCNTPGCTCS

SEQ ID NO: 73 - protein, 28 aa, Artificial Sequence
  kalata b1-4
CSWPVCTRNGLPVCGETCVGGTCNTPGC

SEQ ID NO: 74 - protein, 27 aa, Artificial Sequence
  kalata b1-3
GCTCSWPVCTRNGLPVCGETCVGGTCN

SEQ ID NO: 75 - protein, 29 aa, Artificial Sequence
  kalata b1-2
GTCNTPGCTCSWPVCTRNGLPVCGETCVG

SEQ ID NO: 76 - protein, 27 aa, Artificial Sequence
  kalata b1-1
TCVGGTCNTPGCTCSWPVCTRNLPVCG

SEQ ID NO: 77 - protein, 31 aa, Carapichea ipecacuanha
  caripe 1
GVIPCGESCVFIPCISTVIGCSCKDKVCYRN

SEQ ID NO: 78 - protein, 31 aa, Carapichea ipecacuanha
  caripe 2
GIPCGESCVFIRCTITALLGCSCSNNVCYKN

SEQ ID NO: 79 - protein, 30 aa, Carapichea ipecacuanha
  caripe 3
GIPCGESCVFIPCISAVVGCSCSNKVCYNN

SEQ ID NO: 80 - protein, 27 aa, Carapichea ipecacuanha
  caripe 4
LICSSTCLRIPCLSPRCTCRHHICYLN

SEQ ID NO: 81 - protein, 28 aa, Carapichea ipecacuanha
  caripe 5
  VARIANT 1: Xaa can be any amino acid
XCGESCVFIPCFTSVFGCSCKDKVCYRN

SEQ ID NO: 82 - protein, 28 aa, Carapichea ipecacuanha
  caripe 6
GAICTGTCFRNPCLSRRCTCRHYICYLN

SEQ ID NO: 83 - protein, 31 aa, Carapichea ipecacuanha
  caripe 7
GIPCGESCVFIPCTVTALLGCSCKNKVCYRN

SEQ ID NO: 84 - protein, 31 aa, Carapichea ipecacuanha
  caripe 8
GVIPCGESCVFIPCITAAIGCSCKKKVCYRN

SEQ ID NO: 85 - protein, 25 aa, Carapichea ipecacuanha
  caripe 9
  VARIANT 1: Xaa can be any amino acid
XCVFIPCTITALLGCSCSNNVCYKN

SEQ ID NO: 86 - protein, 31 aa, Carapichea ipecacuanha
  caripe 10
GVIPCGESCVFIPCFSTVIGCSCKNKVCYRN

SEQ ID NO: 87 - protein, 31 aa, Carapichea ipecacuanha
  caripe 11
GVIPCGESCVFIPCISTVIGCSCKKKVCYRN

SEQ ID NO: 88 - protein, 31 aa, Carapichea ipecacuanha
  caripe 12
GVIPCGESCVFIPCFSSVIGCSCKNKVCYRN

SEQ ID NO: 89 - protein, 30 aa, Carapichea ipecacuanha
  caripe 13
GIPCGESCVFIPCFTSVFGCSCKDKVCYRN

SEQ ID NO: 90 - protein, 29 aa, Viola tricolor
  viba 32
GLPVCGEACVGGTCNTPGCSCSWPVCTRN

SEQ ID NO: 91 - protein, 29 aa, Viola tricolor
  viba 30 linear
GPPVCGETCVGGTCNTPGCSCSWPVCTRN

SEQ ID NO: 92 - protein, 30 aa, Viola tricolor
  vitri peptide 18b
GSVFNCGETCVFGTCFTSGCSCVYRVCSKD

SEQ ID NO: 93 - protein, 31 aa, Viola tricolor
  vitri peptide 50
GDIPCGESCVYIPCITGVLGCSCSHNVCYYN

SEQ ID NO: 94 - protein, 33 aa, Viola tricolor
  vitri peptide 24a
GGTIFNCGESCFQGTCYTKGCACGDWKLCYGEN

SEQ ID NO: 95 - protein, 29 aa, Viola tricolor
  vitri peptide 39 linear
GAPICGESCFTGTCYTVQCSCSWPVCTRN

SEQ ID NO: 96 - protein, 29 aa, Viola tricolor
  vitri peptide 39
GAPICGESCFTGTCYTVQCSCSWPVCTRN

SEQ ID NO: 97 - protein, 28 aa, Viola tricolor
  vitri peptide 38
GDTCYETCFTGFCFIGGCKCDFPVCVKN

SEQ ID NO: 98 - protein, 33 aa, Viola tricolor
  vitri peptide 36/37
GGTIFSCGESCFQGTCYTKGCACGDWKLCYGEN

SEQ ID NO: 99 - protein, 28 aa, Viola tricolor
  vitri peptide 30
GFACGETCIFTSCFITGCTCNSSLCFRN

SEQ ID NO: 100 - protein, 32 aa, Viola tricolor
  vitri peptide 29
GVPSSDCLETCFGGKCNAHRCTCSQWPLCAKN

SEQ ID NO: 101 - protein, 31 aa, Viola tricolor
  vitri peptide 27a
GAFTPCGETCLTGECHTEGCSCVGQTFCVKK

SEQ ID NO: 102 - protein, 30 aa, Viola tricolor
  vitri peptide 24/28
GEPVCGDSCVFFGCDDEGCTCGPWSLCYRN

SEQ ID NO: 103 - protein, 29 aa, Viola tricolor
  vitri peptide 23
GLPTCGETCTLGTCYTPGCTCSWPLCTKN

SEQ ID NO: 104 - protein, 29 aa, Viola tricolor
  vitri peptide 94b
GVAVCGETCTLGTCYTPGCSCDWPICKRN

SEQ ID NO: 105 - protein, 29 aa, Viola tricolor
  vitri peptide 22a linear
GAPVCGETCFTGLCYSSGCSCIYPVCNRN

SEQ ID NO: 106 - protein, 29 aa, Viola tricolor
  vitri peptide 22a
GAPVCGETCFTGLCYSSGCSCIYPVCNRN

SEQ ID NO: 107 - protein, 31 aa, Viola tricolor
  vitri peptide 21
GGPLDCQETCTLSDRCYTKGCTCNWPICYKN

SEQ ID NO: 108 - protein, 32 aa, Viola tricolor
  vitri peptide 20
GDLVPCGESCVYIPCLTTVLGCSCSENVCYRN

SEQ ID NO: 109 - protein, 29 aa, Viola tricolor
  vitri peptide 18a
GVPICGETCFQGTCNTPGCTCKWPICERN

SEQ ID NO: 110 - protein, 33 aa, Viola tricolor
  vitri peptide 17
GSDDQVACGESCAMTPCFMHVVGCVCSQKVCYR

SEQ ID NO: 111 - protein, 28 aa, Viola tricolor
  vitri peptide 14
GSSCGETCEVFSCFITRCACIDGLCYRN

SEQ ID NO: 112 - protein, 32 aa, Viola tricolor
  vitri peptide 9a/53
GTIFDCGETCLLGKCYTPGCSCGSWALCYGQN

SEQ ID NO: 113 - protein, 29 aa, Viola tricolor
  vitri peptide 8
PTPCGETCIWISCVTAAIGCYCHESICYR

SEQ ID NO: 114 - protein, 30 aa, Viola tricolor
  vitri peptide 4
GTPCGESCIYVPCISAVFGCWCQSKVCYKD

SEQ ID NO: 115 - protein, 31 aa, Viola tricolor
  vitri peptide 3
GSWPCGESCVYIPCITSIAGCECSKNVCYKN

SEQ ID NO: 116 - protein, 31 aa, Viola tricolor
  vitri peptide 2
GSIPCGESCVWIPCISGIAGCSCSNKVCYLN

SEQ ID NO: 117 - protein, 31 aa, Viola tricolor
  vitri peptide 1
GLIPCGESCVWIPCISSVIGCSCKSKVCYKN

SEQ ID NO: 118 - protein, 29 aa, Viola odorata
  vocC
GLPVCGETCVGGTCNTPGCSCSWPVCIRN

SEQ ID NO: 119 - protein, 31 aa, Viola tricolor
  vigno 10
GTIPCGESCVWIPCISSVVGCSCKSKVCYKD

SEQ ID NO: 120 - protein, 30 aa, Viola tricolor
  vigno 9
GIPCGESCVWIPCISSALGCSCKSKVCYRN

SEQ ID NO: 121 - protein, 31 aa, Viola tricolor
  vigno 7
GTLPCGESCVWIPCISSVVGCSCKNKVCYKN

SEQ ID NO: 122 - protein, 31 aa, Viola tricolor
  vigno 6
GIPCGESCVWIPCISSAIGCSCKGSKVCYRN

SEQ ID NO: 123 - protein, 29 aa, Viola tricolor
  vigno 5
GLPLCGETCVGGTCNTPGCSCGWPVCVRN

SEQ ID NO: 124 - protein, 29 aa, Viola tricolor
  vigno 4
GLPLCGETCVGGTCNTPACSCSWPVCTRN

SEQ ID NO: 125 - protein, 29 aa, Viola tricolor
  vigno 3
GLPLCGETCVGGTCNTPGCSCSWPVCTRN

SEQ ID NO: 126 - protein, 31 aa, Viola tricolor
  vitri F
GTLPCGESCVWIPCISSVVGCACKSKVCYKD

SEQ ID NO: 127 - protein, 29 aa, Viola odorata
  vitri E
GLPVCGETCVGGTCNTPGCSCSWPVCFRN

SEQ ID NO: 128 - protein, 29 aa, Viola tricolor
  vitri D
GLPVCGETCFTGSCYTPGCSCNWPVCNRN

SEQ ID NO: 129 - protein, 29 aa, Viola tricolor
  vitri C
GLPICGETCVGGTCNTPGCFCTWPVCTRN

SEQ ID NO: 130 - protein, 29 aa, Viola tricolor
  vitri B
GYPICGESCVGGTCNTPGCSCSWPVCTTN

SEQ ID NO: 131 - protein, 29 aa, Viola abyssinica
  vaby C
GLPVCGETCAGGRCNTPGCSCSWPVCTRN

SEQ ID NO: 132 - protein, 30 aa, Viola odorata
  cycloviolacin O36
GLPTCGETCFGGTCNTPGCTCDPFPVCTHD

SEQ ID NO: 133 - protein, 31 aa, Viola odorata
  cycloviolacin O27
GSIPACGESCFKGWCYTPGCSCSKYPLCAKD

SEQ ID NO: 134 - protein, 31 aa, Viola odorata
  cycloviolacin O26
GSIPACGESCFRGKCYTPGCSCSKYPLCAKD

SEQ ID NO: 135 - protein, 30 aa, Viola odorata
  cycloviolacin O30
GIPCGESCVWIPCISSAIGCSCKNKVCFKN

SEQ ID NO: 136 - protein, 30 aa, Viola odorata
  cycloviolacin O29
GIPCGESCVWIPCISGAIGCSCKSKVCYKN

SEQ ID NO: 137 - protein, 29 aa, Viola odorata
  cycloviolacin O35
GLPVCGETCVGGTCNTPYCFCSWPVCTRD

SEQ ID NO: 138 - protein, 29 aa, Viola odorata
  cycloviolacin O34
GLPVCGETCVGGTCNTEYCTCSWPVCTRD

SEQ ID NO: 139 - protein, 29 aa, Viola odorata
  cycloviolacin O33
GLPVCGETCVGGTCNTPYCTCSWPVCTRD

SEQ ID NO: 140 - protein, 30 aa, Viola odorata
  cycloviolacin O32
GAPVCGETCFGGTCNTPGCTCDPWPVCTND

SEQ ID NO: 141 - protein, 29 aa, Viola odorata
  cycloviolacin O28
GLPVCGETCVGGTCNTPGCSCSWPVCFRD

SEQ ID NO: 142 - protein, 29 aa, Viola odorata
  cycloviolacin O31
GLPVCGETCVGGTCNTPGCSCSIPVCTRN

SEQ ID NO: 143 - protein, 30 aa, Viola baoshanensis
  Viba 11
GIPCGESCVWIPCISGAIGCSCKSKVCYRN

SEQ ID NO: 144 - protein, 30 aa, Viola baoshanensis
  Viba 9
GIPCGESCVWIPCISSAIGCSCKNKVCYRK

SEQ ID NO: 145 - protein, 30 aa, Melicytus ramiflorus
  Mra30
GIPCGESCVFIPCLTSAIGCSCKSKVCYRN

SEQ ID NO: 146 - protein, 29 aa, Viola baoshanensis
  Viba 15
GLPVCGETCVGGTCNTPGCACSWPVCTRN

SEQ ID NO: 147 - protein, 31 aa, Psychotria leptothyrsa
  vibi G
GTFPCGESCVFIPCLTSAIGCSCKSKVCYKN

SEQ ID NO: 148 - protein, 29 aa, Viola tricolor
  vibi C
GLPVCGETCAFGSCYTPGCSCSWPVCTRN

SEQ ID NO: 149 - protein, 31 aa, Viola odorata
  cycloviolacin O25
DIFCGETCAFIPCITHVPGTCSCKSKVCYFN

SEQ ID NO: 150 - protein, 30 aa, Viola odorata
  cycloviolacin O24
GLPTCGETCFGGTCNTPGCTCDPWPVCTHN

SEQ ID NO: 151 - protein, 31 aa, Viola odorata
  cycloviolacin O23
GLPTCGETCFGGTCNTPGCTCDSSWPICTHN

SEQ ID NO: 152 - protein, 29 aa, Viola odorata
  cycloviolacin O22
GLPICGETCVGGTCNTPGCTCSWPVCTRN

SEQ ID NO: 153 - protein, 29 aa, Viola odorata
  cycloviolacin O21
GLPVCGETCVTGSCYTPGCTCSWPVCTRN

SEQ ID NO: 154 - protein, 30 aa, Viola odorata
  cycloviolacin O20
GIPCGESCVWIPCLTSAIGCSCKSKVCYRD

SEQ ID NO: 155 - protein, 32 aa, Viola tricolor
  vitri peptide 100
GDPIPCGETCFTGKCYSETIGCTCEWPICTKN

SEQ ID NO: 156 - protein, 31 aa, Viola odorata
  cycloviolacin O19
GTLPCGESCVWIPCISSVVGCSCKSKVCYKD

SEQ ID NO: 157 - protein, 30 aa, Viola odorata
  cycloviolacin O18
GIPCGESCVYIPCTVTALAGCKCKSKVCYN

SEQ ID NO: 158 - protein, 30 aa, Viola odorata
  cycloviolacin O17
GIPCGESCVWIPCISAAIGCSCKNKVCYRN

SEQ ID NO: 159 - protein, 29 aa, Viola odorata
  cycloviolacin O16
GLPCGETCFTGKCYTPGCSCSYPICKKIN

SEQ ID NO: 160 - protein, 29 aa, Viola odorata
  cycloviolacin O15
GLVPCGETCFTGKCYTPGCSCSYPICKKN

SEQ ID NO: 161 - protein, 31 aa, Viola odorata
  cycloviolacin O14
GSIPACGESCFKGKCYTPGCSCSKYPLCAKN

SEQ ID NO: 162 - protein, 27 aa, Psychotria leptothyrsa
  violacin A
SAISCGETCFKFKCYTPRCSCSYPVCK

SEQ ID NO: 163 - protein, 30 aa, Viola odorata
  cycloviolacin O13
GIPCGESCVWIPCISAAIGCSCKSKVCYRN

SEQ ID NO: 164 - protein, 33 aa, Viola tricolor
  tricyclon B
GGTIFDCGESCFLGTCYTKGCSCGEWKLCYGEN

SEQ ID NO: 165 - protein, 33 aa, Viola tricolor
  tricyclon A
GGTIFDCGESCFLGTCYTKGCSCGEWKLCYGTN

SEQ ID NO: 166 - protein, 30 aa, Viola odorata
  cycloviolacin O1
GIPCAESCVYIPCTVTALLGCSCSNRVCYN

SEQ ID NO: 167 - protein, 30 aa, Viola tricolor
  varv peptide H
GLPVCGETCFGGTCNTPGCSCETWPVCSRN

SEQ ID NO: 168 - protein, 30 aa, Viola tricolor
  varv peptide G
GVPVCGETCFGGTCNTPGCSCDPWPVCSRN

SEQ ID NO: 169 - protein, 29 aa, Viola tricolor
  varv peptide F
GVPICGETCTLGTCYTAGCSCSWPVCTRN

SEQ ID NO: 170 - protein, 29 aa, Viola tricolor
  varv peptide D
GLPICGETCVGGSCNTPGCSCSWPVCTRN

SEQ ID NO: 171 - protein, 29 aa, Viola tricolor
  varv peptide C
GVPICGETCVGGTCNTPGCSCSWPVCTRN

SEQ ID NO: 172 - protein, 30 aa, Viola tricolor
  varv peptide B
GLPVCGETCFGGTCNTPGCSCDPWPMCSRN

SEQ ID NO: 173 - protein, 30 aa, Viola odorata
  cycloviolacin O10
GIPCGESCVYIPCLTSAVGCSCKSKVCYRN

SEQ ID NO: 174 - protein, 30 aa, Viola odorata
  cycloviolacin O7
SIPCGESCVWIPCTITALAGCKCKSKVCYN

SEQ ID NO: 175 - protein, 31 aa, Viola odorata
  cycloviolacin O6
GTLPCGESCVWIPCISAAVGCSCKSKVCYKN

SEQ ID NO: 176 - protein, 30 aa, Viola odorata
  cycloviolacin O5
GTPCGESCVWIPCISSAVGCSCKNKVCYKN

SEQ ID NO: 177 - protein, 30 aa, Viola odorata
  cycloviolacin O3
GIPCGESCVWIPCLTSAIGCSCKSKVCYRN

SEQ ID NO: 178 - protein, 30 aa, Viola tricolor
  cycloviolacin O4
GIPCGESCVWIPCISSAIGCSCKNKVCYRN

SEQ ID NO: 179 - protein, 29 aa, Viola odorata
  vodo N
GLPVCGETCTLGKCYTAGCSCSWPVCYRN

SEQ ID NO: 180 - protein, 29 aa, Viola thianshanica
  cycloviolacin O12
GLPICGETCVGGTCNTPGCSCSWPVCTRN

SEQ ID NO: 181 - protein, 29 aa, Viola baoshanensis
  kalata S
GLPVCGETCVGGTCNTPGCSCSWPVCTRN

SEQ ID NO: 182 - protein, 30 aa, Psychotria leptothyrsa
  vitri A
GIPCGESCVWIPCITSAIGCSCKSKVCYRN

SEQ ID NO: 183 - protein, 30 aa, Viola odorata
  cycloviolacin O9
GIPCGESCVWIPCLTSAVGCSCKSKVCYRN

SEQ ID NO: 184 - protein, 29 aa, Viola odorata
  vodo M
GAPICGESCFTGKCYTVQCSCSWPVCTRN

SEQ ID NO: 185 - protein, 31 aa, Viola odorata
  cycloviolacin O11
GTLPCGESCVWIPCISAVVGCSCKSKVCYKN

SEQ ID NO: 186 - protein, 31 aa, Viola odorata
  cycloviolacin O8
GTLPCGESCVWIPCISSVVGCSCKSKVCYKN

SEQ ID NO: 187 - protein, 30 aa, Viola philippica
  cycloviolacin O2
GIPCGESCVWIPCISSAIGCSCKSKVCYRN

(19) United States
(12) Patent Application Publication
(10) Pub. No.: US 2024/0269230 A1
(43) Pub. Date: Aug. 15, 2024

(54) CYCLOTIDES IN COMBINATION WITH KAPPA OPIOID RECEPTOR LIGANDS FOR MS THERAPY

(71) Applicant: MEDIZINISCHE UNIVERSITAT WIEN, Wien (AT)

(72) Inventors: Christian Gruber, Klosterneuburg (AT); Edin Muratspahic, Wien (AT)

(21) Appl. No.: 17/912,907

(22) PCT Filed: Mar. 19, 2021
(86) PCT No.: PCT/EP2021/057094

§ 371 Date: Sep. 20, 2022

(30) Foreign Application Priority Data
Mar. 20, 2020 (EP) 20164576.9

(51) Int. Cl. A61K 38/16; A61K 38/33; C07K 14/00

(52) U.S. Cl. CPC A61K 38/168; A61K 38/33; C07K 14/001

(57) ABSTRACT

The present invention relates to a pharmaceutical composition comprising a cyclotide and a ligand of the kappa opioid receptor (the kOR), or a combination thereof, for use in treating Multiple Sclerosis (MS), in remyelination, in improving CNS lesions, in preventing or reducing demyelination and/or CNS lesions, and/or in treating pain, in particular neuropathic pain and/or pain resulting from/coming along with MS. The present invention further relates to a combination of a cyclotide and a ligand of the kOR and to a pharmaceutical composition comprising said combination. The present invention further relates to a use of a cyclotide for reducing adverse effects of a ligand of the kOR and/or for increasing the potency and/or efficacy of a ligand of the kOR. Further, the present invention relates to a kit comprising a cyclotide and a ligand of the kOR. The present invention further relates to a pharmaceutical composition as part of a kit, wherein a comprised cyclotide and ligand of the kOR are for use in treating MS and related diseases and/or symptoms. The present invention further relates to a kit comprising a pharmaceutical composition comprising a cyclotide and a ligand of the kOR, wherein said pharmaceutical composition and/or said cyclotide and ligand of the kOR is/are for use in treating MS and related diseases and/or symptoms. The invention further relates to (a) novel Viola-type cyclotide(s).

The present invention relates to a pharmaceutical composition comprising a cyclotide and a ligand of the kappa opioid receptor (the kOR), or a combination thereof, for use in treating a demyelinating disease, neurological disorder and/or nerve-related disease, like Multiple Sclerosis (MS), in remyelination, in treating/improving CNS lesions, in preventing or reducing demyelination and/or CNS lesions, and/or in treating pain, in particular neuropathic pain, and/or pain resulting from/coming along or coupled with MS. The present invention further relates to a combination of a cyclotide and a ligand of the kOR and to a pharmaceutical composition comprising said combination. The present invention further relates to a use of a cyclotide for reducing adverse effects of a ligand of the kOR and/or for increasing the potency and/or efficacy of a ligand of the kOR. Further, the present invention relates to a kit comprising a cyclotide and a ligand of the kOR. The present invention further relates to a pharmaceutical composition as part of a kit, wherein a comprised cyclotide and ligand of the kOR are for use in treating MS and related diseases and/or symptoms. The present invention further relates to a kit comprising a pharmaceutical composition comprising a cyclotide and a ligand of the kOR, wherein said pharmaceutical composition and/or said cyclotide and ligand of the kOR is/are for use in treating a demyelinating disease, neurological disorder and/or nerve-related disease, like MS, and related diseases and/or symptoms. The invention further relates to (a) novel Viola-type cyclotide(s).

MS is a T-cell-mediated autoimmune disorder leading to inflammatory damage to the central nervous system (CNS) and causing disability in young adults. The pathogenesis of MS is characterized by a cascade of pathological events, involving the activation of the immune system, infiltration of lymphocytes, activation of microglia, focal inflammatory demyelination and axonal damage (Ciccarelli, Lancet Neurol 13 (8), 2014, 807-22). CD4⁺ T cells, particularly the Th17 and Th1 subgroups, have been suggested to initiate the disease (Sospedra, Annu Rev Immunol 23, 2005, 683-747).

All the currently available treatments for MS target the immune system with mechanisms of action including general immunosuppression/immunomodulation, such as beta-interferons, fingolimod and dimethyl fumarate, and blockade of immune cell infiltration into the CNS, such as natalizumab (Haghikia, Trends Mol Med 19 (5), 2013, 309-19). These drugs are effective in reducing the relapse rate and the formation of new lesions; however, they have very limited effects in preventing the progression of disability. Promoting oligodendrocyte progenitor cell (OPC) differentiation, remyelination and subsequent functional recovery of the neurons have been proposed to be the new direction of MS therapy (Plemel, Nat Rev Drug Discov 16 (9), 2017, 617-634; Franklin, Nat Rev Neurosci 18 (12), 2017, 753-769).

Further, in general, natural products have been and continue to be amongst the most valuable sources for drug discovery and development (Muratspahic, Trends Pharmacol Sci 40 (5), 2019, 309-326). The increasing interest for peptide-based drugs has intensified development of nature-derived peptides for therapeutic applications (Muratspahic, Trends Pharmacol Sci 40 (5), 2019, 309-326). Recently, the circular plant peptide kalata B1 was shown to silence T-cell proliferation in vitro in an IL-2-dependent mechanism (Grundemann, PLoS One 8 (6), 2013, e68016). Further, the in vivo activity of the mutant cyclotide [T20K]-kalata B1 using the MS EAE mouse model was shown (Thell, Proc Natl Acad Sci USA 113 (15), 2016, 3960-5; WO 2013/093045). Treatment of mice with this mutant cyclotide resulted in a significant delay and diminished symptoms of EAE by oral administration (Thell, loc. cit.). Inhibition of lymphocyte proliferation and the reduction of proinflammatory cytokines, in particular IL-2, distinguish the cyclotide from other marketed drugs (Thell, loc. cit.).

Further, the endogenous opioid system has been suggested to play a role in the pathogenesis of MS. In a Theiler's murine encephalomyelitis virus model of MS, mRNA levels of all the three opioid receptors, that is the mu, delta and kappa opioid receptors (mOR, dOR and kOR), were significantly decreased in the spinal cord (Lynch, Brain Res 1191, 2008, 180-91). In particular, the GPCR kOR, has recently been suggested to participate in the pathogenesis of MS (Du, Nat Commun 7, 2016, 11120; Mei, J Neurosci, 2016, 36 (30), 7925-35). The loss of opioid receptors could partially explain the central neuropathic pain commonly observed in the MS patients (Baron, Nat Clin Pract Neurol 2 (2), 2006, 95-106). Pregnant women have been reported to express higher levels of endogenous opioids (Akil, Annu Rev Neurosci 7, 1984, 223-55). Pregnant MS patients experience remission of the disease and have fewer relapses. However, three months after delivery, these women show a marked increase in relapse rate, in contrast to the decrease in endogenous opioid levels (Roullet, J Neurol Neurosurg Psychiatry 56 (10), 1993, 1062-5; Lutton, Exp Biol Med (Maywood) 229 (1), 2004, 12-20). Moreover, studies have recently demonstrated that genetic deletion of kOR induces a significantly severer phenotype of EAE (Du, loc. cit.; Mei, Nat Med 20 (8), 2014, 954-960; Mei, 2016, loc. cit.). The kOR does not affect T-cell differentiation and function, instead, it is critically involved in the differentiation of OPC towards myelinating oligodendrocytes (OLs) (Du loc. cit.). Hence, targeting the kOR by agonists such as U50,488 promotes OPC differentiation and remyelination, whereas the kOR knockout prevents agonist-mediated beneficial effects (Du, loc. cit.; Mei, 2014, loc. cit.; Mei, 2016, loc. cit.). Several studies reported an essential role of myelin for maintaining axonal integrity, presumably by providing physical and metabolic support to axons (Lee, Nature, 2012, 487 (7408), 443-8; Mei, 2014, loc. cit.). Recent efforts with high-throughput screening have identified a plethora of compounds that favours remyelination (Mei, 2014, loc. cit.; Najm, Nature, 2015, 522 (7555), 216-20). In WO 2019/171333, the use of the kOR agonist nalfurafine in the treatment of demyelination diseases is foreshadowed. Denny (Clinical & Translational Immunology 10(1), e1234, 2021, 1-19) discusses the promise of nalfurafine for clinical use in MS treatment in view of reduced neuroinflammation and promoted remyelination in models of CNS demyelinating disease. Further, Tangherlini (J. Med. Chem. 62, 2019, 893-907) describes the development of Quinoxaline-based kOR agonists for the treatment of neuroinflammtion. Moreover, [T20K]kalata B1 has been shown to bind to and activate hKOR (Muratspahic and Gruber, 2018, “Nature-derived peptides as molecular tools to study GPCR signaling” JOURNAL OF PEPTIDE SCIENCE 24, S137-S137, 2018).

However, the clinical potential of kOR agonists, such as U50,488, is limited due to deleterious side effects and off-target receptors. In particular, there are kOR agonists, such as U50,488, which, at the required pharmaceutically effective doses/concentrations, show adverse effects such as dysphoria, sedation, diuresis and/or hallucinations (Lalanne, Front Psychiatry, 2014, 5, 170). Recruitment of the cytosolic protein beta-arrestin 2 (β-arrestin 2) is known to be a relevant mechanism in this respect (Lalanne loc. cit.). Thus, continuous identification of novel compounds that are associated with reduced side effects and improved potency in the treatment of MS and related diseases/defects/symptoms is stringently required. Further, the variety of available MS drugs notwithstanding, there is still an unmet clinical need to develop new MS drugs with improved efficacy in preventing the progression of disability in MS patients.

Thus, the problem underlying the present invention is the provision of means and methods for an improved medical intervention in MS and related diseases, defects and/or symptoms, in particular for a medical intervention in MS and related diseases, defects and/or symptoms with no, less or ameliorated adverse effects.

The technical problem is solved by the provision of the embodiments characterized in the claims.

The present invention relates to a combination treatment of a demyelinating disease, neurological disorder and/or nerve-related disease, like MS, and related diseases, defects and/or symptoms, said combination treatment comprises the administration/medical use of a cyclotide and a ligand of the kOR. The treatment of neuroinflammation is also envisaged in this context.

The present invention relates to a combination treatment of MS and related diseases, defects and/or symptoms, said combination treatment comprises the administration/medical use of a cyclotide and a ligand of the kOR.

The present invention further relates to a pharmaceutical composition comprising
(a) a cyclotide; and
(b) a ligand of the kOR,
or a combination of (a) and (b),
for use in
(i) treating MS (e.g. treatment by therapy or prophylactic/preventive treatment);
(ii) remyelination (i.e. its induction or increase), in particular of oligodendrocytes, and/or improving (e.g. reducing or curing/healing) CNS lesions (e.g. brain lesions);
(iii) preventing or reducing demyelination (in particular of oligodendrocytes) and/or preventing the formation of (new) CNS lesions (e.g. brain lesions) and/or reducing existing CNS lesions (e.g. brain lesions); and/or
(iv) treating (e.g. treatment by therapy or prophylactic/preventive treatment) pain, in particular neuropathic pain (e.g. central or peripheral neuropathic pain) and/or pain resulting from/coming along with MS.

The present invention further relates to a pharmaceutical composition comprising
(a) a cyclotide; and
(b) a ligand of the kOR,
or a combination of (a) and (b),
for use in
(v) treating CNS lesions; and/or
(vi) treating a demyelinating disease, neurological disorder and/or nerve-related disease.

The present invention also relates to (a pharmaceutical composition comprising) a cyclotide for use, in combination with (a pharmaceutical composition comprising) a ligand of the kOR, in (i), (ii), (iii), (iv), (v) and/or (vi), supra.

The present invention also relates (a pharmaceutical composition comprising) a ligand of the kOR for use, in combination with (a pharmaceutical composition comprising) a cyclotide, in (i), (ii), (iii), (iv), (v) and/or (vi), supra.

The present invention further relates a method of
(i) treating MS (e.g. treatment by therapy or prophylactic/preventive treatment);
(ii) remyelination (i.e. its induction or increase), in particular of oligodendrocytes, and/or improving (e.g. reducing or curing/healing) CNS lesions (e.g. brain lesions);
(iii) preventing or reducing demyelination (in particular of oligodendrocytes) and/or preventing the formation of (new) CNS lesions (e.g. brain lesions) and/or reducing existing CNS lesions (e.g. brain lesions); and/or
(iv) treating (e.g. treatment by therapy or prophylactic/preventive treatment) pain, in particular neuropathic pain (e.g. central or peripheral neuropathic pain) and/or pain resulting from/coming along with MS,
said method comprising the step of administering to a subject in need thereof a pharmaceutically effective amount of
(a) a cyclotide; and
(b) a ligand of the kOR,
or a combination of (a) and (b), or a pharmaceutical composition comprising (a) and (b), or said combination.

The present invention further relates a method of
(v) treating CNS lesions; and/or
(vi) treating a demyelinating disease, neurological disorder and/or nerve-related disease.
said method comprising the step of administering to a subject in need thereof a pharmaceutically effective amount of
(a) a cyclotide; and
(b) a ligand of the kOR,
or a combination of (a) and (b), or a pharmaceutical composition comprising (a) and (b), or said combination.

The present invention solves the above identified technical problem since, as documented herein below and in the appended examples, it was surprisingly found that cyclotides are capable of binding to the kOR and, in particular, of acting as ligands of the kOR.

In a first experiment (see the appended Examples for details) peptide-enriched fractions from several plant species were shown to exhibit an affinity towards the kOR. Importantly, cyclotides isolated from Carapichea ipecacuanha and Viola tricolor were capable of binding to the kOR with an affinity in a low μM range. Notably, also the mutant cyclotide [T20K]-kalata B1 (also referred to herein as “T20K”, “[T20K]”, or “T20K per se/itself”; originally identified in Oldenlandia affinis; see, for example, WO 2013/093045) was shown herein as being able to bind and fully activate the kOR and to act as an orthosteric kOR ligand. Further, and importantly, [T20K]-kalata B1 was shown in the context of the present invention to act as an allosteric modulator of the kOR, thereby enhancing (as positive allosteric modulator, PAM) the efficacy/potency of defined (other) orthosteric kOR ligands. In particular, [T20K]-kalata B1 was shown in the context of the present invention is capable of affecting the potency/efficacy of kOR ligands such as dynorphin A 1-13 and U50,488. Moreover, it was surprisingly shown in the context of the present invention that [T20K]-kalata B1 itself does not recruit β-arrestin 2, and even reduces the efficacy of kOR ligands like U50,488 in recruiting β-arrestin 2. Thus, sound evidence is provided in the context of the present invention that cyclotides can be devoid of kOR-dependent and centrally-mediated adverse effects and even have the potential to decrease such effects of kOR agonists such as dynorphin A 1-13 and U50,488.

Thus, it was surprisingly demonstrated in the context of the invention that cyclotides, in particular [T20K]-kalata B1, can exhibit a bitopic mode of action at the kOR, in that they act as orthosteric ligands as well as allosteric modulators, and that their action(s) come along with decreased kOR-dependent and centrally-mediated adverse effects. In other words, it was surprisingly demonstrated in the context of the invention that cyclotides, in particular [T20K]-kalata B1, can not only be orthosteric ligands at the kOR, but can also act as bitopic ligands capable of engaging the kOR in an allosteric manner.

Finally, the ability of [T20K]-kalata B1 to penetrate the CNS in the EAE model was demonstrated herein and in the appended examples.

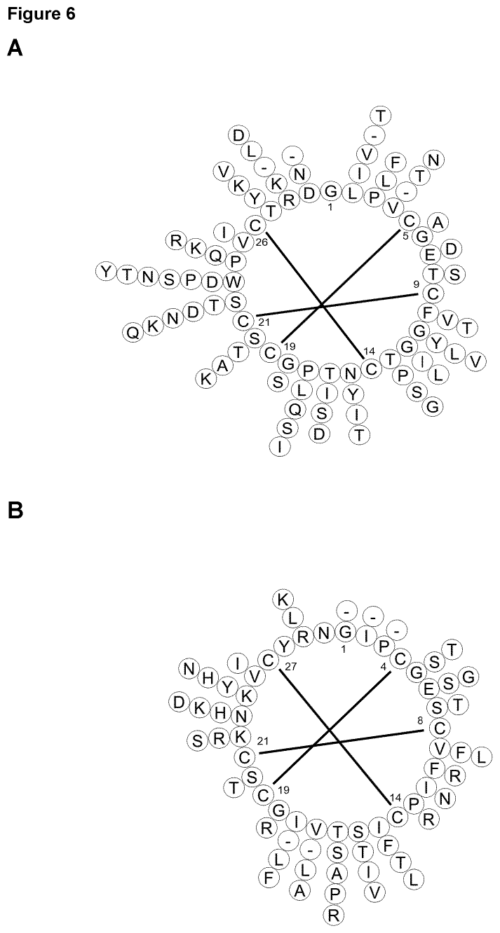

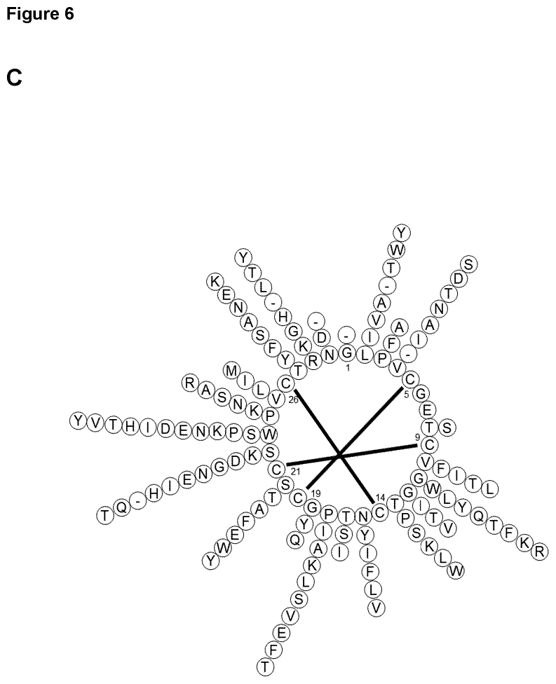

Thus, there is evidence provided in the context of the present invention that targeting the kOR by a combination of (a) cyclotide(s), in particular [T20K]-kalata B1, and (a) kOR ligand(s), in particular (an) orthosteric kOR agonist(s), like dynorphin A 1-13 or U50,488, has a high potential in therapies of demyelinating diseases, neurological disorders and/or nerve-related diseases, like remyelination and/or pain therapies and, more particular, MS (remyelination and/or pain) therapies, which, in addition, promise beneficial immunosuppressant effects. Furthermore, their sequence diversity renders cyclotides as being ideal candidates for therapies of demyelinating diseases, neurological disorders and/or nerve-related diseases, like remyelination and/or pain therapies and, more particular, MS (remyelination and/or pain) therapies, when combined with kOR ligands (cf. also appended FIGS. 6A, B and C).

The treatment, in particular the MS treatment and the treatment of related diseases, defects and/or symptoms, to be applied in the context of the invention may comprise or result in a decrease of the relapse rate and/or of the frequency of MS episodes and/or the prevention or decrease of progression of disability (of the patient pertained).

Further, the treatment, in particular the treatment of MS and of related diseases, defects and/or symptoms, to be applied in the context of the invention may comprise or result in
(ii) remyelination (i.e. its induction or increase), in particular of oligodendrocytes, and/or improving (e.g. reducing or curing/healing) CNS lesions (e.g. brain lesions);
(iii) preventing or reducing demyelination (in particular of oligodendrocytes) and/or preventing the formation of CNS lesions (e.g. brain lesions) and/or reducing existing CNS lesions (e.g. brain lesions); and/or
(iv) treating (e.g. treatment by therapy or prophylactic/preventive treatment) pain, in particular neuropathic pain (e.g. central or peripheral neuropathic pain) and/or pain resulting from/coming along with MS.

Further, in the context of the treatment, in particular the treatment of MS and of related diseases, defects and/or symptoms, of the invention,
(i) remyelination, in particular of oligodendrocytes, is to be induced or increased and/or CNS lesions (e.g. brain lesions) are to be improved (e.g. reduced or cured/healed);
(ii) demyelination, in particular of oligodendrocytes, is to be prevented or reduced and/or the formation of CNS lesions (e.g. brain lesions) is to be prevented and/or existing CNS lesions (e.g. brain lesions) are to be reduced; and/or
(iii) pain, in particular neuropathic pain (e.g. central or peripheral neuropathic pain) and/or pain resulting from/coming along with MS is to be treated.

In the context of the invention, the treatment, in particular of MS-related diseases, defects and/or symptoms, for example, are envisaged to mean (the formation of) (new) CNS lesions (e.g. brain lesions), demyelinisation (of oligodendrocytes), (central or peripheral) neuropathic pain, pain resulting from/coming along with MS, (the progression of) disability and the like.

The treatment, in particular the treatment of MS and of related diseases, defects and/or symptoms, may also comprise or result in promoting OPC differentiation, remyelination and/or (subsequent) functional recovery of neurons.

The meanings of demyelinating diseases, neurological disorders and/or nerve-related diseases, in particular of MS and related diseases, defects and symptoms, are well known by the skilled person. They are, for example, described in Ciccarelli (loc. cit.) and Sospedra (loc. cit.).

Examples of demyelinating diseases, neurological disorders and/or nerve-related diseases to be treated in accordance with the inventions are selected from the group consisting of MS, optic neuritis, Devic's disease, inflammatory demyelinating diseases, central nervous system neuropathies, myelopathies (like Tabes dorsalis), leukoencephalopathies and leukodystrophies or is selected from the group consisting of Guillain-Barre syndrome and its chronic counterpart, chronic inflammatory demyelinating polyneuropathy, anti-MAG (myelin-associated glycoprotein) peripheral neuropathy, Charcot Marie Tooth (CMT) disease, copper deficiency and progressive inflammatory neuropathy.

In the context of one aspect of the invention, adverse effects, in particular kOR-dependent adverse effects (centrally-mediated or peripherally-mediated), more particular adverse effects resulting from β-arrestin 2 recruitment, are to be reduced/ameliorated or avoided. Such adverse effects are, for example, (adverse effects related to) opioid crisis/tolerance and/or dysphoria, sedation, diuresis and/or hallucinations (cf. Lalanne loc cit). Thus, in the context of one aspect of the treatment of MS and related diseases, defects and/or symptoms according to the invention, one or more of such adverse effects is/are to be reduced/ameliorated or avoided.

It is preferred that the kOR to be targeted in the context of the invention is the human kOR (hkOR). A preferred kOR ligand is thus a hkOR ligand. In general, it is preferred that the kOR to be targeted is the kOR of the patient to be treated. For example, if the patient to be treated and is human, the kOR to be targeted preferably is the hkOR.

The kOR, in particular the hkOR, is well known in the art and is, for example, described in Lalanne, loc. cit. and Du, loc. cit.). A detailed characterization of the hkOR, in particular the amino acid sequence information, is derivable from the database entry https://www.uniprot.org/uniprot/P41145.

The meaning of “(h)kOR ligand/agonist” and “ligand/agonist of the (h)kOR” is known in the art and the respective terms are used herein accordingly (e.g. https://www.guidetopharmacology.org/GRAC/ObjectDislayForward?objectId=318&fam ilyId=50&familyType=GPCR).

In the context of the invention, the ligand of the kOR may be an agonist of the kOR (“unbiased” ligand/“unbiased” agonist of the kOR”), a partial agonist of the kOR or a biased agonist of the kOR.

In the context of the invention, being an “agonist” of the kOR and exhibiting “agonistic” function on the kOR, respectively, means that the kOR is activated, i.e. its relevant biological function(s) is(are) induced or increased. In particular, being an “agonist” of the kOR and exhibiting “agonistic” function on the kOR, respectively, means in the context of the invention that the response of kOR to an opioid stimulus, e.g. the intracellular cAMP reduction, is induced or increased, leading to activation of the RAF/MEK_{1/2}/ERK_{1/2} or the JAK2/STAT3 signalling cascades (https://pubmed.ncbi.nlm.nih.gov/27881770-enhancing-remyelination-through-a-novel-opioid-receptor-pathway/?from_single_result=borniger+and+hesp; Borniger, J Neurosci 36(47), 2016, 3). Assays for testing the relevant biological function of the kOR are known in the art and are, for example, described in https://pubmed.ncbi.nlm.nih.gov/27881770-pathway/?from_single_result=borniger+and+hesp. Further, such an assay is described in the appended examples (e.g. Examples 2 and 3). Multiple agonists of the kOR are known in the art https://www.guidetopharmacology.org/GA/ObjectDisplayForward?objectId=318&fam ilyId=&familyType=GC. Several non-limiting examples of kOR agonists are given in appended Table 6.

Usually, agonists of the kOR are full ligands/agonists, i.e. “unbiased” ligands/agonists. This means that they fully activate a kOR signaling pathway, e.g. either specific pathway, i.e. the G protein pathway, and/or the arrestin pathway. For example, in the context of the invention, an “unbiased” ligand/agonist of the kOR is a ligand/agonist which induces or is capable of inducing arrestin recruitment, in particular β-arrestin 2 recruitment, or is a ligand/agonist which increases or is capable of increasing arrestin recruitment, in particular β-arrestin 2 recruitment; in particular at the relevant endogenous or pharmaceutically effective concentrations/doses. Without being bound by theory, arrestin recruitment, in particular β-arrestin 2 recruitment, may be associated with higher (and/or faster) internalization of the kOR. This, in turn, may enhance the chance for, for example, tolerance (cf. opioid crisis). Since arrestin recruitment, in particular β-arrestin 2 recruitment, is associated with adverse effects, such as opioid crisis/tolerance and/or dysphoria, sedation, diuresis and/or hallucinations (Lalanne loc cit), fully agonistic, “unbiased” kOR ligands usually show adverse effects, such as opioid crisis/tolerance and/or dysphoria, sedation, diuresis and/or hallucinations (Lalanne loc cit), in particular at the required pharmaceutically effective concentrations/doses.

Multiple fully agonistic, “unbiased” kOR ligands are known in the art. Several non-limiting examples of such kOR agonists are given in appended Table 6 and simply termed as “Agonist”.

Besides fully agonistic, “unbiased” kOR ligands/agonists, ligands/agonists of the kOR may also be “biased” ligands/agonists. This means that they do not (or to a lower degree) activate a kOR signaling pathway, e.g. either specific pathway, i.e. the G protein pathway, or the arrestin pathway. For example, in the context of the invention, a “biased” ligand/agonist of the kOR is a ligand/agonist which does not (or to a lower degree) induce, or which is not (or to a lower degree) capable of inducing, arrestin recruitment, in particular β-arrestin 2 recruitment, or is a ligand/agonist which does not (or to a lower degree) increase, or which is not (or to a lower degree) capable of increasing, arrestin recruitment, in particular β-arrestin 2 recruitment, in particular at the relevant endogenous or pharmaceutically effective concentrations/doses. In the context of the invention, “biased” kOR ligands/agonists show less or no adverse effects, such as opioid crisis/tolerance and/or dysphoria, sedation, diuresis and/or hallucinations, in particular at the required pharmaceutically effective concentrations/doses. For example, in the context of the invention, G-protein biased ligands/agonists show reduced side effects. Multiple “biased” kOR ligands/agonists are known in the art. Several non-limiting examples of such kOR agonists are given in appended Table 6 and are termed “Biased agonist”.

It will be appreciated by the person skilled in the art that the “bias” of kOR ligands/agonists and their “bias factor”, respectively, is not an absolute characteristic and may vary within some ranges. In particular, there is a “smooth transition” from “unbiased” kOR ligands/agonists to “biased” kOR ligands/agonists.

However, there are typical “biased” kOR ligands/agonists with respect to a kOR signaling pathway. Examples of typical “biased” kOR ligands/agonists are known in the art and are given in appended Table 6 (“Biased agonist”). It is preferred that “biased” kOR ligands/agonists are naturally occurring “biased” kOR ligands/agonists. Particular non-limiting examples of typical “biased” kOR ligands/agonists to be used in accordance with the invention are collybolide (mushroom Collybia maculate), noribogaine (metabolite of plant iboga), B-64 (Salvinorin A derivative), nalfurafine (morphine derivative), triazole1.1, 6-GNTI, HS666, HS665 and mesyl salvinorin B.

Likewise, there are typical “unbiased” kOR ligands/agonists with respect to a kOR signaling pathway. Examples of typical “unbiased” kOR ligands/agonists are known in the art and are given in appended Table 6 (“Agonist”). Particular non-limiting examples of typical “unbiased” kOR ligands/agonists to be used in accordance with the invention are dynorphin A-(1-13), dynorphin-(1-11), dynorphin A, dynorphin A-(1-8), U50488, U69593, GR 89696, spiradoline, BRL-52537, JT09, difelikefalin (CR845, FE-202845), also known as D-Phe-D-Phe-D-Leu-D-Lys-[γ-(4-N-piperidinyl)amino carboxylic acid), dynorphin, nalbuphine, pentasozin, pethidine and sulfentanil.

The skilled person knows, or is at least readily able to test, the “bias factor” of a given kOR ligand/agonist. Respective assays are described in the appended Examples and are known in the art (see, for example, https://www.ncbi.nlm.nih.gov/pmc/articles/PMC3868907 (White, Mol Pharmacol 85(1), 2014, 83-90) and https://www.ncbi.nlm.nih.gov/pubmed/25320048 (White, J Pharmacol Exp Ther 352(1), 2015, 98-109)).

Besides fully agonistic, “unbiased” kOR ligands/agonists, ligands/agonists of the kOR may also be “partial” ligands/agonists. Being a “partial” kOR ligand/agonist means that the ligand/agonist has have less efficacy (for a given pathway, e.g. the G-protein or the arrestin or other pathway; typically for the G protein pathway) as compared to the endogenous comparison/standard ligand. For a non-limiting illustration, Emax is reduced between 1-99%; 0% is an antagonist and 100% being the endogenous full agonist. In practical terms, a “partial” kOR ligand/agonist may have about 20-80% of the efficacy of the endogenous full ligand/agonist.

Multiple “partial” kOR ligands/agonists are known in the art. Several non-limiting examples of such kOR agonists are given in appended Table 6 and are termed “Partial agonist”. Particular non-limiting examples of typical “partial” kOR ligands/agonists are dynorphin B, DAMGO, and endomorphin-1-Amo2.

In general, the ligand of the kOR to be used in accordance with the invention may be a small molecule or a peptide ligand of the kOR, for example an endogenous peptide ligand of the kOR. Respective ligands are known in the art. Respective examples are given in appended Table 6 and termed “Small molecule”, “Peptide” or “endogenous”, respectively. Particular, however non-limiting, examples of small molecules which are ligands, in particular agonists, of the kOR are also described in Tangherlini (loc. cit.), Bourgeois (J. Med. Chem. 57, 2014, 6845-60), Molenveld (Bioorg. Med. Chem. Lett. 25, 2015, 5326-30) and Soeberdt (J. Med. Chem. 60, 2017, 2526-51). In one aspect, Quinoxaline-based kOR agonists may be used according to the invention (like those described in Tangherlini (loc. cit.), Bourgeois ((loc. cit.), Molenveld (loc. cit.). Examples of such Quinoxaline-based kOR agonists are compound 12 and compound 14 (as, for example, described in Tangherlini (loc. cit.); see sections 4.2.1. and 4.2.3. thereof, and also Table 6, infra).

Particular non-limiting examples of a ligand of the kOR to be used in the context of the invention is U50,488 or dynorphin A-(1-13).

It is preferred that the (h)kOR ligand/agonist to be used is an orthosteric (h)kOR ligand/agonist. This means that it binds to the same site of the (h)kOR as an endogenous ligand or is an endogenous ligand.

It is envisaged in the context of the invention that the (h)kOR ligand/agonist to be used (item (b)) is not the cyclotide to be used (item (a)). Thus, the (h)kOR ligand/agonist to be used is envisaged to be a different, additional active ingredient (in the pharmaceutical composition, the kit or the combination etc. of the invention). It is preferred that that the (h)kOR ligand/agonist to be used is not a cyclotide at all.

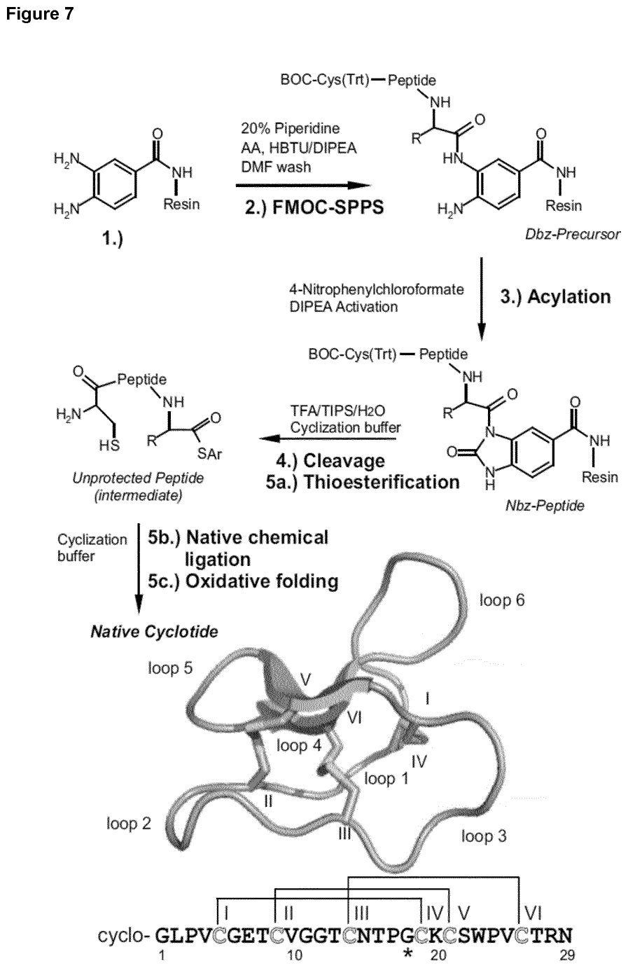

In general, the meaning of the term “cyclotide” is known in the art and the term “cyclotide” is used herein accordingly (see, for example, CyBase at http://www.cybase.org.au/index.php). In particular, “cyclotides” as used in the context of the invention are head-to-tail cyclized peptides which cyclotide chain includes six conserved cysteine residues capable to form three disulfide bonds arranged in a cyclic cystine-knot (CCK) motive (cyclotide chain; cf. FIG. 7). The inter-cysteine sequences of a cyclotide can tolerate a wide range of residue substitutions (see, for example, Clark, 2006, Biochem J, 394, 85-93 and FIGS. 6 and 7). In one aspect, the term “cyclotide” used herein refers to cyclotides as described in Craik (1999, J Mol Biol, 294, 1327-1336), Clark (2006, loc. cit.) and, in particular, in U.S. Pat. No. 7,592,533 B1.

A “cyclotide” to be employed in the context of the invention may include the typical Glu (E) residue in loop 1 (cf. FIG. 7). However, also other, “cyclotides” may be employed. For example some cyclotides of the caripe type do not contain the typical E in loop 1 (cf. Fahradpour Front Pharmacol 8, 2017, 616). They are, nevertheless, envisaged to be encompassed by the term “cyclotide” in accordance with the invention. The meaning of the term “cyclotide” also encompasses “cyclic knottins”, like the mentioned caripe-type cyclic knottins which do not contain the typical E in loop 1.

In particular, a cyclotide to be used in the context of the invention comprises an amino acid sequence capable of forming a cyclic backbone, wherein said cyclic backbone comprises the structure (formula I):

Cyclo(C[X₁. . . Xₐ]C[X^{I}₁. . . X^{I}_{b}]C[X^{II}₁. . . X^{II}_{c}9 C[X^{III}₁. . . X^{III}₁. . . X^{III}_{d}]C[X^{IV}₁. . . X^{IV}ₑ]C[X^{V}₁. . . X^{V}_{f}])
wherein
(i) C is cysteine;
(ii) each of [X₁ . . . Xₐ], [X^{I}₁ . . . X^{I}_{b}], [X^{II}₁ . . . X^{II}_{c}], [X^{III}₁′ . . . X^{III}_{d}], [X^{IV}₁ . . . X^{IV}ₑ], and [X^{V}₁ . . . X^{V}_{f}] represents one or more amino acid residues, wherein each one or more amino acid residues within or between the sequence residues may be the same or different; and
(iii) a, b, c, d, e, and f represent the number of amino acid residues in each respective sequence and each of a to f may be the same or different and range from 1 to about 20.

Preferably, a is 3 to 6, b is 4 to 8, c is 3 to 10, d is 1, e is 4 to 8, and/or f is 5 to 13.

Particular cyclotides which may be used/administered in the context of the invention are kalata type cyclotides (for example kalata B-type cyclotides, like kalata B1 or a mutant thereof or kalata B2 or a mutant thereof; see also Table 3 and FIG. 6A), Caripe-type cyclotides (for example any of Caripe1 to Caripe13 or a mutant thereof or Psyle E or a mutant thereof; see also Table 4 and FIG. 6B) or Viola-type cyclotides (for example any of the Viola-type cyclotides as listed in Table 5 or a mutant thereof, in particular the novel “vitri” cyclotide (also termed “vitri peptide 100”) that has been identified in the context of the present invention (GDPIPCGETCFTGKCYSETIGCTCEWPICTKN) or a mutant thereof; see also Table 5 and FIG. 6C).

Further, particular cyclotides to be used/administered in accordance with the invention may be derived from, or may be comprised in, an extract of Oldenlandia affinis, Viola tricolor, Carapichea ipecacuanha, Psychotria solitudinum, Viola odorata, Momordica charantia or Beta vulgaris. An extract of Oldenlandia affinis, Viola tricolor, and Carapichea ipecacuanha is preferred).

Non-limiting examples of kalata B-type cyclotides are depicted in Table 3 and FIG. 6A.

Non-limiting examples of Caripe-type cyclotides are depicted in Table 4 and FIG. 6B.

Non-limiting examples of Viola-type cyclotides are depicted in Table 5 and FIG. 6C.

Kalata B-type cyclotides are known in the art and are, for example, described in WO2013/093045, Grundemann (JNatProt 75(2), 2012, 167-74; 2013 loc. cit.), Hellinger (J Ethnopharmacol 151(1), 2014, 299-306) and Thell (loc. cit.). Caripe-type cyclotides are known in the art and are, for example, described in Fahradpour (loc. cit.). Viola-type cyclotides are also known in the art and are, for example, described in Hellinger (J Proteome Res 14(11), 2015, 4851-62).

A cyclotide to be used/administered in the context of the invention, in particular a kalata B-type cyclotide, may comprise the amino acid stretch of formula II (SEQ ID NO. 17)

Xxx₁-Leu-Pro-Val-Cys-Gly-Glu-Xxx₂-Cys-Xxx₃-Gly-
Gly-Thr-Cys-Asn-Thr-Pro-Xxx₁-Cys-Xxx₁-Cys-Xxx₁-
Trp-Pro-Xxx₁-Cys-Thr-Arg-Xxx₁ (II).

Xxx₁, Xxx₂ and Xxx₃ may be any amino acid, non-natural amino acid or peptidomimetic, preferably an aliphatic amino acid. In particular, Xxx₂ may be any amino acid, non-natural amino acid or peptidomimetic but not Lys and/or Xxx₃ may be any amino acid, non-natural amino acid or peptidomimetic but not Ala or Lys. Preferably, Xxx₂ and/or Xxx₃ of formula II are not mutated at all. More particular, Xxx₁ may be Gly, Thr, Ser, Val, IIe, Asn, Asp or, preferably, Lys, Xxx₂ may be Thr, and/or Xxx₃ may be Val or Phe. Even more particular, Xxx₁ at position 1 of formula II may be Gly, Xxx₁ at position 18 of formula II may be Lys or, preferably, Gly, Xxx₁ at position 20 of formula II may be Thr, Ser or, preferably, Lys, Xxx₁ at position 22 of formula II may be Ser or Thr, Xxx₁ at position 25 of formula II may be Val or IIe, Xxx₁ at position 29 of formula II may be Asn, Asp or, preferably, Lys, Xxx₂ of formula II may be Thr and/or Xxx₃ of formula II may be Val or Phe.

The specifically defined amino acid residues of formula II may also vary depending on the particular (type of) cyclotide. Hence, what has been said with respect to Xxx₁, Xxx₂ and/or Xxx₃, does not only apply to formula II but also to the corresponding amino acid residues of other cyclotides not comprising the particular amino acid stretch of formula II. In this context, “corresponding” particularly means amino acid residues at the same or similar position(s). More particular, “corresponding” means being homologous. Examples of other cyclotides are the herein defined Caripe-type cyclotides, in particular the below defined cyclotides comprising the amino acid stretch of formula III (SEQ ID NO. 18); and the herein defined Viola-type cyclotides, in particular the below defined cyclotides comprising the amino acid stretch of formula IIII (SEQ ID NO. 19).

A cyclotide to be used/administered in the context of the invention, in particular a Caripe-type cyclotide, may comprise the amino acid stretch of formula III (SEQ ID NO. 18)

(SEQ ID NO. 18)
Gly-Xxx₁-Ile-Pro-Cys-Xxx₂-Xxx₃-Xxx₄-Cys-Xxx₅-
Xxx₆-Xxx₇-Xxx₈-Cys-Xxx₉-Xxx₁₀-Xxx₁₁-Ala-Xxx₁₂-
Xxx₁₃-Xxx₁₄-Cys-Xxx₁₅-Cys-Xxx_{16 or}-Xxx₁₇-Xxx₁₈-
Xxx₁₉-Cys-Tyr-Xxx₂₀-Xxx₂₁

Any or all of Xxx₁ to Xxx₂₁ may be any amino acid, non-natural amino acid or peptidomimetic. In particular, Xxx₁ may be Val, Ala or Leu, Xxx₂ may be Gly, Ser or Thr, Xxx₃ may be Glu, Gly or Ser, Xxx₄ may be Ser or Thr, Xxx₅ may be Val, Leu or Phe, Xxx₆ may be Phe or Arg, Xxx₇ may be IIe or Asn, Xxx₈ may be Pro or Arg, Xxx₉ may be IIe, Phe, Thr or Leu, Xxx₁₀ may be Ser, Thr, IIe or Val, Xxx₁₁ may be Thr, Ser, Ala, Arg or Pro, Xxx₁₂ may be Val, Leu or Ala, Xxx₁₃ may be IIe, Leu, Phe or Val, Xxx₁₄ may be Gly or Arg, Xxx₁₅ may be Ser or Thr, Xxx₁₆ may be Lys, Ser or Arg, Xxx₁₇ may be Asn, Asp, His or Lys, Xxx₁₈ may be Lys, Asn, His or Tyr, Xxx₁₉ may be Val or IIe, Xxx₂₀ may be Arg, Leu, Lys or Asn and/or Xxx₂₁ may be Asn or Asp.

A cyclotide to be used/administered in the context of the invention, in particular a Viola-type cyclotide, may comprise the amino acid stretch of formula IIII (SEQ ID NO. 19)

(SEQ ID NO. 19)
Gly-Xxx₁-Xxx₂-Xxx₃-Cys-Gly-Glu-Xxx₄-Cys-Xxx₅-
Xxx₆-Xxx₇-Xxx₈-Cys-Xxx₉-Xxx₁₀-Xxx₁₁-Xxx₁₂-Cys-
Xxx₁₃-Cys-Xxx₁₄-Xxx₁₅-Xxx₁₆-Xxx₁₇-Cys-Xxx₁₈-
Xxx₁₉-Xxx₂₀

Any or all of Xxx₁ to Xxx₂₀ may be any amino acid, non-natural amino acid or peptidomimetic. Preferably, any or all of Xxx₁ to Xxx₂₀ may be (a) conservative amino acid exchange(s) of the corresponding amino acid residue(s) of the “vitri” cyclotide as depicted in SEQ ID NO. 155.

A cyclotide to be used/administered in the context of the invention may be a mutated form of a native cyclotide.

In general, “mutation” in the context of the present invention means any change in the structure of the (native or wildtype) cyclotide, in particular in the primary amino acid sequence thereof. More particular, “mutation” means that one or more amino acid residues of the (native or wildtype) cyclotide are replaced, substituted or added. In one specific aspect, “mutation” refers to a point mutation, i.e. to the replacement, substitution or addition of one amino acid residue. In a more specific aspect, “mutation” refers to the replacement of one amino acid residue. What has been said with respect to the mutated/variant forms of cyclotides to be used in accordance with the present invention and with respect to the respective mutations herein elsewhere also applies to the meaning of the term “mutation”, mutatis mutandis.

A mutated form of a (native) cyclotide in accordance with the intention may have at least one of the amino acid residues corresponding to (e.g. being homologous to) Xxx₁ and Xxx₄₋₈ of formula II, preferably corresponding to (e.g. being homologous to) Xxx₅ at position 20 of formula II, replaced by (a) different amino acid residue(s). Non-limiting examples of such mutated forms are cyclotides which have one (preferred), two or more of the amino acid residues corresponding to (e.g. being homologous to) amino acid position 1, 18, 20 and/or 29 of SEQ ID NO: 1 or 2, preferably corresponding to (e.g. being homologous to) amino acid position 18, 20 and/or 29 of SEQ ID NO: 1 or 2, more preferably corresponding to (e.g. being homologous to) amino acid position 20 and/or 29 of SEQ ID NO: 1 or 2, most preferably corresponding to (e.g. being homologous to) amino acid position 20 of SEQ ID NO: 1 or 2, replaced by (a) different amino acid residue(s).

A particular mutation in the mutated form of a cyclotide to be used/administered in the context of the invention may be a mutation of Thr (T), Asn (N), Gly (G) and/or Val (V). This means, that the amino acid residue to be replaced may be Thr (T), Asn (N), Gly (G) and/or Val (V). Preferably, the mutation is a mutation to Lys (K). This means that the different amino acid residue which replaces the amino acid residue to be replaced preferably may be Lys (K). The most preferred, however non-limiting, example of a cyclotide to be used/administered in the context of the invention, in particular of the mutated form of a cyclotide to be used/administered in the context of the invention, is T20K (SEQ ID NO: 7).

In one aspect, a cyclotide to be used/administered in the context of the invention may be selected from the group consisting of:
(i) a cyclotide comprising, or consisting of a head-to-tail cyclized form of, an amino acid sequence as depicted in any one of SEQ ID NOs: 7, 5, 4, 6 or 155;
(ii) a kalata B-type cyclotide, a Caripe-type cyclotide or a Viola-type cyclotide carrying the same mutation(s) as any one of the amino acid sequences as depicted in SEQ ID NOs: 7, 5, 4 or 6, respectively, or carrying (a) mutation(s) at (an) amino acid position(s) which correspond(s) to the amino acid position(s) which have(has) been mutated in any one of the amino acid sequences as depicted in SEQ ID NOs: 7, 5, 4 or 6, respectively;
(iii) a cyclotide comprising, or consisting of a head-to-tail cyclized form of, an amino acid sequence of a cyclotide as depicted in Tables 3, 4 or 5, wherein said amino acid sequence carries the same mutation(s) as any one of the amino acid sequences as depicted in SEQ ID NOs: 7, 5, 4 or 6, respectively, or carries (a) mutation(s) at (an) amino acid position(s) which correspond(s) to the amino acid position(s) which have(has) been mutated in any one of the amino acid sequences as depicted in SEQ ID NOs: 7, 5, 4 or 6, respectively;
(iv) a cyclotide comprising, or consisting of a head-to-tail cyclized form of, an amino acid sequence that is at least 70%, preferably at least 80%, more preferably at least 90%, even more preferably at least 95%, even more preferably at least 98% and even more preferably at least 99%, identical to an amino acid sequence as depicted in any one of SEQ ID NOs: 7, 5, 4, 6 or 155 (or as depicted in any one of Tables 3, 4, and 5), wherein said amino acid sequence carries the same mutation(s) as any one of the amino acid sequences as depicted in SEQ ID NOs: 7, 5, 4 or 6, respectively, or carries (a) mutation(s) at (an) amino acid position(s) which correspond(s) to the amino acid position(s) which have(has) been mutated in any one of the amino acid sequences as depicted in SEQ ID NOs: 7, 5, 4 or 6, respectively; and
(v) a cyclotide comprising, or consisting of a head-to-tail cyclized form of the amino acid sequence

(formula II; SEQ ID NO. 17)
Xxx₁-Leu-Pro-Val-Cys-Gly-Glu-Xxx₂-Cys-Xxx₃-Gly-
Gly-Thr-Cys-Asn-Thr-Pro-Xxx₁-Cys-Xxx₁-Cys-Xxx₁-
Trp-Pro-Xxx₁-Cys-Thr-Arg-Xxx₁,

wherein Xxx₁ is any amino acid, non-natural amino acid or peptidomimetic; Xxx₂ is any amino acid, non-natural amino acid or peptidomimetic, preferably Thr; and Xxx₃ is any amino acid, non-natural amino acid or peptidomimetic, preferably Val or Phe; or the amino acid sequence

(formula III; SEQ ID NO. 18)
Gly-Xxx₁-Ile-Pro-Cys-Xxx₂-Xxx₃-Xxx₄-Cys-Xxx₅-
Xxx₆-Xxx₇-Xxx₈-Cys-Xxx₉-Xxx₁₀-Xxx₁₁-Ala-Xxx₁₂-
Xxx₁₃-Xxx₁₄-Cys-Xxx₁₅-Cys-Xxx₁₆-Xxx₁₇-Xxx₁₈-
Xxx₁₉-Cys-Tyr-Xxx₂₀-Xxx₂₁,

wherein Xxx₁ is Val, Ala or Leu, Xxx₂ is Gly, Ser or Thr, Xxx₃ is Glu, Gly or Ser, Xxx₄ is Ser or Thr, Xxx₅ is Val, Leu or Phe, Xxx₆ is Phe or Arg, Xxx₇ is IIe or Asn, Xxx₈ is Pro or Arg, Xxx₉ is IIe, Phe, Thr or Leu, Xxx₁₀ is Ser, Thr, IIe or Val, Xxx₁₁ is Thr, Ser, Ala, Arg or Pro, Xxx₁₂ is Val, Leu or Ala, Xxx₁₃ is IIe, Leu, Phe or Val, Xxx₁₄ is Gly or Arg, Xxx₁₅ is Ser or Thr, Xxx₁₆ is Lys, Ser or Arg, Xxx₁₇ is Asn, Asp, His or Lys, Xxx₁₈ is Lys, Asn, His or Tyr, Xxx₁₉ is Val or IIe, Xxx₂₀ is Arg, Leu, Lys or Asn and/or Xxx₂₁ is Asn or Asp;
or
the amino acid sequence

(formula IIII; SEQ ID NO. 19)
Gly-Xxx₁-Xxx₂-Xxx₃-Cys-Gly-Glu-Xxx₄-Cys-Xxx₅-
Xxx₆-Xxx₇-Xxx₈-Cys-Xxx₉-Xxx₁₀-Xxx₁₁-Xxx₁₂-Cys-
Xxx₁₃-Cys-Xxx₁₄-Xxx₁₅-Xxx₁₆-Xxx₁₇-Cys-Xxx₁₈-
Xxx₁₉-Xxx₂₀,

wherein or all of Xxx₁ to Xxx₂₀ may be any amino acid, non-natural amino acid or peptidomimetic, preferably, any or all of Xxx₁ to Xxx₂₀ may be (a) conservative amino acid exchange(s) of the corresponding amino acid residue(s) of the “vitri” cyclotide as depicted in SEQ ID NO. 155,
and
wherein said amino acid sequence carries the same mutation(s) as any one of the amino acid sequences as depicted in SEQ ID NOs: 7, 5, 4 or 6, respectively, or carries (a) mutation(s) at (an) amino acid position(s) which correspond(s) to the amino acid position(s) which have(has) been mutated in any one of the amino acid sequences as depicted in SEQ ID NOs: 7, 5, 4 or 6, respectively.

Also in the context of this aspect, the preferred cyclotide is a cyclotide which carries a mutation to Lys (K), in particular at a position corresponding to (e.g. being homologous to) position 20 of the T20K cyclotide, more particular the T20K mutation itself or a corresponding mutation.

Preferred but non-limiting examples of cyclotides to be used/administered in the context of the invention are cyclotides consisting of a head-to-tail cyclized form of an amino acid sequence as defined in any of (i) to (v), supra.

In a preferred embodiment, the cyclotide to be used/administered in the context of the invention is a kalata B-type cyclotide. The kalata B-type cyclotide may be kalata B2 or a kalata B2-type cyclotide or, more preferably, kalata B1 or a kalata B1-type cyclotide. The cyclotides kalata B1 and B2 differ by only five amino acid positions (cf. SEQ ID NOs: 1 & 2; FIG. 6 of WO2013/093045; appended Table 3), namely Val to Phe (loop 2) and conservative replacements of Thr to Ser (loop 4), Ser to Thr (loop 5), Val to IIe (in loop 5) and Asn to Asp (in loop 6) in kalata B2. These substitutions have no significant structural consequences (RMSD_{backbone kB1/kB2}=0.599 Å, see FIG. 6 of WO2013/093045) and the two peptides have a similar bioactivity profile (Gruber, Toxicon 49, 2007, 561-575). The most preferred cyclotide to be used/administered in the context of the invention, however, is T20K itself (SEQ ID NO: 7).

A particular, non-limiting, example of a cyclotide to be used/administered in the context of the invention is a cyclotide comprising, or consisting of, a head-to-tail cyclized form of, an amino acid sequence as depicted in SEQ ID NO: 7, 5, 4, 6 or 155.

It is envisaged in the context of the present invention that the cyclotide to be employed is non-grafted, i.e. a non-grafted cyclotide.

The term “non-grafted” or “non-grafted cyclotide” means that the cyclotide does not include another/a further (pharmaceutically) active component, like a (pharmaceutically) active peptide or peptide epitope, which has been grafted into the cyclotide scaffold as the grafting template or grafting framework.

Thus, it is most preferred that the cyclotide to be used/administered in the context of the invention is a (naturally-occurring, native or mutated) non-grafted cyclotide, i.e. a cyclotide “per se”/“by itself” without any further (pharmaceutically) active component(s).

It is known in the art that cyclotides can act as scaffolds for other (pharmaceutically) active components, like other therapeutic peptides (see, for example, Gunasekera, 2008, J Med Chem, 51, 7697-704; Wang, ACS Chemical Biology 9, 2014, 156-63; US2010/0298528). Such grafted cyclotides, also known in the art as “grafted analogs of cyclotides” (e.g. Gunasekera loc. cit.), “bioengineered cyclic peptides” (e.g. Wang 2014 loc. cit.), and the like, i.e. cyclotides comprising a further (pharmaceutically) active component, are less preferred in the context of the present invention. Particular examples of grafted cyclotides are known to be cyclotides having at least one (+/−complete) loop between two cysteine residues be replaced by a further (pharmaceutically) active component (see, for example, Gunasekera loc. cit.; Wang 2014 loc. cit.; US2010/0298528). The “further (pharmaceutically) active component” of a grafted cyclotide is termed in the art also as “biologically active sequence” (e.g. Gunasekera loc. cit.), ““bioactive” epitope”/“(peptide) epitope” (Gunasekera loc. cit.), “(potentially) therapeutic amino acid sequence” (Wang 2014 loc. cit.), “(antigenic) peptide” (Wang 2014 loc. cit.), “graft” (US2010/0298528), and the like. Examples of such a “further (pharmaceutically) active component” are the MOG₃₅₋₅₅ epitope (Wang 2014 loc. cit.), the RGD epitope (US2010/0298528), and other epitopes which have been grafted onto a cyclotide scaffold in the context of, for example, US2010/0298528 or Gunasekera loc. cit.

This is to be seen in contrast to the cyclotides and cyclotide mutants/variants to be preferably used in the context of the present invention. Specifically, the cyclotide mutants/variants to be used in the context of the present invention are envisaged to be mutated so that no further (pharmaceutically) active component, i.e. graft, is introduced.

Thus, in the context of the present invention, the term “cyclotide” is particularly envisaged to refer to a cyclotide per se or a mutated cyclotide per se, i.e. to a non-grafted cyclotide or a non-grafted mutated cyclotide, but to exclude grafted analogs of cyclotides, bioengineered cyclic peptides and the like, i.e. grafted cyclotides. The term “cyclotide” is used accordingly also in the art.

It is possible with respect to cyclotide mutants/variants to be used/administered in the context of the invention that one or more (+/−complete) loops between two cysteine residues are replaced by (a stretch of) further amino acid residues, as long as no further (pharmaceutically) active component is introduced. The skilled person is readily in the position to distinguish between a grafted cyclotide and a non-grafted cyclotide and between a grafted cyclotide and a non-crafted cyclotide mutant/variant, respectively.

In principle, however, also grafted cyclotides may be used/administered in the context of the invention. For example, grafted cyclotides may be used/administered in combination with the disclosed kOR ligands and/or with non-grafted cyclotides.

It will be understood that for the various cyclotides to be used/administered in the context of the present invention a certain flexibility and variability in the primary sequence, i.e. the amino acid sequence backbone, is possible, as long as the overall secondary and tertiary structure of the respective peptides, which is defined by at least some fixed amino acid residues and by their spatial arrangement, is ensured (see, e.g., formulas I, II, III and IIII, supra).

Based on the teaching provided herein, the skilled person is, on the one hand, readily in the position to find out/identify corresponding mutants/variants of the cyclotides which act according to the invention. On the other hand, the skilled person is able to test whether a given cyclotide mutant/variant still has the desired function, for example at least one of the functions as described herein elsewhere. Corresponding experimental guidance for such tests, i.e. respective assays, are known in the art and are exemplarily provided and described herein and in the appended examples.

Hence, in one aspect, the present invention also relates to the use/administration of mutant or variant forms of the herein defined (native) cyclotides, in particular to the use/administration of mutant or variant forms of the cyclotides as depicted in Tables 3, 4 and 5, more particular of mutant or variant forms of kalata B2 or, preferably, kalata B1. The mutant or variant forms may be (synthetically) optimized, i.e. they may be better suited for the treatment of MS and related diseases and/or symptoms as compared to their non-mutant/non-variant form. Non-limiting examples of mutant/variant forms of cyclotides are the cyclotides as depicted in Tables 3, 4 and 5, wherein one or more of the same mutations as in any one of SEQ ID NO: 4 to 7 have been performed or one or more of corresponding (e.g. homologous) mutations at amino acid positions which correspond to (e.g. being homologous to) the amino acid positions which have been mutated in any one of SEQ ID NO: 4 to 7 have been performed.

If not mentioned differently, the term “cyclotide(s)” when used herein is envisaged to encompass also “cyclotide mutant(s)/variant(s)”.

Non-limiting examples of mutant/variant/modified cyclotides according to this invention are given in section (iv), supra or are cyclotides consisting of a head-to-tail cyclized form of an amino acid sequence as defined in section (iv), supra. Further examples of mutant/variant/modified cyclotides are cyclotides comprising an amino acid sequence selected from the group consisting of SEQ ID NOs: 4 to 7 or cyclotides consisting of a head-to-tail cyclized form of an amino acid sequence selected from the group consisting of SEQ Id NOs: 4 to 7.

As to the mutants/variants of the cyclotides it is, for example, envisaged that one or more amino acids of the respective naturally-occurring or native cyclotide are replaced by other one or more naturally-occurring or synthetic amino acid(s), respectively. In this context, it is preferred that this/these amino acid exchange(s) is/are (a) conservative amino acid exchange(s), i.e. that the replacement amino acid(s) belong(s) to the same category of amino acids than the amino acid(s) to be replaced. For example, an acidic amino acid may be replaced by another acidic amino acid, a basic amino acid may be replaced by another basic amino acid, an aliphatic amino acid may be replaced by another aliphatic amino acid, and/or a polar amino acid may be replaced by another polar amino acid.

It is particularly envisaged that the amino acid exchanges which lead to mutants/variants of the disclosed cyclotides are as such that the pattern of polarity and charge within the tertiary structure of the resulting mutant/variant still (substantially) mimics/corresponds to the three-dimensional structure of the respective cyclotide.

Further examples of mutant or variant cyclotides are kalata B-type cyclotides (for example kalata B1 or a mutant/variant thereof or kalata B2 or a mutant thereof; see also Table 3); or Caripe-type cyclotides (for example any of Caripe 1-13 or a mutant/variant thereof or Psyle E or a mutant/variant thereof; see also Table 4); or Viola-type cyclotides (for example any of Viola-type cyclotides as depicted in Table 5 or a mutant/variant thereof; see also Table 5); or cyclotides consisting of a head-to-tail cyclized form of the amino acid sequence of SEQ ID NO: 1 or 2 or of the amino acid sequence of any one of SEQ ID NO:s 20-187, having
(i) at least 1, 2, 3, 4, 5, 6, 7, 8, 9 or 10 of its acidic amino acid residues replaced by 1, 2, 3, 4, 5, 6, 7, 8, 9 or 10 different amino acid residue(s), respectively, selected from the group consisting of acidic amino acid residue;
(ii) at least 1, 2, 3, 4, 5, 6, 7, 8, 9 or 10 of its basic amino acid residues replaced by 1, 2, 3, 4, 5, 6, 7, 8, 9 or 10 different amino acid residue(s), respectively, selected from the group consisting of basic amino acid residues; and/or
(iii) at least 1, 2, 3, 4, 5, 6, 7, 8, 9 or 10 of its aliphatic amino acid residues replaced by 1, 2, 3, 4, 5, 6, 7, 8, 9 or 10 different amino acid residue(s), respectively, selected from the group consisting of aliphatic amino acid residues.

Other mutant/variant cyclotides comprise the amino acid stretch of formula II, III or IIII, but having
(i) at least 1, 2, 3, 4, 5, 6, 7, 8, 9 or 10 of the (remaining specific) acidic amino acid residues replaced by 1, 2, 3, 4, 5, 6, 7, 8, 9 or 10 different amino acid residue(s), respectively, selected from the group consisting of acidic amino acid residues;
(ii) at least 1, 2, 3, 4, 5, 6, 7, 8, 9 or 10 of the (remaining specific) basic amino acid residues replaced by 1, 2, 3, 4, 5, 6, 7, 8, 9 or 10 different amino acid residue(s), respectively, selected from the group consisting of basic amino acid residues; and/or
(iii) at least 1, 2, 3, 4, 5, 6, 7, 8, 9 or 10 of the (remaining specific) aliphatic amino acid residues replaced 1, 2, 3, 4, 5, 6, 7, 8, 9 or 10 different amino acid residue(s), respectively, selected from the group consisting of aliphatic amino acid residues.

In general, the meaning of the term “amino acid” or “amino acid residue” is known in the art and is used herein accordingly. Thereby, it is of note that when an “amino acid” is a component of a peptide/protein the term “amino acid” is used herein in the same sense than “amino acid residue”.

Particularly, an “amino acid” or “amino acid residue” as referred to herein is envisaged to be a naturally-occurring amino acid, more preferably a naturally-occurring L-amino acid. However, albeit less preferred, an “amino acid” or “amino acid residue” in context of this invention may also be a D-amino acid or a non-naturally-occurring (i.e. a synthetic) amino acid, like, for example, norleucine, ß-alanine, or selenocysteine.

Also known in the art is the meaning of the terms “acidic amino acid(s)”, “basic amino acid(s)”, “aliphatic amino acid(s)” and “polar amino acid(s)” (see, for example, Stryer, Biochemie, Spectrum Akad. Verlag, 1991, Item I. 2.). These terms are correspondingly used throughout this invention. Thereby, the particular provisos given herein with respect to the cyclotides of the invention also apply.

Particularly, the term “acidic amino acid(s)” as used herein is intended to mean an amino acid selected from the group comprising Asp, Asn, Glu, and Gln, the term “basic amino acid(s)” as used herein is intended to mean an amino acid selected from the group comprising Arg, Lys and His, the term “aliphatic amino acid(s)” as used herein is intended to mean any amino acid selected from the group comprising Gly, Ala, Ser, Thr, Val, Leu, IIe, Asp, Asn, Glu, Gln, Arg, Lys, Cys and Met, and the term “polar amino acid(s)” as used herein is intended to mean any amino acid selected from the group comprising Cys, Met, Ser, Tyr, Gln, Asn and Trp.

In a preferred embodiment, the cyclotides and mutant/variant cyclotides to be used/administered in accordance with the present invention are cyclotides having at least one of their amino acid residues corresponding to (e.g. being homologous to) Xxx₁ of formula II, preferably corresponding to (e.g. being homologous to) Xxx₁ at position 20 and/or 29 of formula II, replaced by (a) different amino acid residue(s). Likewise, the cyclotides and mutant/variant cyclotides to be used/administered in accordance with the present invention may also be cyclotides having at least one of their amino acid residues corresponding (e.g. being homologous to) to amino acid position 1, 18, 20, 22, 25 and/or 29, preferably corresponding to amino acid position 20 and/or 29, replaced by (a) different amino acid residue(s). In this context, “corresponding to” particularly means the same amino acid amino acid residue(s) and/or at the same or similar position(s). More particular, “corresponding” means being homologous. Such (a) different amino acid residue(s) may, for example, be useful for labelling the respective mutant/variant cyclotides. A non-limiting example of such (a) different amino acid residue(s) is Lys. Non-limiting examples of respective mutant/variant cyclotides are mutant/variant cyclotides comprising or consisting of (a head-to-tail cyclized form of) a amino acid sequence of SEQ ID NO: 4 to 7, wherein SEQ ID NOs. 5 or 7 are preferred; SEQ ID NO. 7 is most preferred.

In a specific aspect, the mutant/variant cyclotides to be used according to the invention are cyclotides not having replaced one or more of their amino acid residues lying between the “first” and the “second” Cys (corresponding to the “first” and “second” Cys, respectively, as depicted in formula I, supra) and/or between the “second” and the “third” Cys (corresponding to the “second” and “third” Cys, respectively, as depicted in formula I, supra).

Preferably, in such mutant/variant cyclotides none of the amino acid residues flanking the “second” Cys, in particular neither the amino acid residue next to the “second” Cys in the N-terminal direction of formula I nor the amino acid residue next to the “second” Cys in the C-terminal direction of formula I, are replaced by another amino acid residue, in particular not by an Lys or Ala residue.

It is preferred that the used/administered cyclotides and mutants/variants thereof lack sites susceptible for hydrolysis or cleaving proteases, like, for example, serum proteases. The meanings of the terms “hydrolysis” and “(serum) proteases” and the structure of the sites are well known in the art.

In a preferred aspect, in the mutant/variant cyclotides to be used/administered in accordance with the invention, in particular in the mutant/variant cyclotides more specifically defined herein elsewhere (for example, the mutant/variant cyclotides as defined in items (iv) and (i) to (iii), supra, or items (i) to (v), infra), none of the (six) Cys residues is replaced by another amino acid residue.

However, with respect to the mutants/variants of the cyclotides, one or more of the (six) Cys residues, in particular the herein defined Cys, may also be replaced by (an)other amino acid(s), as long as the replacement still leads to an individual intramolecular linkage, like that of a disulphide bond, within the cyclopeptide, i.e. to a correct dolding/mimicry of the native cyclotide. Such amino acid may, inter alia, be a non-naturally-occurring amino acid, like a non-naturally-occurring amino acid having an —SH group able to form a disulphide bond. However, it is preferred herein that a Cys, in particular a Cys given in formula I, above, is a naturally-occurring amino acid, preferably Cys itself.

It will also be acknowledged by the one skilled in the art that one or several of the amino acids forming the cyclotide to be employed according to the present invention may be modified. In accordance therewith, any amino acid as used/defined herein may also represent its modified form. For example, an alanine residue as used herein may comprise a modified alanine residue. Such modifications may, among others, be a methylation or acylation, or the like, whereby such modification or modified amino acid is preferred as long as the thus modified amino acid and more particularly the cyclotide containing said thus modified amino acid is still functionally active as defined herein. Respective assays for determining whether such a cyclotide, i.e. a cyclotide comprising one or several modified amino acids, fulfils this requirement, are known to the one skilled in the art and, among others, also described herein, particularly in the example part.

The invention also provides the use of derivatives of the disclosed cyclotides such as salts with physiologic organic and anorganic acids like HCl, H₂SO₄, H₃PO₄, malic acid, fumaric acid, citronic acid, tatratic acid, acetic acid.

It is particularly envisaged that the herein defined cyclotides, and the herein defined mutant cyclotides and variant cyclotides (see, for example, item (iv) or items (i) to (iii), supra) have at least one of the desired functions according to this invention, in particular, one (or more) of the functions as mentioned in items (i) to (v) herein below. This/these function(s) make(s) cyclotides and cyclotide mutants/variants being cyclotides well suitable for the treatment of MS and related diseases, defects and/or symptoms in accordance with the present invention.

A cyclotide or cyclotide mutant/variant to be used/administered in accordance with the invention (i.e. in combination with the ligand of the kOR) and as defined herein is envisaged to be
(i) capable of increasing (in vitro and/or in vivo) the biase factor of the ligand of the kOR as defined herein (e.g. by 5%, ≥10%, ≥15%, ≥20%, ≥30%, ≥50%, 75%, ≥100%, ≥1.5-fold, ≥2-fold, 3-fold or 5-fold);
(ii) capable of binding (in vitro and/or in vivo) to and/or activating (in vitro and/or in vivo) the kOR as defined herein (“activating”, for example, means reducing intracellular cAMP reduction, or recruitment of beta-arrestinas defined herein);
(iii) capable of acting (in vitro and/or in vivo) as an orthosteric agonist of the kOR as defined herein, for example as an orthosteric agonist of the kOR as defined herein with a low μM affinity and/or a low μM potency (for example in the range of about 0.5 μM-20 μM);
(iv) capable of acting (in vitro and/or in vivo) as an allosteric modulator (negative or, preferably, positive allosteric modulator) of the ligand of the kOR as defined herein, in particular of an endogenous ligand of the kOR (e.g. of the endogenous ligand of the kOR as defined herein); and/or
(v) not capable of inducing β-arrestin 2 recruitment and/or (capable of acting as) a biased ligand of the kOR as defined herein (e.g. no/reduced arrestin recruitment, e.g. β-arrestin 2 recruitment; less and/or slower or no internalization of the kOR; reduced chance for tolerance (cf. opioid crisis); and/or no/reduced adverse effects, such as opioid crisis/tolerance and/or dysphoria, sedation, diuresis and/or hallucinations).

In particular, a cyclotide or cyclotide mutant/variant to be used/administered in accordance with the invention (i.e. in combination with the ligand of the kOR) and as defined herein is envisaged to exhibit the function(s) of (i) or (v), supra, preferably of (i) and (v), supra.

There are examples of knottins known in the art that can enter the brain via the blood-brain barrier (e.g. scorpion derived chlorotoxin). Further, under certain circumstances (for example when there is a leaky blood-brain barrier, which may, for example, be associated with a disease (e.g. certain inflammatory or autoimmune conditions), like a disease as defined herein) cyclotides may enter the brain via the blood-brain barrier. Thus, a cyclotide or cyclotide mutant/variant to be used/administered in accordance with the invention (i.e. in combination with the ligand of the kOR) and as defined herein may be capable of penetrating the CNS (e.g. the brain or spinal cord); for example in addition to one ore more of the above functions.

Further, a cyclotide or cyclotide mutant/variant to be used/administered in accordance with the invention (i.e. in combination with the ligand of the kOR) and as defined herein may be capable of passing the blood-brain barrier; for example in addition to one ore more of the above functions.

However, cyclotides are considered in the art as usually not being capable of crossing the blood-brain barrier (or only to a low or marginal extent).

Thus, cyclotides or cyclotide mutants/variants which are not capable of penetrating the CNS (e.g. the brain or spinal cord) or passing the blood-brain barrier are preferentially be used/administered in accordance with the invention (i.e. in combination with the ligand of the kOR) and as defined herein. Such cyclotides or cyclotide mutants/variants may peripherally act in accordance with the invention. In other words, such cyclotides or cyclotide mutants/variants may function in accordance with the invention by peripheral action. Peripheral action may be reducing or inhibiting peripheral immune cell invasion and or (a) peripheral immune response(s) (like, for example CNS (e.g. the brain or spinal cord) infiltration by CD4+ and/or CD8+ T-cells).

The skilled person is readily able to test whether a given cyclotide, like a cyclotide as defined herein (e.g. by way of a structure), exhibits the relevant feature(s) in accordance with the invention, in particular the feature(s) as defined in items (i), (ii), (iii), (iv), and/or (v), supra. Respective means and methods are known in the art and are also described herein, for example in the appended examples. In this context, the skilled person can choose and/or optimize the relevant conditions, e.g. concentrations (of the cyclotide and/or kOR ligand and/or kOR). For example, the cyclotide concentration in a low micromolar range (e.g. 0.5 to 20 μM) may be appropriate in this respect.

A cyclotide which is capable of acting as an allosteric modulator (negative or, preferably, positive) of the ligand of the kOR (see item (iv), supra), may modulate (decrease or, preferably, increase) (i) the potency of the kOR ligand by (about) 1.1-, 1.2-, 1.3-, 1.5-, 1.8-, 2-, 3-, 5-, 10-, 20- or 30-fold; and/or (ii) the efficacy of the kOR ligand by (about)≥5%, 10%, 15%, 20%, 25%, 30%, 50%, 75%, 100%, 150% or 200%.

It is particularly envisaged that the capability of the cyclotide of acting as an allosteric modulator of the ligand of the kOR does not come along with a (measurable) arrestin recruitment (see also item (v), supra).

A cyclotide which is capable of binding to and/or activating the kOR as defined herein (in vitro and/or in vivo) (see item (ii), supra) may bind to the kOR with a Kᵢ in the range of, for example, (about) 0.5-10, 1-5, 2-4, 2.4-3 or 2.7±0.01-0.25 μM; and/or may activate the kOR (e.g. reducing intracellular cAMP reduction) with an EC₅₀ in the range of, for example, (about) 1-50, 5-30, 10-25, 14-20 or 17±0.11-2.2 μM.

If not indicated differently herein elsewhere, “inhibiting”, “decreasing”, “blocking”, “suppressing” or “reducing” and the like, in the context of the present invention is envisaged to mean that the initial status (for example status of demyelination, in particular of oligodendrocytes, existing CNS lesions (e.g. brain lesions), potency and/or efficacy of kOR ligands, activity of cyclotides, activity of the kOR etc.) is lowered (in vitro and/or in vivo) by, for example, at least 10%, by at least 20%, by at least 30%, by at least 50%, by at least 80%, by at least 90%, at least 95%, by at least 99% or even by 100% (in principle, the higher values of percentage are preferred). The skilled person is readily in the position to test the respective degree of “inhibiting”, “decreasing”, “blocking”, “suppressing” or “reducing” (for example by determining the status of demyelination, in particular of oligodendrocytes, existing CNS lesions (e.g. brain lesions), potency and/or efficacy of kOR ligands, activity of cyclotides etc.). Moreover, the skilled person is readily in the position to determine for a given drug (e.g. cyclotide or kOR ligand as disclosed herein) the IC₅₀ for the respective “inhibiting”, “decreasing”, “blocking”, “suppressing” or “reducing” effect/activity.

Likewise, if not indicated differently herein elsewhere, “increasing”, “inducing” or “improving”, and the like, in the context of the present invention particularly means that the initial status (for example status of (re)myelination, in particular of oligodendrocytes, potency and/or efficacy of kOR ligands, activity of cyclotides, activity of the kOR etc.) is increased (in vitro and/or in vivo) by, for example, at least 10%, by at least 20%, by at least 30%, by at least 50%, by at least 80%, by at least 90%, at least 95%, by at least 99% or by at least 100% (in principle, the higher values of percentage are preferred). In particular, “increasing” means that there is already an initial degree of activity/status (e.g. (re)myelination, potency and/or efficacy of kOR ligands, activity of cyclotides etc.) which is further “increased”. In particular, “inducing” means that there is (substantially) no initial degree of activity/status which is then “induced”. The skilled person is readily in the position to test the respective degree of “increasing”, “inducing” or “improving”, (for example by determining the status of (re)myelination, in particular of oligodendrocytes, potency and/or efficacy of kOR ligands, activity of cyclotides, etc.). Moreover, the skilled person is readily in the position to determine for a given drug (e.g. cyclotide or kOR ligand as disclosed herein) the IC₅₀ for the respective “increasing”, “inducing” or “improving” effect/activity.

If not specified differently, “induction”, “induce” or “induced” in the context of this invention means starting from a baseline which is virtually zero. “Increase”/“increased” or “enhance”/“enhanced” not necessarily means starting from a baseline which is virtually zero but may also mean starting from a level which is already above zero. For example, an induced remyelination is meant, when there initially was no remyelination activity or expression at all; an enhanced/increased remyelination is meant, when there initially was already some remyelination activity which is then further enhanced/increased.

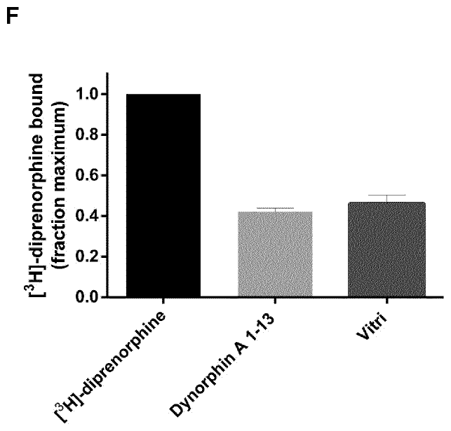

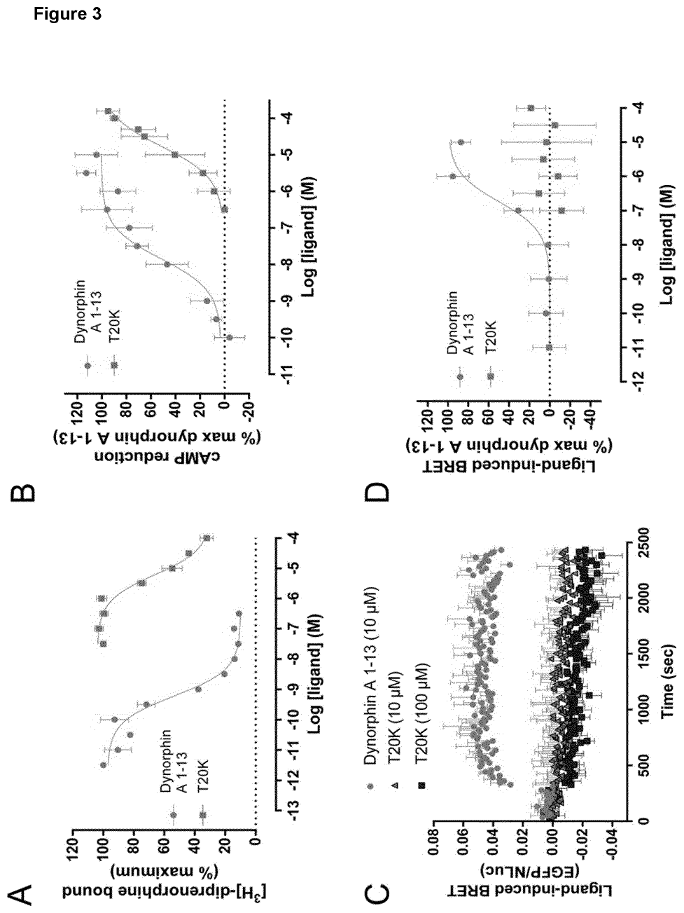

The skilled person is readily in the position to test whether a given cyclotide or cyclotide mutant/variant or kOR ligand is capable of functioning in accordance with the present invention, e.g. has one or more of the functions described herein, for example one or more of the functions defined in sections (i) to (iv), supra. For this purpose, the skilled person may, for example, rely on the assays described herein elsewhere and in the appended examples, and on respective assays as described in the art (e.g. Du loc. cit.) and referred to herein elsewhere and in the appended examples. For example, assays for testing the activation of kOR are disclosed in Du (loc. cit.). In particular, assays for testing the effect of kOR activation on remyelination (promotion of remyelination) and on stimulation of oligodendrocyte differentiation and myelination are described in Du (loc. cit.; cf. FIGS. 3, 4, and 5 thereof). Respective animal models, like mouse models, are also known in the art. These are, for example, the EAE mouse model and the cuprizone-induced demyelination mouse model (see also Du, loc. cit.).

By relying on the herein described means and methods and his common general knowledge, the skilled person is in the position to identify and isolate suitable cyclotides or cyclotide mutants/variants, for example in/from a (plant) extract. Thus, the skilled person is also able to identify and isolate not yet known cyclotides/cyclotide mutants/variants that can be used in accordance with the present invention. The use of such newly identified/isolated cyclotides in accordance with the present invention is also envisaged.

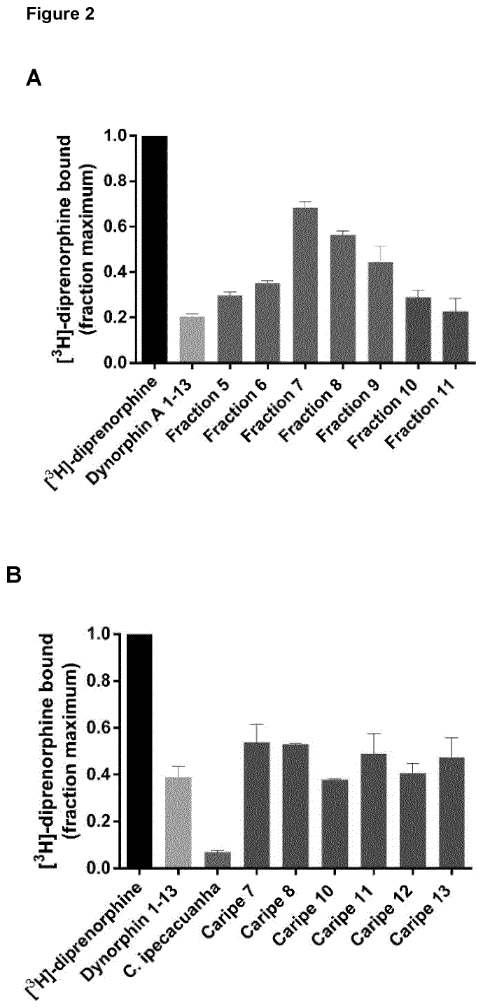

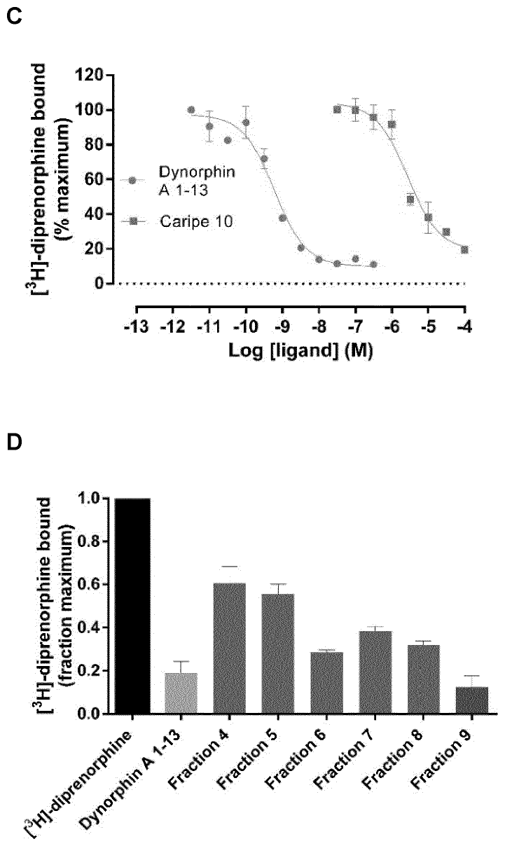

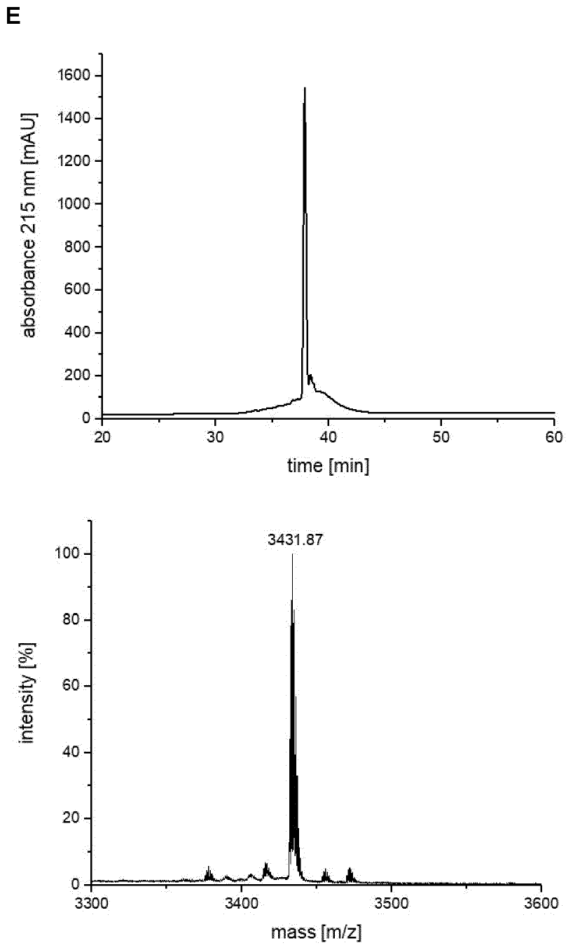

An example of such a newly isolated/identified cyclotide is the “vitri” cyclotide that has been identified in the context of the present invention (isolated from V. tricolor, cf. Example 2 and FIG. 2). The “vitri” cyclotide has been characterized by analytical RP-HPLC chromatogram and MALDI mass spectrum. Its peptide mass is about 3431.87 [m/z] (labeled as monoisotopic mass ([M+H]⁺ in FIG. 2E). In particular, the “vitri” cyclotide was purified by RP-HPLC e.g. on a Dionex Ultimate 3000 HPLC unit (Thermo-Fisher Dionex) using, for example, semipreparative (see, for example, below for details) and analytical (e.g. 250 mm×4.6 mm) Kromasil C₁₈ columns (e.g. 5 μm, 100 Å) with linear gradients of, for example, 0.1-1% min⁻¹ solvent B [e.g. 90% (vol/vol) acetonitrile, 0.1% (vol/vol) TFA in water] at flow rates of, for example, 3 mL·min⁻¹ and 1 mL·min-1, respectively.

The skilled person is readily able to identify and isolate further not yet known cyclotides/cyclotide mutants/variants that can be used in accordance with the present invention, i.e. in combination with the ligand of the kOR as defined herein, by relying on the same or similar isolation/identification means and methods.

The present invention further relates to cyclotides which can be isolated/identified by the same or similar isolation/identification means and methods as described herein and can be used in accordance with the present invention. An example of such an isolated/identified cyclotide is the “vitri” cyclotide as disclosed herein. The present invention also relates to this particular “vitri” cyclotide and mutants/variants thereof. What has been said herein elsewhere with respect to cyclotide mutants/variants applies here, mutatis mutandis.

The present invention further relates a nucleic acid molecule comprising a nucleotide sequence encoding the amino acid backbone/primary amino acid sequence of a cyclotide as disclosed and/or isolated/identified in the context of this invention; and to the respective uses of such a nucleic acid molecule. For example, such a nucleic acid molecule may comprise a nucleotide sequence as depicted in any one of SEQ ID NOs. 11, 12, 15 and 16 or a nucleotide sequence as comprised in any one of SEQ ID NOs. 11, 12, 15 and 16 and corresponding to the mature cyclotide or a nucleotide sequence which differs therefrom due to the degeneracy of the genetic code.

The meanings of the terms “nucleic acid molecule(s)”, “nucleic acid sequence(s)” and “nucleotide sequence(s)” are well known in the art and are used accordingly in the context of the present invention.

For example, when used throughout this invention, these terms refer to all forms of naturally-occurring or recombinantly generated types of nucleotide sequences and/or nucleic acid sequences/molecules as well as to chemically synthesized nucleotide sequences and/or nucleic acid sequences/molecules. These terms also encompass nucleic acid analogues and nucleic acid derivatives such as e. g. locked DNA, PNA, oligonucleotide thiophosphates and substituted ribo-oligonucleotides. Furthermore, these terms also refer to any molecule that comprises nucleotides or nucleotide analogues.

Preferably, the terms “nucleic acid molecule(s)”, “nucleic acid sequence(s)” and “nucleotide sequence(s)” and the like refer to deoxyribonucleic acid (DNA) or ribonucleic acid (RNA). The “nucleic acid molecule(s)”, “nucleic acid sequence(s)” and “nucleotide sequence(s)” may be made by synthetic chemical methodology known to one of ordinary skill in the art, or by the use of recombinant technology, or may be isolated from natural sources, or by a combination thereof. The DNA and RNA may optionally comprise unnatural nucleotides and may be single or double stranded. “Nucleic acid molecule(s)”, “nucleic acid sequence(s)” and “nucleotide sequence(s)” also refer to sense and anti-sense DNA and RNA, that is, a nucleotide sequence which is complementary to a specific sequence of nucleotides in DNA and/or RNA.

Furthermore, the terms “nucleic acid molecule(s)”, “nucleic acid sequence(s)” and “nucleotide sequence(s)” and the like may refer to DNA or RNA or hybrids thereof or any modification thereof that is known in the state of the art (see, e.g., U.S. Pat. Nos. 5,525,711, 4,711,955, 5,792,608 or EP 302175 for examples of modifications). These molecules of the invention may be single- or double-stranded, linear or circular, natural or synthetic, and without any size limitation. For instance, the “nucleic acid molecule(s)”, “nucleic acid sequence(s)” and/or “nucleotide sequence(s)” may be genomic DNA, cDNA, mRNA, antisense RNA, ribozymal or a DNA encoding such RNAs or chimeroplasts (Cole-Strauss Science 1996 273(5280) 1386-9). They may be in the form of a plasmid or of viral DNA or RNA. “Nucleic acid molecule(s)”, “nucleic acid sequence(s)” and “nucleotide sequence(s)” and the like may also refer to (an) oligonucleotide(s), wherein any of the state of the art modifications such as phosphothioates or peptide nucleic acids (PNA) are included.

The nucleic acid molecules as provided herein are particularly useful for producing a cyclic peptide of the invention, for example by a corresponding method disclosed herein.

The nucleic acid molecule as disclosed herein and described herein may be comprised in a vector.

Said vector may be a cloning vector or an expression vector, for example, a phage, plasmid, viral or retroviral vector. Retroviral vectors may be replication competent or replication defective. In the latter case, viral propagation generally will occur only in complementing host/cells. The herein disclosed nucleic acid molecule may be joined to a particular vector containing selectable markers for propagation in a host. Generally, a plasmid vector is introduced in a precipitate, such as a calcium phosphate precipitate or rubidium chloride precipitate, or in a complex with a charged lipid or in carbon-based clusters, such as fullerens. Should the vector be a virus, it may be packaged in vitro using an appropriate packaging cell line prior to application to host cells.

Preferably, the disclosed nucleic acid molecule is operatively linked to expression control sequences (e.g. within the herein disclosed vector) allowing expression in prokaryotic or eukaryotic cells or isolated fractions thereof. Expression of said polynucleotide comprises transcription of the nucleic acid molecule, preferably into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (Invitrogen), pSPORT1 (GIBCO BRL). Preferably, said vector is an expression vector and/or a gene transfer vector. Expression vectors derived from viruses such as retroviruses, adenoviruses, vaccinia virus, adeno-associated virus, herpes viruses, or bovine papilloma virus, may be used for delivery of the polynucleotides or vector of the invention into a targeted cell population. Methods which are well known to those skilled in the art can be used to construct a vector in accordance with this invention, see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the disclosed polynucleotides and vectors can be reconstituted into liposomes for delivery to target cells.

The term “isolated fractions thereof” refers to fractions of eukaryotic or prokaryotic cells or tissues which are capable of transcribing or transcribing and translating RNA from the vector. Said fractions comprise proteins which are required for transcription of RNA or transcription of RNA and translation of said RNA into a polypeptide. Said isolated fractions may be, e.g., nuclear and cytoplasmic fractions of eukaryotic cells such as of reticulocytes. Kits for transcribing and translating RNA which encompass the said isolated fractions of cells or tissues are commercially available, e.g., as TNT reticulolysate (Promega).

Again, like the disclosed nucleic acid molecules, also the disclosed vectors are particularly useful for producing a cyclic peptide of the invention, for example by a corresponding method disclosed herein.

In a further aspect, disclosed herein is a recombinant host cell comprising the nucleic acid molecule and/or the vector and/or the encoded cyclotide as disclosed herein. In context of this aspect, the nucleic acid molecule and/or the vector can, inter alia, be used for genetically engineering host cells, e.g., in order to express and isolate the amino acid backbone/primary amino acid sequence of the cyclotides disclosed herein.

Said host cell may be a prokaryotic or eukaryotic cell. The nucleic acid molecule or vector which is present in the host cell may either be integrated into the genome of the host cell or it may be maintained extra chromosomally.

The host cell can be any prokaryotic or eukaryotic cell, such as a bacterial, insect, fungal, plant, animal, mammalian or, preferably, human cell. Preferred fungal cells are, for example, those of the genus Saccharomyces, in particular those of the species S. cerevisiae, or those belonging to the group of hyphal fungi, for example several penicillia or aspergilla strains. The term “prokaryotic” is meant to include all bacteria which can be transformed or transfected with a nucleic acid molecule for the expression of an amino acid backbone/primary amino acid sequence of the cyclotides disclosed herein. Prokaryotic hosts may include gram negative as well as gram positive bacteria such as, for example, E. coli, S. typhimurium, Serratia marcescens and Bacillus subtilis. A nucleic acid molecule coding for an amino acid backbone/primary amino acid sequence of the cyclic cyclotides disclosed herein can be used to transform or transfect a host using any of the techniques commonly known to those of ordinary skill in the art. Methods for preparing fused, operably linked genes and expressing them in bacteria or animal cells are well-known in the art (Sambrook, supra). The genetic constructs and methods described therein can be utilized for expression of the above-mentioned amino acid backbone/primary amino acid sequence in, for example, prokaryotic hosts.

In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. The transformed prokaryotic hosts can be grown in fermentors and cultured according to techniques known in the art to achieve optimal cell growth. The expressed peptides can then be isolated from the grown medium, cellular lysates, or cellular membrane fractions. The isolation and purification of the microbially or otherwise expressed peptides may be by any conventional means such as, for example, preparative chromatographic separations and immunological separations such as those involving the use of monoclonal or polyclonal antibodies (Ausubel, Current Protocols in Molecular Biology, Green Publishing Associates and Wiley Interscience, N.Y. (1994)).

Like the nucleic acid molecules and the vectors as disclosed and described herein, also the corresponding host cells are particularly useful for producing a cyclotide as disclosed herein, for example by the corresponding method disclosed herein.

The skilled person is readily able to provide, i.e. synthesize, the cyclotides to be used/administered in accordance with the invention or isolate/identify them from, for example, extracts (for example biological extracts like plant, fungal, animal or microbial extracts); see, for example, the appended Examples and respective literature cited herein.

In particular, (bio-)chemically synthesizing approaches or generation of cyclotides via recombination techniques may be employed. For example, a method for producing a cyclotide may comprise the steps of
a) (i) culturing the herein disclosed recombinant host cell under conditions such that the amino acid backbone of the herein disclosed cyclotide is expressed, and recovering said amino acid backbone; or
   (ii)chemically synthesizing the amino acid backbone of the herein disclosed cyclotide (for example by solid phase peptide synthesis); and
b) cyclization of said amino acid backbone to form the herein disclosed cyclotide (for example by (native) chemical ligation).

The method for producing a cyclotide may further comprise the step of (head-to-tail) cyclization (see, for example, Grundemann, 2013 loc. cit.; Thell, loc. cit.). The method for producing a cyclotide may also (further) comprise the step of oxidative folding (see, for example, Grundemann, 2013 oc. cit.; Thell, loc. cit.).

As mentioned above, the linear peptides/amino acid backbones of the cyclotides to be produced can also be produced by recombinant engineering techniques. Such techniques are well known in the art (e. g. Sambrook, supra). As also mentioned above, by this kind of production of said linear peptides/amino acid backbones particular advantage can be taken of the herein disclosed and described nucleic acid molecules, vectors and/or host cells. The definitions correspondingly given above apply here, mutatis mutandis.

Several approaches of peptide synthesis, in particular synthesis approaches of cyclic peptides, are known in the art. (e.g. Williams, Chemical Approaches to the Synthesis of Peptides, CRC-Press 1997; Benoiton: Chemistry of Peptide Synthesis. CRC-Press, 2005). The skilled person is readily in the position to apply the prior art knowledge to the particular requirements of the disclosed method for producing cyclic peptides, based on the herein provided teaching.

This invention also relates to the use/administration of a cyclotide obtainable or obtained by the above-described approaches or method(s) in accordance with the herein provided disclosure.

The present invention further relates to a combination of
(a) a cyclotide as defined herein; and
(b) a ligand of the kOR as defined herein.

What is said herein elsewhere with respect to said cyclotide and ligand applies here, mutatis mutandis.

Particular, however non-limiting, examples of combinations of a cyclotide and a ligand of the kOR in accordance ith the invention are selected from the group consisting of
(a) T20K (SEQ ID NO. 7) and a ligand selected from the group consisting of
   (i) nalfurafine, collybolide, noribogaine, the Salvinorin A derivative B-64, triazole1.1, 6-GNTI, HS666, HS665 and mesyl salvinorin B; or
   (ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil;
(b) N29K (SEQ ID NO. 5) and a ligand is selected from the group consisting of
   (i) nalfurafine, collybolide, noribogaine, the Salvinorin A derivative B-64 (22-thiocyanatosalvinorin A (RB-64), triazole1.1 and 6-GNTI, HS666, HS665 and mesyl salvinorin B; or
   (ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil;
(c) G18K (SEQ ID NO. 4) and a ligand is selected from the group consisting of
   (i) nalfurafine, collybolide, noribogaine, the Salvinorin A derivative B-64, triazole1.1, 6-GNTI, HS666, HS665 and mesyl salvinorin B; or
   (ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil;
(d) T20K/G1K (SEQ ID NO. 6) and a ligand is selected from the group consisting of
   (i) nalfurafine, collybolide, noribogaine, the Salvinorin A derivative B-64, triazole1.1, 6-GNTI, HS666, HS665 and mesyl salvinorin B; or
   (ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil; or
(e) the vitri cyclotide (SEQ ID NO. 155) and a ligand is selected from the group consisting of
   (i) nalfurafine, collybolide, noribogaine, the Salvinorin A derivative B-64, triazole1.1, 6-GNTI, HS666, HS665 and mesyl salvinorin B;
   (ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil; or
(f) caripe 10 (SEQ ID NO. 86) and a ligand is selected from the group consisting of
   (i) nalfurafine, collybolide, noribogaine, the Salvinorin A derivative B-64, triazole1.1, 6-GNTI, HS666, HS665 and mesyl salvinorin B; or
   (ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil.

More particular, however non-limiting, examples of combinations of a cyclotide and a ligand of the kOR in accordance ith the invention are selected from the group consisting of
(a) T20K (SEQ ID NO. 7) and nalfurafine;
(b) N29K (SEQ ID NO. 5) and nalfurafine;
(c) G18K (SEQ ID NO. 4) and nalfurafine;
(d) T20K/G1K (SEQ ID NO. 6) and nalfurafine;
(e) the vitri (SEQ ID NO. 155) and nalfurafine;
(f) caripe 10 (SEQ ID NO. 86) and nalfurafine;
(g) T20K (SEQ ID NO. 7) and U50,488;
(h) N29K (SEQ ID NO. 5) and U50,488;
(i) G18K (SEQ ID NO. 4) and U50,488;
(j) T20K/G1K (SEQ ID NO. 6) and U50,488;
(k) the vitri (SEQ ID NO. 155) and U50,488;
(l) caripe 10 (SEQ ID NO. 86) and U50,488;
(m) T20K (SEQ ID NO. 7) and dynorphin A 1-13;
(n) N29K (SEQ ID NO. 5) and dynorphin A 1-13;
(o) G18K (SEQ ID NO. 4) and dynorphin A 1-13;
(p) T20K/G1K (SEQ ID NO. 6) and dynorphin A 1-13;
(q) the vitri (SEQ ID NO. 155) and dynorphin A 1-13;
(r) caripe 10 (SEQ ID NO. 86) and dynorphin A 1-13;
(s) T20K (SEQ ID NO. 7) and difelikefalin;
(t) N29K (SEQ ID NO. 5) and difelikefalin;
(u) G18K (SEQ ID NO. 4) and difelikefalin;
(v) T20K/G1K (SEQ ID NO. 6) and difelikefalin;
(w) the vitri (SEQ ID NO. 155) and difelikefalin;
(x) caripe 10 (SEQ ID NO. 86) and difelikefalin;
(y) T20K (SEQ ID NO. 7) and nalbuphine;
(z) N29K (SEQ ID NO. 5) and nalbuphine;
(aa) G18K (SEQ ID NO. 4) and nalbuphine;
(ab) T20K/G1K (SEQ ID NO. 6) and nalbuphine;
(ac) the vitri (SEQ ID NO. 155) and nalbuphine;
(ad) caripe 10 (SEQ ID NO. 86) and nalbuphine;
(ae) T20K (SEQ ID NO. 7) and pentasozin;
(af) N29K (SEQ ID NO. 5) and pentasozin;
(ag) G18K (SEQ ID NO. 4) and pentasozin;
(ah) T20K/G1K (SEQ ID NO. 6) and pentasozin;
(ai) the vitri (SEQ ID NO. 155) and pentasozin;
(aj) caripe 10 (SEQ ID NO. 86) and pentasozin;
(ak) T20K (SEQ ID NO. 7) and pethidine;
(al) N29K (SEQ ID NO. 5) and pethidine;
(am) G18K (SEQ ID NO. 4) and pethidine;
(an) T20K/G1K (SEQ ID NO. 6) and pethidine;
(ao) the vitri (SEQ ID NO. 155) and pethidine;
(ap) caripe 10 (SEQ ID NO. 86) and pethidine;
(aq) T20K (SEQ ID NO. 7) and sulfentanil;
(ar) N29K (SEQ ID NO. 5) and sulfentanil;
(as) G18K (SEQ ID NO. 4) and sulfentanil;
(at) T20K/G1K (SEQ ID NO. 6) and sulfentanil;
(au) the vitri (SEQ ID NO. 155) and sulfentanil; and
(av) caripe 10 (SEQ ID NO. 86) and sulfentanil.

The present invention further relates to a pharmaceutical composition comprising the combination of the invention or the cyclodide and kOR ligand as described herein, and, optionally a pharmaceutically acceptable carrier.

The present invention further relates to a use of a cyclotide as defined herein for
(i) reducing adverse effects of a ligand of the kOR as defined herein (in particular of a full (“unbiased”) kOR agonist as defined herein); and/or
(ii) increasing the efficacy and/or potency of a ligand of the kOR as defined herein (in particular of a full (“unbiased”) kOR agonist as defined herein).

The present invention further relates to a kit/kit of contents/kit of parts, said kit comprising (a combination of)
(a) a cyclotide as defined herein; and
(b) a ligand of the kOR as defined herein.

Further, in the context of the kit/kit of contents/kit of parts of the invention, said (combination of)
(a) cyclotide; and
(b) ligand of the kOR may, (separately and independently from said kit), be for use in
(i) treating MS (e.g. treatment by therapy or prophylactic/preventive treatment);
(ii) remyelination (i.e. its induction or increase), in particular of oligodendrocytes, and/or improving (e.g. reducing or curing/healing) CNS lesions (e.g. brain lesions);
(iii) preventing or reducing demyelination (in particular of oligodendrocytes) and/or preventing the formation of CNS lesions (e.g. brain lesions) and/or reducing existing CNS lesions (e.g. brain lesions); and/or
(iv) treating (e.g. treatment by therapy or prophylactic/preventive treatment) pain, in particular neuropathic pain (e.g. central or peripheral neuropathic pain) and/or pain resulting from/coming along with MS.

The present invention further relates to a pharmaceutical composition as part of a kit/kit of contents/kit of parts of the invention, wherein the (combination of)
(a) cyclotide as defined herein; and
(b) ligand of the kOR as defined herein or said pharmaceutical composition, may (separately and independently from said kit), be for use in
(i) treating MS (e.g. treatment by therapy or prophylactic/preventive treatment);
(ii) remyelination (i.e. its induction or increase), in particular of oligodendrocytes, and/or improving (e.g. reducing or curing/healing) CNS lesions (e.g. brain lesions);
(iii) preventing or reducing demyelination (in particular of oligodendrocytes) and/or preventing the formation of CNS lesions (e.g. brain lesions) and/or reducing existing CNS lesions (e.g. brain lesions); and/or
(iv) treating (e.g. treatment by therapy or prophylactic/preventive treatment) pain, in particular neuropathic pain (e.g. central neuropathic pain) and/or pain resulting from/coming along with MS.

The present invention further relates to a kit/kit of contents/kit of parts comprising a pharmaceutical composition comprising (a combination of)
(a) a cyclotide as defined herein; and
(b) a ligand of the kOR as defined herein, wherein said pharmaceutical composition and/or said (a combination of)
(a) cyclotide; and
(b) ligand of the kOR may, (separately and independently from the kit), be for use in
(i) treating MS (e.g. treatment by therapy or prophylactic/preventive treatment);
(ii) remyelination (i.e. its induction or increase), in particular of oligodendrocytes, and/or improving (e.g. reducing or curing/healing) CNS lesions (e.g. brain lesions);
(iii) preventing or reducing demyelination (in particular of oligodendrocytes) and/or preventing the formation of CNS lesions (e.g. brain lesions) and/or reducing existing CNS lesions (e.g. brain lesions); and/or
(iv) treating (e.g. treatment by therapy or prophylactic/preventive treatment) pain, in particular neuropathic pain (e.g. central or peripheral neuropathic pain) and/or pain resulting from/coming along with MS.

The kit/kit of contents/kit of parts or the pharmaceutical composition according to the invention may be for use in
(i) treating MS (e.g. treatment by therapy or prophylactic/preventive treatment);
(ii) remyelination (i.e. its induction or increase), in particular of oligodendrocytes, and/or improving (e.g. reducing or curing/healing) CNS lesions (e.g. brain lesions);
(iii) preventing or reducing demyelination, in particular of oligodendrocytes, and/or preventing the formation of CNS lesions (e.g. brain lesions) and/or reducing existing CNS lesions (e.g. brain lesions); and/or
(iv) treating (e.g. treatment by therapy or prophylactic/preventive treatment) pain, in particular neuropathic pain (e.g. central or peripheral neuropathic pain) and/or pain resulting from/coming along with MS.

In the context of the kit/kit of contents/kit of parts of the invention, and the respective pharmaceutical compositions, but also in the context of the pharmaceutical compositions in general as described herein, the cyclotide and kOR ligand, or combination thereof, may be contained in a single container or vial or, preferably, in two different containers or vials.

In accordance with the present invention, the disclosed pharmaceutical composition or cyclotide and kOR ligand, or combination thereof, can/will be administered in a pharmaceutically/therapeutically effective dose. This means that a pharmaceutically/therapeutically effective amount of each compound (active ingredient) which is to be administered is reached. Preferably, a pharmaceutically/therapeutically effective dose refers to that amount of the compound administered that, for example, produces amelioration (of symptoms) of the disease, an improved condition and/or a prolongation of survival of a subject. This can be determined by the one skilled in the art doing routine testing.

The dosage regimen of the compounds to be administered in accordance with the present invention will be determined by the attending physician and clinical factors. As is well known in the medical arts, dosages for any one patient depends upon many factors, including the patient's size, body surface area, age, the particular compound to be administered, sex, time and route of administration, general health and/or other drugs being administered concurrently. A person skilled in the art is aware of and is able to test the relevant doses of the compounds to be medically applied in accordance with the present invention.

In accordance with the invention, the cyclotide and/or kOR ligand may, for example, be administered at a dose in a range of 10 μg/kg BW to 100 mg/kg BW, preferably in a range of 200 μg/kg BW to 60 mg/kg BW, more preferably in a range of 400 μg/kg BW to 40 mg/kg BW, more preferably in a range of 600 μg/kg BW to 20 mg/kg BW, and even more preferably in a range of 800 μg/kg BW to 10 mg/kg BW. Other preferred doses are in a range of up to 20 mg/kg BW.

Of course, the doses may vary depending on the administration scheme and/or administration route. For example, if administered i.v., the doses are usually lower as compared to a p.o. administration. Non-limiting examples of possible doses in the context of p.o. administration are doses in a range of 10 μg/kg BW to 100 mg/kg BW, preferably in a range of 200 μg/kg BW to 60 mg/kg BW, more preferably in a range of 400 μg/kg BW to 40 mg/kg BW. Non-limiting examples of possible doses in the context of i.v. administration are doses in a range of 600 μg/kg BW to 20 mg/kg BW, preferably in a range of 800 μg/kg BW to 10 mg/kg BW. Doses in the range of up to 20 mg/kg BW may, for example, be appropriate and safe in the context of all three, p.o., i.p. and i.v. administration.

A dose/doses in accordance with the invention may be administered on a daily, weekly or monthly basis. The cyclotide and/or kOR ligand may be administered in form of 1 or more single doses; in particular, 1, 2, 3, 4 or 5 single doses (e.g. per day, week, month). A particular, however non-limiting, example is the administration of a single dose (of up to) 3 times a week. Continuous administration is also envisaged in the context of the invention. The cyclotide and/or kOR ligand may, for example, be administered intraperitoneally or orally. Thus, in the context of a preferred, however non-limiting, embodiment, cyclotide and/or kOR ligand, or pharmaceutical composition comprising them, is administered i.v., i.p. or orally, is formulated for i.p. or oral administration and/or comprises a pharmaceutically acceptable carrier for i.p. or oral administration. A non-limiting example of a particular administration scheme are 3 single intraperitoneal injections of up to 10 mg/kg BW at weekly intervals, for example in PBS. Another non-limiting example of a particular administration scheme are 3 single oral administrations of up to 50 mg/kg BW at weekly intervals, for example in PBS. Further possible administration schemes are described herein elsewhere.

The doses/dosage regimens for the cyclotide and the kOR ligand to be employed in accordance with the invention may be the same or similar or different. In particular, the dose of the cyclotide may be the same than the dose of the kOR ligand. The dose of the cyclotide (e.g. a dose as described above) may also be higher or lower than the dosis of the kOR ligand (e.g. a dose as described above). For example, the dose of the cyclotide (e.g. a dosis as described above) may be ≥1.1-, 1.2-, 1.5-, 2- or 3-fold higher or lower than the dose of the kOR ligand (e.g. a dose as described above). A similar dose may be a dose which differs by at most 50%, 40%, 30%, 20%, 10%, 5% or 3%.

The herein described pharmaceutical composition or combination may also comprise one or more of each, cyclotide and/or kOR ligand. Hence, in a further specific embodiment, the herein described pharmaceutical composition or combination may comprise at least two, three, four or five of each, cyclotide and/or kOR ligand.

The herein described cyclotide and kOR ligand may be administered together, i.e. simultaneously, but at the same or different administration sites or by the same or different administration routes or they may be administered subsequently, at the same or different administration sites or by the same or different administration routes. In the latter case, the cyclotide may be administered first, followed by a subsequent administration of the kOR ligand, or the kOR ligand may be administered first, followed by a subsequent administration of the cyclotide.

In one specific embodiment, the herein described pharmaceutical composition may further comprise one or more additional active agents (in addition to the cyclotide(s) and kOR ligand(s)). Likewise, the herein described (pharmaceutical composition/combination comprising the) cyclotide(s) and kOR ligand(s) may be co-administered with one or more additional active agents. The herein described cyclotide(s) and kOR ligand(s) may also be used/administered in the context of a combination therapy or co-therapy, e.g. with or without one or more additional active agent(s). Preferably, this (these) additional active agent(s) is (are) not (a) “graft(s)”, i.e. not a part of a grafted form of a cyclotide, but is (are) independently comprised in the pharmaceutical composition. In another specific embodiment, the additional active agent(s) is (are) administered separately temporary and/or spatially. The herein described (pharmaceutical composition/combination comprising the) cyclotide(s) and kOR ligand(s) may be administered together with one or more additional active agent(s), i.e. prior, simultaneously or subsequently with respect to the administration of the additional active agent(s). Non-limiting examples of one or more additional active agent(s) may be selected from the group consisting of KOR liagnds (e.g. https://www.guidetopharmacology.org/GRAC/ObjectDisplayForward?objectId=318&familyId=50&familyType=GPCR) or MS therapeutics (e.g. https://pubmed.ncbi.nlm.nih.gov/3031527-treatment-of-multiple-sclerosis-success-from-bench-to-beside/?from_term=tintore+2019&from_pos=1; Tintore, Nat Rev Neurol, 15(1), 2019, 53-8).

In one embodiment, the pharmaceutical composition of the present invention may comprise, or be in form of, an (native) extract, in particular a cyclotide-containing (native) plant extract. Non-limiting examples of plants from which such an extract may be obtained are Oldenlandia affinis, Carapichea ipecacuanha (in particular the Radix lpecacuanhae), plants from the Violaceae family (e.g. Viola sp., preferably V. odorata and V. tricolor), Squash species (Cucurbitaceae family, e.g. Cucurbita pepo), Ecballium species, legume species (Fabaceae family), Psychotria species (Rubiaceae family; for example Psychotria polyphlebia, P. poeppigiana, P. chiapensis, P. borucana, P. buchtienii, P. pillosa, P. mortomiana, P. deflexa, P. makrophylla, P. elata, P. solitudinum, P. capitata), Bryonia alba, Momordica charantia, Strophantus kombe, Helianthus annuus, Sambuca nigra, Amaranthus sp. like Amaranthus caudatus, Beta vulgaris, Rauwolfia serpentina, Morinda citrifolia, Moringa oleifera, Salix alba, Salix purpurea and Cajanus cajan. The kOR ligand may be added to (such) a cyclotide-containing extract or may be co-administered with (such) a cyclotide-containing extract.

Beside their amino acid backbone, the cyclotides to be used in accordance with the invention may further comprise or be coupled with (e.g. have covalently bound) (a) further substituent(s), like labels, further active agents, anchors (like proteinaceous membrane anchors), tags (like HIS tags). The substituent(s) may be bound covalently or non-covalently to the cyclotides, and directly or via linkers. One particular site by which (a) further substituent(s) could be coupled to the cyclotide is a K residue, for example a K residue corresponding to (e.g. being homologous to) the 20K residue of the particular cyclotide T20K. The skilled person is readily in the position to find out appropriate linkers to be employed in this context. Moreover, appropriate substituents and methods for adding them to a cyclotide are known to or can be established by those of ordinary skill in the art.

Examples of labels include, inter alia, fluorochromes (like fluorine-18, fluorescein, rhodamine, Texas Red, etc.), enzymes (like horse radish peroxidase, β-galactosidase, alkaline phosphatase), radio/radioactive isotopes (like 32P, 33P, 35S, 1251 or 1231, 1351, 1241, 11C, 150), biotin, digoxygenin, colloidal metals, chemi- or bioluminescent compounds (like dioxetanes, luminol or acridiniums). One non-limiting example of a label that may be bound to the cyclotide is a fluorochrome, like a FRET fluorochrome, for example a GFP, YFP or CFP variant (e.g. GFP, YFP, CFP, eGFP, EYFP or ECFP). A variety of techniques are available for labeling biomolecules, and comprise, inter alia, covalent coupling of enzymes or biotinyl groups, phosphorylations, biotinylations, random priming, nick-translations, tailing (using terminal transferases). Such techniques are, e.g., described in Tijssen, “Practice and theory of enzyme immunoassays”, Burden and von Knippenburg (Eds), Volume 15 (1985); “Basic methods in molecular biology”, Davis L G, Dibmer M D, Battey Elsevier (1990); Mayer, (Eds) “Immunochemical methods in cell and molecular biology” Academic Press, London (1987); or in the series “Methods in Enzymology”, Academic Press, Inc. Corresponding detection methods comprise, but are not limited to, autoradiography, fluorescence microscopy, direct and indirect enzymatic reactions, etc.

The cyclotides as described and defined herein, in particular the above-described labelled cyclotides, may be employed in biodistribution studies, i.e. studies resulting in a pattern of distribution of the cyclotide, for example in an animal or, preferably a human subject/patient. For example, such biodistribution studies may comprise imaging by single-photon or PET imaging devices. Respective means and methods are known in the art (e.g. the IVIS® Spectrum In Vivo Imaging System of PerkinElmer).

Examples of further (active) agents which may be coupled to a cyclotide to be used/administered include, inter alia, antibodies as specific cell penetrating peptides (e.g. shuttle peptides for directed targeting to cells, tissues etc., e.g. brain).

In the context of the invention, “treating”/“treatment” is generally envisaged to encompass both, therapy and prevention/prophylactic treatment. Therapy may result in amelioration or even in curing/healing The cyclotide and kOR ligand to be used/administered in accordance with the invention may be comprised in (a) pharmaceutical composition(s). They me be comprised in one and the same pharmaceutical composition (i.e. in combination). Each of them may also be comprised separately and independently in different pharmaceutical compositions. Further, each of the cyclotide and kOR ligand, or both, may be comprised in the respective pharmaceutical composition(s) as the sole active ingredient(s), or together with (an)other active ingredient(s).

The pharmaceutical composition of the invention may comprise a pharmaceutically acceptable carrier, excipient or diluent.

A carrier optionally comprised in the pharmaceutical composition of the invention or to be administered together with the pharmaceutical composition or with the cyclotide and kOR ligand of the invention may particularly be a pharmaceutically acceptable carrier, excipient or diluent.

Such carriers are well known in the art. The skilled person is readily in the position to find out such carriers which are suitable to be employed in accordance with the present invention.

Pharmaceutically acceptable carriers/excipients/diluents that may be used in the formulation of the pharmaceutical compositions comprising the active compounds as defined herein (or a salt thereof) may generally comprise carriers, vehicles, diluents, solvents such as monohydric alcohols such as ethanol, isopropanol and polyhydric alcohols such as glycols and edible oils such as soybean oil, coconut oil, olive oil, safflower oil cottonseed oil, oily esters such as ethyl oleate, isopropyl myristate; binders, adjuvants, solubilizers, thickening agents, stabilizers, disintergrants, glidants, lubricating agents, buffering agents, emulsifiers, wetting agents, suspending agents, sweetening agents, colourants, flavours, coating agents, preservatives, antioxidants, processing agents, drug delivery modifiers and enhancers such as calcium phosphate, magnesium state, talc, monosaccharides, disaccharides, starch, gelatine, cellulose, methylcellulose, sodium carboxymethyl cellulose, dextrose, hydroxypropyl-ß-cyclodextrin, polyvinylpyrrolidone, low melting waxes, ion exchange resins. These and other suitable pharmaceutically acceptable carriers/excipients are described in Remington's Pharmaceutical Sciences, 15^{th} Ed., Mack Publishing Co., New Jersey (1991).

Administration of the pharmaceutical composition or the cyclotide(s) in accordance with this invention may be effected by different ways. Such may be, for example, per-oral, intravenous, intraarterial, intraperitoneal, intravesical intranodal or subcutaneous administrations or administration by inhalation as well as transdermal administration. Other examples are parenteral, such as subcutaneous, intravenous, intramuscular, intraperitoneal, intranodal, intrathecal, transdermal, transmucosal, transpulmonal subdural administrations, local or topical administrations and administrations via iontopheresis, sublingual administrations, administrations by inhalation spray or aerosol or rectal administrations, and the like.

In particular, for patients and/or for particular medical uses, particular administration routes like blood infusion (e.g. intravenous infusion), rectal administration (e.g. in form of enemas or suppositories) or topical administration routes may be indicated.

In the following, several non-limiting administration schemes and the use of correspondingly suitable pharmaceutically acceptable carrier are described.

For an administration of the pharmaceutical composition or the cyclotide(s) and kOR ligand(s) in accordance with this invention via subcutaneous (s.c.) or intravenous (i.v.)/intraarterial (i.a.) injection, cyclotides (or encoding sequences) may be formulated in aqueous solution, preferably in physiologically compatible buffers such as Hank's solution, Ringer's solution, or physiologically saline buffer. For transmucosal and transpulmonal administration, penetrants appropriate to the barrier to be permeated are used in the formulation. Such penetrants are generally known in the art.

The use of pharmaceutical acceptable carriers to formulate the cyclotide(s) and kOR ligand(s) into dosages or pharmaceutical compositions suitable for systemic, i.e. intravenous/intraarterial, intranodal or subcutaneous administration is within the scope of the present invention. With proper choice of carrier and suitable manufacturing practice, the compositions of the present invention, in particular those formulated as solutions, may be administered parenterally, such as by intravenous injection. The compounds can be readily formulated using pharmaceutically acceptable carriers well known in the art into dosages suitable for subcutaneous or oral administration. Such carriers enable the compounds according to the present invention to be formulated as tablets, pills, capsules, dragees, liquids, gels, syrups, slurries, suspensions and the like, in particular for oral ingestion by a subject to be treated.

Compounds according to the present invention, or medicaments or pharmaceutical compositions comprising them, intended to be administered intracorporally/intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered near the cell surface. Delivery systems involving liposomes are disclosed in U.S. Pat. No. 4,880,635 to Janoff et al. The publications and patents provide useful descriptions of techniques for liposome drug delivery.

Pharmaceutical compositions comprising a compound according to the present invention for parenteral and/or subcutaneous administration include aqueous solutions of the active compound(s) in water-soluble form. Additionally, suspensions of the active compounds may be prepared as appropriate oily injection suspensions. Suitable lipophilic solvents or vehicles include fatty oils such as sesame oil or castor oil, or synthetic fatty acid esters, such as ethyl oleate or triglycerides, or liposomes. Aqueous injections suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions and to allow for a constantly slow release of the substance in the organism.

A “patient”/“subject” for the purposes of the present invention, i.e. to whom (a) pharmaceutical composition(s) or cyclotide(s) and kOR ligand(s) according to the present invention is/are to be administered and/or who suffers from the disease or disorder and/or symptom(s) as defined and described herein, includes both humans and animals, as well as other organisms. Thus the compositions and methods of this invention are applicable to or in connection with both, human therapy and veterinary applications, including treating and preventing procedures and methods. In the preferred embodiment the patient/subject is a mammal, and in the most preferred embodiment the patient/subject is human.

The pharmaceutical composition, active ingredients, combination, kit/kit of contents/kit of parts etc. of the invention may be comprised in, may be a part of, or may be a medical device, a medicinal product packaging or pharmaceutical composition packaging. Such device or packaging may, for example, comprise or be (a) vial(s)/(a)container(s), (a) syringe(s) or a blister pack. The active ingredients, and pharmaceutical composition(s), respectively, may be comprised in the device or packaging separately and independently (e.g. in different vials/containers or syringes or blisters), or they may be comprised in combination (e.g. in the same vial/containers or syringe or blister).

The present invention further relates to a method of producing a pharmaceutical composition, e.g. for treating MS and/or related diseases and/or symptoms, said method comprising the step of mixing
(i) a cyclotide and/or kOR ligand as defined herein; or
(ii) a cyclotide and/or kOR ligand screened for, selected, produced, isolated or identified as described herein
   with a pharmaceutically acceptable carrier, for example a pharmaceutically acceptable carrier, excipient or diluent as defined herein elsewhere.

The method for producing a pharmaceutical composition may comprise the step of mixing
(i) a cyclotide as defined herein;
(ii) a kOR ligand as defined herein; and
(iii) a pharmaceutically acceptable carrier, for example a pharmaceutically acceptable carrier/excipient/diluents as defined herein elsewhere.

Any of the pharmaceutical compositions, active ingredients, combinations, kits etc. of the invention may be provided together with an instruction manual or instruction leaflet. The instruction manual/leaflet may comprise guidance for the skilled person/attending physician how to treat or prevent a disease, disorder or symptom as described herein in accordance with the invention, in particular MS and related diseases, defects and/or symptoms. In particular, the instruction manual/leaflet may comprise guidance as to the herein described mode of administration/administration regimen (for example route of administration, dosage regimen, time of administration, frequency of administration). In principle, what has been said herein elsewhere with respect to the mode of administration/administration regimen may be comprised in the instruction manual/leaflet.

The present invention is further described by reference to the following non-limiting figures and examples.

The Figures show:

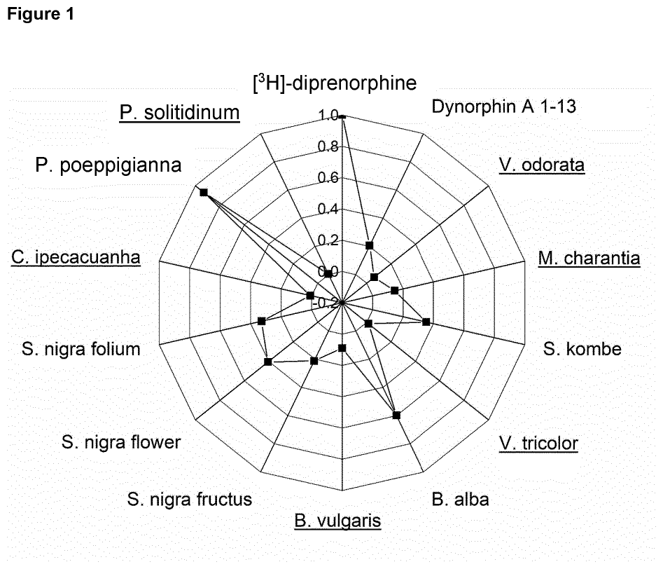

FIG. 1. Binding effects of cysteine-rich plant extracts on the kOR. Data show the fraction of binding at test concentrations of 300 μg/mL. Data presented are obtained from two independent experiments each performed in duplicate. Specific binding was calculated by subtracting the non-specific from the total bound and normalized to 1.0 (1 nM [³H]-diprenorphine). Dynorphin A 1-13 (10 nM) was used as positive control. Plant extracts colored in red showed the most pronounced binding effect.

FIG. 2. Receptor pharmacology of plant extracts of C. ipecacuanha and V. tricolor at the kOR. A) Binding data were obtained by measuring the displacement of radioactive [³H]-diprenorphine (1 nM) by peptide-enriched fractions of C. ipecacuanha (300 μg/mL) and control dynorphin A 1-13 (10 nM) (n=2). B) Displacement binding of isolated caripe peptides (10 μM) and dynorphin A 1-13 (10 nM) (n=2) as well as C) concentration-response curve of caripe 10 in HEK293 cell membranes stably expressing the kOR (n=3). The Kᵢ value of caripe 10 and dynorphin A 1-13 was calculated to be 1 μM and 280 μM, respectively. D) Displacement binding of radiolabeled [³H]-diprenorphine by peptide-enriched fractions of Viola tricolor (300 μg/mL) and positive control dynorphin A 1-13 (10 nM) (n=2). E) Analytical RP-HPLC chromatogram and MALDI mass spectrum of a vitri peptide isolated from V. tricolor. Peptide mass (3431.87) is labeled as monoisotopic mass ([M+H]+). F) Isolated vitri peptide (100 μg/mL) was tested for competing 1 nM of radioligand. Dynorphin A 1-13 (10 nM) served as positive control (n=2). Specific binding was calculated by subtracting the non-specific from the total bound and normalized to 1.0 (fraction) or 100% (percentage). 1.0 or 100% refer to approximately 5-7 pmoles of ligand bound per milligram of membrane. Data are shown as mean±SD and concentration-response curves were fitted by nonlinear regression (sigmoidal, three-parameters, Hill slope of 1).

FIG. 3. Receptor pharmacology of [T20K]-kalata B1 at the kOR. Displacement radioligand binding of radioactive diprenorphine (1 nM) by [T20K]-kalata B1 in HEK293 cell membranes stably expressing the kOR (n=2). Specific binding was calculated by subtracting the non-specific from the total bound and normalized to 100% (5-7 pmol/mg protein). B) Functional cAMP assay of [T20K]-kalata B1 in HEK293 cells stably expressing the kOR (n=4). Cells were treated with indicating concentrations of [T20K]-kalata B1 for 30 min at 37° C. Data were normalized to percentage of maximal activation, detected at the highest endogenous ligand concentration. C) BRET was monitored between Nano-Luciferase (NLuc) and EGFP introduced at the C-terminus of the kOR (the kOR-EGFP) and the β-arrestin 2 (β-arrestin 2-NLuc). HEK293 cells co-expressing the kOR-EGFP and β-arrestin 2-NLuc were stimulated by dynorphin A 1-13 (10 μM) and T20K (10 and 100 μM) 5 min after addition of the luciferase substrate (furimazine). The results are shown as differences in the BRET signals in the presence and absence of agonist and are expressed as the mean value±SD (n=3). D) Concentration response curves of dynorphin A 1-13 and T20K (n=3). Ligands were incubated for 5 min and following an endpoint measurement of bioluminescence was performed. Ligand-promoted BRET was calculated as: (emission EGFP ligand/emission NLuc ligand)−(emission EGFP HBSS/emission NLuc HBSS). Data were normalized to maximal activation of dynorphin 1-13. Data are shown as mean±SD and fitted by nonlinear regression (sigmoidal, three parameters, Hill slope of 1).

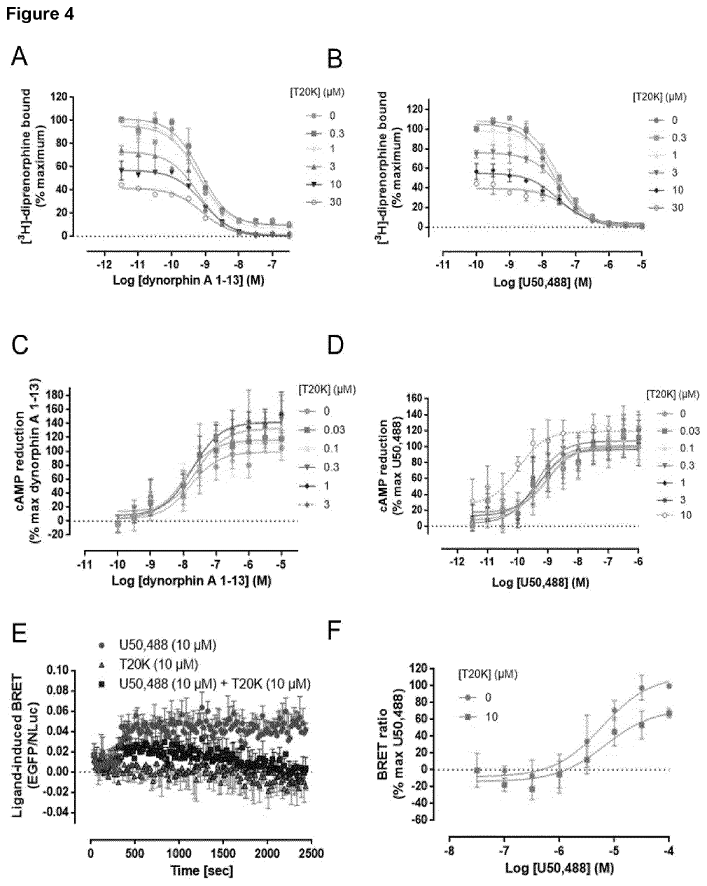

FIG. 4. [T20K]-kalata B1 acts as an allosteric modulator of the kOR. A) Displacement Displacement radioligand binding of radioactive diprenorphine (1 nM) by co-incubation of varying concentrations of [T20K]-kalata B1 and dynorphin A 1-13 or B) U50,488 in HEK293 cell membranes stably expressing the kOR (n=2). Specific binding was calculated by subtracting the non-specific from the total bound and normalized to 100% (5-7 pmol/mg protein). C) Functional cAMP assay of [T20K]-kalata B1 in combination with dynorphin A 1-13 or D) U50,488 in HEK293 cells stably expressing the kOR (n=5). Cells were incubated with [T20K]-kalata B1 for 30 min at 37° C. followed by incubation of dynorphin A 1-13 or U50,488 for another 30 min at 37° C. Data were normalized to percentage of maximal activation, detected at the highest endogenous ligand concentration. E) BRET was monitored between Nano-Luciferase (NLuc) and EGFP introduced at the C-terminus of the kOR (the kOR-EGFP) and the β-arrestin 2 (3-arrestin 2-NLuc). HEK293 cells co-expressing the kOR-EGFP and β-arrestin 2-NLuc were stimulated by U50,488 (10 μM) and T20K (10 μM) 5 min after addition of the luciferase substrate (furimazine). The results are shown as differences in the BRET signals in the presence and absence of agonist and are expressed as the mean value±SD (n=3). F) Concentration response curves of U50,488 and T20K (n=4). Ligands were incubated for 5 min and following an endpoint measurement of bioluminescence was performed. Ligand-promoted BRET was calculated as: (emission EGFP ligand/emission NLuc ligand)−(emission EGFP HBSS/emission NLuc HBSS). Data were normalized to maximal activation of U50,488. Data are shown as mean±SD and fitted by nonlinear regression (sigmoidal, three parameters, Hill slope of 1).

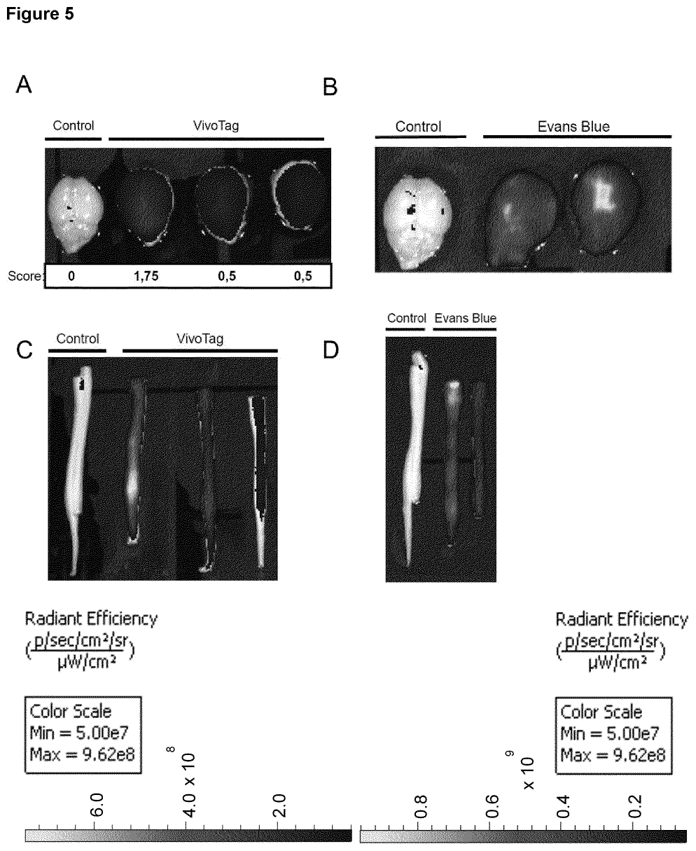

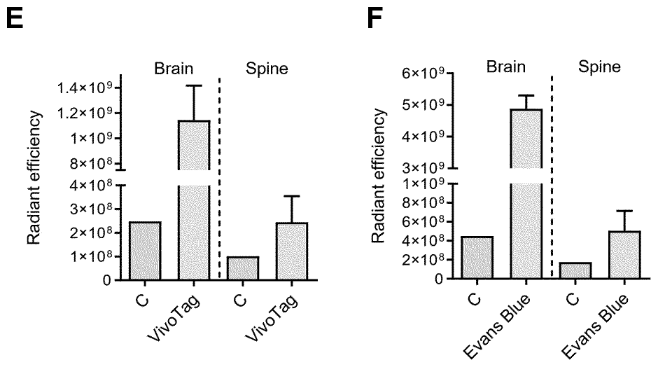

FIG. 5. Biodistribution of VivoTag-labeled [T20K]-kalata B1. [T20K-VivoTag]-kB1 (5 mg/kg) was injected i.p. into EAE mice. Biodistribution of the labeled peptide was monitored at indicated disease scores (0.5 and 1.75) by using the IVIS. 4h post-injection organs were scanned for fluorescence intensity A) [T20K-VivoTag]-kB1 and B) Evans Blue dye accumulate in the brain as well as C), D) in the spine. Quantification of E) [T20K-VivoTag]-kB1 and F) Evan Blue dye was executed using the IVIS Living Image software.

FIG. 6. Sequence diversity of cyclotides. Cyclotides identified in A) O. affinis, B) C. ipecacuanha and C) V. tricolor constitute a growing niche for discovering novel the kOR ligands as potential treatment for MS. Sequence alignment and sequence diversity wheels have been generated using tools available at the http://www.cybase.org.au/.

FIG. 7. Synthesis of cyclotides. Cyclotides were assembled as linear precursors using Fmoc chemistry, and cyclised using native chemical ligation. (1) Dawson's resin containing di-Fmoc-3,4-diaminobenzoic acid (Dbz) as linker is the starting point. (2) Couplings are performed using microwave-assisted Fmoc synthesis (asterisk marks the first amino acid; the last amino acid is a BOC-protected cysteine). (3) Acylation and activation of the resin bound Dbz-precursor to yield the N-acylurea peptide (Nbz-peptide). (4) Full deprotection and resin cleavage of the Nbz-peptide in one step (Ar, Aryl). Peptide cyclization (Sa) via thioesterification, (5b) S, N-intramolecular acyl shift and native chemical ligation and (Sc) oxidative folding to yield cyclotides with the native fold. Ribbon representation of a cyclotide (kalata B1, PDB ID code 1NB1) and sequence of [T20K]kalata B1 are shown. Cysteines, disulfide bonds (yellow), and intercysteine loops are indicated.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following examples which are merely illustrative and are not to be construed as a limitation of the scope of the present invention.

### Example 1: Materials and Methods

### Plant Extraction

Plant material was purchased from Alfred Galke GmbH (Bad Grund, Germany) and extracted overnight with a 1:1 (vol/vol) mixture of methanol and dichloromethane under permanent stirring at 25° C. After filtering over filter paper, 0.5 volume of water was added, and the aqueous phase was evaporated until a concentration of less than 10% methanol was reached. The extract was further applied onto a C₁₈ silica gel column (40-63 μm, Zeoprep 60; Zeochem). After washing with 100% solvent B [90% (vol/vol) acetonitrile, 0.1% (vol/vol) TFA in water] and equilibration with 100% solvent A [100% (vol/vol) water, 0.1% (vol/vol) TFA], fractions between 20% and 80% of solvent B was collected. The extract was then freeze-dried and stored at −20° C. until further use.

### RP-HPLC Fractionation, and Peptide Isolation

Crude extract or fractions were dissolved in 5% solvent B and loaded onto preparative (10 μm, 300 Å, 250 mm×21.2 mm; Phenomenex Jupiter) or semipreparative (5 μm, 100 Å, 250 mm×10 mm; Kromasil) RP C₁₈ silica gel columns equilibrated with 5% solvent B. Preparative fractionation was carried out on a Perkin Elmer Series 200 system using a gradient from 5-80% solvent B at a flow rate of 8 mL·min⁻¹ (preparative scale) or 3 mL·min⁻¹ (semipreparative scale). UV absorbance was recorded at 214 and 280 nm for analytical and preparative purposes, respectively. Caripe and Vitri peptides were purified by RP-HPLC on a Dionex Ultimate 3000 HPLC unit (Thermo-Fisher Dionex) using semipreparative (see above) and analytical (250 mm×4.6 mm) Kromasil C₁₈ columns (5 μm, 100 Å) with linear gradients of 0.1-1% min⁻¹ solvent B [90% (vol/vol) acetonitrile, 0.1% (vol/vol) TFA in water] at flow rates of 3 mL·min⁻¹ and 1 mL·min⁻¹, respectively.

### MALDI MS and Tandem MS Analysis and Peptide Identification

Analysis of the crude extract and all fractions was performed on a MALDI-TOF/TOF 4800 Analyzer (AB Sciex) operated in the reflector positive mode and acquiring 2,000-5,000 total shots per spectrum with a laser intensity set at 3,500-4,000 (arbitrary units). MS and tandem MS experiments were carried out using 10 mg·mL⁻¹ α-cyanohydroxyl cinnamic acid in 50% (vol/vol) acetonitrile as a matrix. Aliquots of each sample (0.5 μL) were mixed with 3 μL of matrix and then spotted on the plate. Spectra were acquired and processed using 4800 Analyzer software (AB Sciex). Cyclotides were then identified either by database search or by manual sequencing with the help of DataExplorer software (AB Sciex).

### Enzymatic Digestion and Peptide Sequencing

To reduce all cysteine residues within the crude extract, freshly prepared 0.2 M DTT (2 μL; Sigma Aldrich) was added to sample aliquots (20 μL) and incubated for 30 min at 60° C. in the dark. To alkylate each reduced sample, freshly prepared 0.5 M iodoacetamide (4 μL; Sigma Aldrich) was added and incubated for 10 min at 25° C. If digestion was performed, reduced and alkylated samples were then, without further purification steps, subjected to enzymatic digestion by adding 2 μL of either 0.5 μg·μL⁻¹ endoproteinase Glu-C(Sigma Aldrich) or 0.1 μg·μL⁻¹ trypsin (Sigma Aldrich) and incubated for 3 h at 37° C. The reaction was quenched by the addition of diluted TFA (1 μL). Before MS analysis, samples were desalted using C₁₈ZipTips (Millipore) and stored at 4° C.

### Cloning, Cell Culture, Transfection, and Membrane Preparation

kOR sequence was inserted into pEGFP-N1 plasmids (Clontech) using BamHI and HindIII restriction sites (to yield a C-terminal GFP fusion protein). The conditions for the propagation of HEK293 cells and creation of stably transfected cell lines were similar as described previously (Hicks, J Neuroendocrinol 24 (7), 2012, 1012-29). Cells were harvested, and membranes were prepared as described previously (Hicks, loc. cit.).

### Radioligand Binding Assays

Membranes (5-10 μg per assay) from HEK293 cells stably expressing the mouse the kOR were incubated in a final volume of 300 μL containing 50 mM Tris-HCl, 5 mM MgCl₂, 0.1% (wt/vol) BSA (pH 7.4), competing ligands and [³H]-diprenorphine (1 nM for competition binding). [³H]-diprenorphine was purchased from PerkinElmer Life Sciences. After 60 min at 37° C., the reaction was terminated by rapid filtration over glass-fiber filter mats [Skatron FilterMAT 11731 (Molecular Devices, Sunnyvale, CA)]). Nonspecific binding was determined in the presence of 10 μM naloxone (Sigma Aldrich). Specific binding represents the difference between total and nonspecific binding and is presented as normalized data. IC₅₀ values and Hill coefficients were obtained by fitting the data to a three-parameter logistic equation (Hill equation) using a Levenberg-Marquardt algorithm.

Functional cAMP Assays

The cellular cAMP levels were measured in HEK293 cells stably expressing the kOR and using Cisbio cAMP Gi Kit (62AM9PEC). Cells were centrifuged at 800 rpm, the supernatant was aspirated, and the cell pellet was resuspended in 1× stimulation buffer containing 0.5 mM IBMX. 2000 cells/well were seeded into a white 384-well plate and then treated as indicated with ligands diluted in 1× stimulation buffer followed by incubation at 37° C. for 30 min. The stimulation was terminated by sequential addition of 5 μL/well cryptate-labeled cAMP and 5 μL/well anti-cAMP d2 conjugate, each diluted (1:20) in lysis detection buffer. After a 1-hour incubation at room temperature, the time-resolved fluorescence energy transfer (TR-FRET) was measured with a lag time of 100 μs and an integration time of 300 μs using a Flexstation 3 (Molecular Devices, San Jose, USA). The resulting 620/665 nm fluorescence ratio values were plotted in GraphPad Prism 5.0 (GraphPad Software, La Jolla, CA).

### Beta Arrestin Recruitment Assay

Recruitment of β-arrestin 2 upon receptor stimulation was measured via real-time measurement of bioluminescence-resonance-energy-transfer (BRET) between 3-arrestin-luciferase and EGFP-tagged the kOR. Cells were co-transfected with β-arrestin 2-Nluc (obtained as a gift by Kevin Pfleger under a Limited Use Label License (NanoLuc, Promega, Madison, USA) and the kOR-EGFP encoding plasmids at a ratio of 1:10. At 6 h post-transfection, the cells were transferred onto a white, clear bottom 96-well plate at 50,000 cells/well in phenol-red free DMEM containing 10% fetal bovine serum. The following day, the cells were serum starved for 1 h in phenol-free DMEM. Furimazine (Promega, Madison, USA), diluted 1:50 in Hank's balanced salt solution, was added to the cells 5 min prior to monitoring at a 1:1 ratio. Light emissions were measured at 460 nm (Nluc) and 510 nm (EGFP) on a Flexstation 3 (Molecular Devices, San Jose, USA). After establishment of a baseline for 5 min, ligands diluted in HBSS were added and the response measured for 35 min. The ligand-induced BRET signal was calculated as: (emission EGFP_{ligand}/emission Nluc_{ligand})−(emission EGFP_{HBSS}/emission Nluc_{HBSS}). Concentration-response curves at the kOR were generated from the BRET signal at 5 min after addition of various ligand concentrations. The allosteric modulation of [T20K]-kalata B1 and U50,488 at the kOR was measured by co-incubation at 37° C. for 5 min.

### Peptide Conjugation

Peptide conjugation was performed similarly as previously described (Thell, loc. cit.). [T20K]-kalata B1 was dissolved in 0.1 M NaHCO₃ buffer, pH 8.5. A 20-fold molar excess of VivoTag 680 XL (PerkinElmer) was prepared in anhydrous DMSO and the reaction was allowed to proceed at 25° C. for 4 h. Reaction was stopped with 0.1% TFA. Purification of labeled peptide from excess of reagent was achieved by semipreparative HPLC using a diChrom Kromasil C₁₈ column (250×10 mm, 5 μm) and linear gradients 5 to 80% solvent B [double-distilled H₂O/CH₃CN/TFA, 10/90/0.1% (vol/vol/vol), solvent A 0.1% TFA aqueous]. HPLC fractions were analyzed via MALDI-TOF mass spectrometry in the negative reflector mode. Purity of peptide samples was determined to be ≥95% based on analytical HPLC and detection of VivoTag label in the A₂₈₀ UV trace.

### EAE and In Vivo Imaging

C57BL/6 mice were immunized at day 0 with 75 μL of equal amounts of MOG (MOG₃₅₋₅₅, 1 mg/mL; Charite Berlin) and incomplete Freud's adjuvants (Sigma-Aldrich) supplemented with 10 mg/mL Mycobacterium tuberculosis H37Ra (Difco) s.c. into the left and right flank. Additionally, mice received i.p. 200 ng pertussis toxin (Millipore,) solubilized in 100 μL PBS at day 0 and day 2. C57BL/6 mice were immunized at day 0 according to the protocol described recently in (Thell, loc. cit.). Progression of EAE was divided into five clinical stages: score 0, no signs; score 1, complete tail paralysis; score 2, partial paraparesis; score 3, severe paraparesis; score 4, tetraparesis; and score 5, moribund condition. When a mouse meets exclusion criteria (score >4, loss of weight >20%, no water and food uptake, no grooming) then it is considered moribund. Mice were euthanized by deeply anesthetizing them with ketamine reaching a score of 3-4 due to ethical guidelines. Conjugated [T20K]-kalata B1 (5 mg kg⁻¹) was injected i.p. at disease stages 0.5 and 1.75. At 4 h post-injection the organs were harvested, and the signal response was monitored by IVIS.

### Example 2: Cyclotides Isolated from Carapichea ipecacuanha and Viola tricolor are Ligands of the kOR

The kOR has recently emerged as an appealing target for developing remyelination therapies of multiple sclerosis as well as an alternative for developing safer and more effective analgesics (Du, loc. cit.; Mei, 2014, loc. cit.; Che, Cell 172 (1-2), 2018, 55-67 e15). The binding screening efforts with a plant library identified several plant extracts containing cysteine-rich peptides that bind to the kOR (FIG. 1). Among these plants, plant extracts from Psychotria solitudinum, Viola tricolor, Carapichea ipecacuanha, Viola odorata, Momordica charantia and Beta vulgaris showed the most pronounced binding effect. Given their affinity towards the kOR, it was sought to identify cysteine-rich peptides present in these extracts. It was started with the C. ipecacuanha (ipecac root) as several cysteine-rich peptides, the so-called cyclotides, have previously been isolated from this plant. The ipecac extract was initially subjected to HPLC-based purification to separate alkaloids (e.g. emetine, cephaeline) from peptides. Peptide-enriched fractions were then analyzed in radioligand binding assay and in fact, they showed the ability to bind to the kOR (FIG. 2A). Following, six cyclotides previously isolated from the ipecac root extract (Fahradpour, loc. cit.) were assayed in radioligand binding. Intriguingly, all six cyclotides were capable of binding to the kOR (FIG. 2B). Caripe 10 was then used to generate a concentration-response curve. When compared to the dynorphin A 1-13—the endogenous the kOR peptide ligand—caripe 10 displaced tritiated diprenorphine in a concentration-dependent manner with a Kᵢ value of 1 μM (FIG. 2C). As cyclotides from the ipecac root extract acted as ligands of the kOR, the cyclotide-rich plant extract form V. tricolor was further subjected to bioactivity-guided fractionation. Peptide-enriched fractions exhibited the affinity towards the kOR whereby the fraction nine was the most active one (FIG. 2D). This peptide-enriched fraction was further purified to isolate cyclotides responsible for the binding affinity. Intriguingly, a novel cyclotide was isolated and sequenced from the most active fraction (FIG. 2E) and it was subsequently demonstrated that this novel vitri cyclotide is able to bind to the kOR (FIG. 2F). Overall, these data provide evidence that cyclotides isolated from C. ipecacuanha and V. tricolor bind to the kOR with an affinity in a low μM range.

### Example 3: [T20K]-Kalata B1 Binds to and Activates the kOR

The ability of cyclotides isolated from C. ipecacuanha and V. tricolor to bind to the kOR, prompted us to pharmacologically characterize [T20K]-kalata B1 in radioligand binding and functional studies. The binding and functional data revealed that the [T20K]-kalata B1 binds to and activates the kOR with a Kᵢ of 2.7 μM and an EC₅₀ of 17 μM, respectively (FIGS. 3A and B). Dynorphin A 1-13 was used as a positive control showing a Kᵢ value of 280 μM and an EC₅₀ of 14 nM. the kOR is widely expressed throughout the central nervous system (CNS). Selective activation of the kOR produces anti-nociception in animal models without a risk of physical dependence or respiratory failure. However, it is notable that, in addition to the analgesic effect, the kOR activation has been shown to have undesirable side effects including dysphoria and sedation (Darcq, Nat Rev Neurosci 19 (8), 2018, 499-514). These side effects might dampen the therapeutic potential of the kOR agonists in the treatment of demyelinating diseases. Intriguingly, beta arrestins, cytosolic proteins that regulate GPCR signaling, have been linked to development of side effects associated with the activation of opioid receptors (Darcq, loc. cit.). Thus, it was set out to examine the ability of the [T20K]-kalata B1 to induce beta arrestin recruitment in a BRET-based assay. Consequently, when [T20K]-kalata B1 was incubated with HEK293 cells transiently co-expressing NanoLuc-β-arrestin 2 and EGFP—the kOR no recruitment of beta arrestin could be detected (FIGS. 3C and D). By contrast, dynorphin A 1-13 was able to recruit beta arrestin with an EC₅₀ of 183 nM. These data suggest that [T20K]-kalata B1 is a full agonist of the kOR and is less likely to develop centrally-mediated the kOR side effects.

### Example 4: [T20K]-Kalata B1 is an Allosteric Modulator of the kOR

Over the past few years, the concept of allosteric modulation has gained scientific momentum in the field of GPCRs (Conn, Nat Rev Drug Discov 8 (1), 2009, 41-54]. Several allosteric modulators which bind to a receptor site distinct from the orthosteric site have been identified as a novel approach of the treatment of CNS disorders (Conn, loc. cit.). Compounds that possess an allosteric mode of action can display a variety of theoretical advantages over orthosteric ligands as potential therapeutic agents. For example, allosteric modulators that do not display any agonism are quiescent in the absence of endogenous orthosteric activity and only exert their effect in the presence of a released orthosteric agonist (Conn, loc. cit.). Thus, such allosteric modulators have the potential to maintain activity dependence and both temporal and spatial aspects of endogenous physiological signaling. A second potential advantage of allosteric ligands is greater receptor selectivity due to either higher sequence divergence in allosteric sites across receptor subtypes relative to the conserved orthosteric domain, or due to selective cooperativity at a given subtype to the exclusion of others (Conn, loc. cit.). Alternatively, selectivity might be engendered by combining both orthosteric and allosteric pharmacophores within the same molecule to yield a novel class of ‘bitopic’ GPCR ligands (Conn, loc. cit.; Valant, Annu Rev Pharmacol Toxicol 52, 2012, 153-78). Accordingly, using radioligand and functional assays it was sought to investigate the allosteric effect of [T20K]-kalata B1 at the kOR. Radioligand binding studies were conducted in HEK293 cells stably expressing the kOR and co-incubating varying concentrations of [T20K]-kalata B1 with either dynorphin A 1-13 or U50,488, a selective the kOR agonist. Herein, it was revealed that the [T20K]-kalata B1 negatively modulates tritiated diprenorphine and only slightly affects the affinity of the endogenous dynorphin A 1-13 (FIG. 4A, Table 1). Similar effect was provoked when [T20K]-kalata B1 was co-incubated with U50,488 (FIG. 3B, Table 2). Surprisingly, when functional studies were carried out by measuring cellular cAMP levels, it was observed that [T20K]-kalata B1 does not affect the potency of the dynorphin A 1-13 but instead leads to an increase in its efficacy (FIG. 4C, Table 1). However, the co-incubation with U50,488 led to a reverse allosteric effect, i.e. [T20K]-kalata B1 induced a nine-fold leftward shift in the potency and it only slightly affected the efficacy of the orthosteric ligand (FIG. 4D, Table 2). This observation was in agreement of a classic example of the probe dependence of the allosteric interactions at the kOR. As evident, the change in the direction and/or magnitude of an allosteric modulation greatly depends on the nature of the orthosteric probe ligand. Given that U50,488 is capable of inducing beta arrestin recruitment, [T20K]-kalata B1 was further co-incubated with U50,488 to check if [T20K]-kalata B1 has an impact on the ability of U50,488 to recruit beta arrestin. As a matter of fact, co-stimulation of the kOR with [T20K]-kalata B1 resulted in a remarkable alleviation of the efficacy of the U50,488 thereby not impinging on its potency (FIGS. 4E and F, Table 2).

These data show that [T20K]-kalata B1 is not only an orthosteric ligand at the kOR but also acts as a bitopic ligand capable of engaging the kOR in an allosteric manner. Thus, this phenomenon of an allosteric modulation of [T20K]-kalata B1 at the kOR may exert additionally beneficial effects in the treatment of MS.

### Example 5: [T20K]-Kalata B1 Accumulates in the Brain in an EAE Model of MS

To consider [T20K]-kalata B1 and the kOR as candidates for the treatment of MS, one has to probe the ability of [T20K]-kalata B1 to cross the blood-brain barrier (BBB). For this purpose, the state-of-the-art in vivo model for MS was used, the murine EAE assay. Prior to injection, [T20K]-kalata B1 was conjugated to VivoTag 680 XL Fluorochrome followed by its HPLC-based purification as previously described (Thell, loc. cit.). Once the mice developed disease, conjugated [T20K]-kalata B1 was intraperitoneally injected during different disease stages (0.5 and 1.75) and 4h post-injection organs were harvested and biodistribution has been monitored using the IVIS. The labeled cyclotide accumulated in the brain and also in the spine but to a far less extent (FIGS. 5A, C and D). The EvansBlue dye was used as a positive control and it showed strong signal in the brain and spine (FIG. 5B, E, F).

Finally, these data confirm the ability of [T20K]-kalata B1 to penetrate the CNS in the EAE model, thereby making [T20K]-kalata B1 eminently suitable for further investigations regarding MS treatment in a combination with U50,488.

### Example 6: Ex Vivo/In Vivo Treatment of EAE Mice by [T20K]-Kalata B1 in Combination with U50,488/Dynorphin a 1-13 Results in an Increased Remyelination/Decreased Demyelination and Increased EAE Clinical Score

The kOR has been identified as a promising target to promote oligodendrocyte differentiation and myelination. Oligodendrocytes are one of the supporting glial cells of the CNS that form myelin. Myelin is an essential component of the CNS and supports electrical conduction and metabolic support to the underlying axon. In neurodegenerative conditions, such as multiple sclerosis (MS), the myelin sheath is being damaged. The adult brain contains a population of stem cell-like, oligodendrocyte progenitor cells (OPCs) that can differentiate into new oligodendrocytes then remyelinate the exposed axons thereby displaying an endogenous repair process.

The KOR agonist nalfurafine and U50,488 have been shown to be active both in vitro and in vivo (Denny, loc. cit.; Du loc. cit.). Therefore, a study is designed that enables a comparison between, for example, T20K and these ligands on the promotion of OPC differentiation and remyelination, as well as their improved effectiveness in combination.

### 1. Ex Vivo Study Protocol:

Rat neonate brains are dissected, and cerebellum and meninges are removed. Brains are dissociated via enzyme digestion for 1 h, after which digestion is stopped by adding culture medium (Dulbecco's modified eagle medium, DMEM), 10% foetal bovine serum (FBS), 1% penicillin-streptomycin (Pen/Strep) to the cells. Dissociated cells are centrifuged and supernatant removed. The cells are suspended in culture medium by trituration with 18- and 23-gauge needles and added to T75 flasks at approx. 1.5 cortices per flask. Cells are cultured for 10 days with media changes occurring every 2-3 days. At Day 10, loosely adherent microglia are removed by shaking the flasks at 250 rpm for 1 h. Flasks are then replenished with media and shaken overnight for 18.20 h at 230 rpm.

On the following day, media containing the OPCs are removed from each flask. The collected cells are plated on non-treated TC petri dishes for 20 min to remove any contaminating astrocytes by their differential adhesion. The remaining purified OPCs are centrifuged, counted and plated at 7,000 cells per well onto poly-D-lysine coated 96-well plates in SATO media (DMEM, 5 mL 100×SATO, 5 mL Pen/Strep, 5 mL insulin, transferrin, selenite (ITS) supplement) containing 10 ng/mL of growth factors PDGFAA and FGF (Day 0). OPCs are maintained in culture for 48 h prior to initiating the assay. On Day 2, media is removed and fresh media (containing test substances (T20K and/or U50,488 and/or nalfurafine) or suitable reference substances (such as T3, 3,3′,5-Triiod-L-thyronin) added. The time period of incubation of these substances is optimized. The original protocol would recommend incubation for 72 h, but this may lead to non-conclusive results due to cytotoxicity (since these primary neuronal cells are very sensitive to xenobiotika, such as T20K). Therefore, the incubation protocol is optimized for shorter time periods (e.g. 30 min, 1 h, 4 h, 8 h or 24 h). Subsequently, cells are fixed with PFA (4%, 10 min) and antibodies against Olig2 (such as Merck; MABN50), NG2 (Merck; AB5320) and MBP (Biorad; MCA409S) are administered. Plates are imaged using a suitable high-content imaging system and cell counts performed, quantifying the number of cells positive for each marker. Low power phase contrast images are taken on a regular basis, to highlight the morphological changes observed during culture. After immunostaining, wells are imaged at 10× magnification. Images are processed using automated image analysis, to calculate cell counts. Five fields of view are imaged from each well, and cells positive for each marker are counted. Different concentrations of test and reference compounds are assessed; treatments are cultured with 3 technical replicates and the experiment is performed independently at least 3 times (N=3) on cells pooled from multiple rat pups.

The study is designed to quantify the remyelinating effects of T20K, alone and in combination with known KOR agonists. For this purpose, the percentage of oligodendroglia that are MBP positive is calculated for each condition, to reveal any effects of differentiation. An increase in MBP-positive oligodendroglia indicates such an effect. Olig2 is a marker for undifferentiated OPCs and NG2 is an integral membrane proteoglycan found in the plasma membrane of many diverse cell types, which provides an overall cell viability status of cells.

### 2. In Vivo Study Protocol:

In vivo the remyelination activity of T20K in combination with KOR agonists is determined in the EAE mouse model of multiple sclerosis. EAE is induced and mice are treated as previously established (Thell et al.). Degree of myelination as compared to control treatments are performed by histology. Spinal cords of mice are isolated, fixed in 4% buffered formalin, and processed for histological evaluation. Sections are stained with H&E and LFB using standard protocols. Furthermore, sections are analyzed for CD3 surface expression using immunohistochemistry with rat-anti-CD3 (e.g. AbD Serotech) and goat-anti-rat (e.g. Vector Laboratories) antibodies. A minimum of three cross-sections of each animal are evaluated histologically. Inflammatory index is calculated as follows: The number of perivascular infiltrates in spinal cord cross-sections are divided by the number of used cross-sections for each animal. Therefore, a higher inflammatory index indicates more inflammatory infiltrates. To evaluate the extent of demyelinated area, total and demyelinated area of each cross section is measured in the KLB staining. The demyelinated area will then be calculated and plotted as percent of total cross-section. For instance, Image J is used for all histological evaluations.

In addition, the degree if T-cell infiltration from the periphery into the CNS is determined, since KOR activation on peripheral T-cells is thought to be important for its CNS remyelinating activity (REF New Zealand paper). Immune cells were isolated from the CNS by digesting brain of appropriate animals with a mixture of 5 mL collagenase D (0.233 U/mg; Roche) and DNase I (Roche) (0.17 U/mL collagenase D and 0.01 mg/mL DNase I per organ). Brains are incubated for 30 min at 37° C. in a shaking incubator. For further disruption of the tissue, EDTA (pH 8.0 in PBS) is added for a final concentration of 2 mM and suspension was pipetted up and down for 5 min at 23° C. before filtering through a 70-μm cell strainer. Cells are washed with PBS at 400×g for 8 min at 4° C. before resuspension in RPMI media. Cells were used for FACS analysis or seeded at a concentration of 3×10⁶/mL and stimulated ex vivo with 30 μg/mL MOG. Supernatants of stimulated cells are used for detection of cytokine secretion using an ELISA (for details refer to Thell et al.).

Treatment of EAE diseased mice with T20K in combination with KOR agonists are expected to lead to lower inflammatory indices and reduced areas of axonal demyelination as compared with the EAE-induced control treated mice. In addition, quantification of CD3-, CD4-, or CD8-positive cells from the brain of treated mice is expected to result in a reduced number of CD3+, CD4+ cells in the CNS, and a decrease in CD3 is expected to indice in spinal cord cells.

The present invention refers to the following Tables:

TABLE 1
Binding and functional data of [T20K]
with or without dynorphin A 1-13

| | Receptor | Receptor activation | |
|---|---|---|---|
| [T20K]-kalata | binding | cAMP EC₅₀ (nM) | |
| B1 ± dynorphin | Kᵢ values | and Eₘₐₓ (%) values | |
| A 1-13 | (pM) | Eₘₐₓ (%) | EC₅₀ |
| 0 | 290 | 96 | 17 |
| 0.03 | n.d. | 113 | 10 |
| 0.1 | n.d. | 104 | 12 |
| 0.3 | 328 | 119 | 27 |
| 1 | 277 | 134 | 19 |
| 3 | 284 | 137 | 19 |
| 10 | 414 | n.d. | n.d. |
| 30 | 343 | n.d. | n.d. |

TABLE 2
Binding and functional data of [T20K] with or without U50,488

| | Receptor | Receptor activation EC₅₀ (pM) | | | |
|---|---|---|---|---|---|
| | binding | and Eₘₐₓ (%) values | | | |
| [T20K]-kalata | Kᵢ values | Eₘₐₓ (%) | EC₅₀ | Eₘₐₓ (%) | EC₅₀ |
| B1 ± U50,488 | (nM) | [cAMP] | [cAMP] | [β-arrestin] | [β-arrestin] |
| 0 | 11 | 82 | 989 | 104 | 6 |
| 0.03 | n.d. | 82 | 908 | n.d. | n.d. |
| 0.1 | n.d. | 87 | 602 | n.d. | n.d. |
| 0.3 | 15 | 94 | 532 | n.d. | n.d. |
| 1 | 16 | 84 | 502 | n.d. | n.d. |
| 3 | 19 | 104 | 411 | n.d. | n.d. |
| 10 | 15 | 99 | 104 | 58 | 7 |
| 30 | 31 | n.d. | n.d. | n.d. | n.d. |

TABLE 3
Kalata-type cyclotides

| | | | Monoi- | |
|---|---|---|---|---|
| | | | sotopic | |
| Name | Sequence | Class | Mass | Organism |
| [N29K] | GLPVCGETCVGGTCNT | Cyclo- | 2904.19 | Synthetic |
| kalata | PGCTCSWPVCTRK | tide | | |
| B1 | | | | |
| [V10K] | GLPVCGETCKGGTCNT | Cyclo- | 2919.17 | Synthetic |
| kalata | PGCTCSWPVCTRN | tide | | |
| B1 | | | | |
| [T20K] | GLPVCGETCVGGTCNT | Cyclo- | 2917.19 | Synthetic |
| kalata | PGCKCSWPVCTRN | tide | | |
| B1 | | | | |
| kalata | GFPCGESCVYVPCLTA | Cyclo- | 3091.28 | Oldenlandia |
| B19 | AIGCSCSNKVCYKN | tide | | affinis |
| [N29A] | GLPVCGETCVGGTCNT | Cyclo- | 2847.14 | Synthetic |
| kalata | PGCTCSWPVCTRA | tide | | |
| B1 | | | | |
| [R28A] | GLPVCGETCVGGTCNT | Cyclo- | 2805.08 | Synthetic |
| kalata | PGCTCSWPVCTAN | tide | | |
| B1 | | | | |
| [T27A] | GLPVCGETCVGGTCNT | Cyclo- | 2860.13 | Synthetic |
| kalata | PGCTCSWPVCARN | tide | | |
| B1 | | | | |
| [V25A] | GLPVCGETCVGGTCNT | Cyclo- | 2862.11 | Synthetic |
| kalata | PGCTCSWPACTRN | tide | | |
| B1 | | | | |
| [P24A] | GLPVCGETCVGGTCNT | Cyclo- | 2864.13 | Synthetic |
| kalata | PGCTCSWAVCTRN | tide | | |
| B1 | | | | |
| [W23A] | GLPVCGETCVGGTCNT | Cyclo- | 2775.1 | Synthetic |
| kalata | PGCTCSAPVCTRN | tide | | |
| B1 | | | | |
| [S22A] | GLPVCGETCVGGTCNT | Cyclo- | 2874.15 | Synthetic |
| kalata | PGCTCAWPVCTRN | tide | | |
| B1 | | | | |
| [T20A] | GLPVCGETCVGGTCNT | Cyclo- | 2860.13 | Synthetic |
| kalata | PGCACSWPVCTRN | tide | | |
| B1 | | | | |
| [G18A] | GLPVCGETCVGGTCNT | Cyclo- | 2904.16 | Synthetic |
| kalata | PACTCSWPVCTRN | tide | | |
| B1 | | | | |
| [P17A] | GLPVCGETCVGGTCNT | Cyclo- | 2864.13 | Synthetic |
| kalata | AGCTCSWPVCTRN | tide | | |
| B1 | | | | |
| [T16A] | GLPVCGETCVGGTCNA | Cyclo- | 2860.13 | Synthetic |
| kalata | PGCTCSWPVCTRN | tide | | |
| B1 | | | | |
| [N15A] | GLPVCGETCVGGTCAT | Cyclo- | 2847.14 | Synthetic |
| kalata | PGCTCSWPVCTRN | tide | | |
| B1 | | | | |
| [T13A] | GLPVCGETCVGGACNT | Cyclo- | 2860.13 | Synthetic |
| kalata | PGCTCSWPVCTRN | tide | | |
| B1 | | | | |
| [G12A] | GLPVCGETCVGATCNT | Cyclo- | 2904.16 | Synthetic |
| kalata | PGCTCSWPVCTRN | tide | | |
| B1 | | | | |
| [G11A] | GLPVCGETCVAGTCNT | Cyclo- | 2904.16 | Synthetic |
| kalata | PGCTCSWPVCTRN | tide | | |
| B1 | | | | |
| [V10A] | GLPVCGETCAGGTCNT | Cyclo- | 2862.11 | Synthetic |
| kalata | PGCTCSWPVCTRN | tide | | |
| B1 | | | | |
| [T8A] | GLPVCGEACVGGTCNT | Cyclo- | 2860.13 | Synthetic |
| kalata | PGCTCSWPVCTRN | tide | | |
| B1 | | | | |
| [E7A] | GLPVCGATCVGGTCNT | Cyclo- | 2832.14 | Synthetic |
| kalata | PGCTCSWPVCTRN | tide | | |
| B1 | | | | |
| [G6A] | GLPVCAETCVGGTCNT | Cyclo- | 2904.16 | Synthetic |
| kalata | PGCTCSWPVCTRN | tide | | |
| B1 | | | | |
| [V4A] | GLPACGETCVGGTCNT | Cyclo- | 2862.11 | Synthetic |
| kalata | PGCTCSWPVCTRN | tide | | |
| B1 | | | | |
| [P3A] | GLAVCGETCVGGTCNT | Cyclo- | 2864.13 | Synthetic |
| kalata | PGCTCSWPVCTRN | tide | | |
| B1 | | | | |
| [L2A] | GAPVCGETCVGGTCNT | Cyclo- | 2848.1 | Synthetic |
| kalata | PGCTCSWPVCTRN | tide | | |
| B1 | | | | |
| [G1A] | ALPVCGETCVGGTCNT | Cyclo- | 2904.16 | Synthetic |
| kalata | PGCTCSWPVCTRN | tide | | |
| B1 | | | | |
| kalata | GVPCAESCVYIPCIST | Cyclo- | 3143.31 | Oldenlandia |
| B18 | VLGCSCSNQVCYRN | tide | | affinis |
| kalata | GIPCAESCVYIPCTIT | Cyclo- | 3183.4 | Oldenlandia |
| B17 | ALLGCKCKDQVCYN | tide | | affinis |
| kalata | GIPCAESCVYIPCTIT | Cyclo- | 3184.39 | Oldenlandia |
| B16 | ALLGCKCQDKVCYD | tide | | affinis |
| kalata | GLPVCGESCFGGSCYT | Cyclo- | 2974.13 | Oldenlandia |
| B15 | PGCSCTWPICTRD | tide | | affinis |
| kalata | GLPVCGESCFGGTCNT | Cyclo- | 3020.15 | Oldenlandia |
| B14 | PGCACDPWPVCTRD | tide | | affinis |
| kalata | GLPVCGETCFGGTCNT | Cyclo- | 3034.16 | Oldenlandia |
| B13 | PGCACDPWPVCTRD | tide | | affinis |
| kalata | GSLCGDTCFVLGCNDS | Cyclo- | 2878.08 | Oldenlandia |
| B12 | SCSCNYPICVKD | tide | | affinis |
| kalata | GLPVCGETCFGGTCNT | Cyclo- | 2882.09 | Oldenlandia |
| B11 | PGCSCTDPICTRD | tide | | affinis |
| kalata | GLPTCGETCFGGTCNT | Cyclo- | 3028.14 | Oldenlandia |
| B10 | PGCSCSSWPICTRD | tide | | affinis |
| oia | | | | |
| kalata | GLPTCGETCFGGTCNT | Cyclo- | 3046.16 | Oldenlandia |
| B10 | PGCSCSSWPICTRD | tide | | affinis |
| linear | | | | |
| kalata | GLPTCGETCFGGTCNT | Cyclo- | 3028.14 | Oldenlandia |
| B10 | PGCSCSSWPICTRD | tide | | affinis |
| kalata | GSVFNCGETCVLGTCY | Cyclo- | 3288.32 | Oldenlandia |
| B9 | TPGCTCNTYRVCTKD | tide | | affinis |
| linear | | | | |
| kalata | GSVFNCGETCVLGTCY | Cyclo- | 3270.3 | Oldenlandia |
| B9 | TPGCTCNTYRVCTKD | tide | | affinis |
| kalata | GLPVCGETCFGGTCNT | Cyclo- | 2953.14 | Oldenlandia |
| B2 | PGCSCTWPICTRD | tide | | affinis |
| kyn | | | | |
| kalata | GLPVCGETCFGGTCNT | Cyclo- | 2953.14 | Oldenlandia |
| B2 | PGCSCTWPICTRD | tide | | affinis |
| nfk | | | | |
| kalata | GLPVCGETCVGGTCNT | Cyclo- | 2890.14 | Oldenlandia |
| B1 | PGCTCSWPVCTRN | tide | | affinis |
| nfk | | | | |
| kalata | GLPVCGETCVGGTCNT | Cyclo- | 2890.14 | Oldenlandia |
| B1 | PGCTCSWPVCTRN | tide | | affinis |
| oia | | | | |
| [W19K, | GLPVCGETCVGGTCNT | Cyclo- | 2878.17 | Synthetic |
| P20N, | PGCTCSKNKCTRN | tide | | |
| V21K]- | | | | |
| kalata | | | | |
| B1 | | | | |
| [P20D, | GLPVCGETCVGGTCNT | Cyclo- | 2937.14 | Synthetic |
| V21K]- | PGCTCSWDKCTRN | tide | | |
| kalata | | | | |
| B1 | | | | |
| kalata | GSVLNCGETCLLGTCY | Cyclo- | 3281.37 | Oldenlandia |
| B8 | TTGCTCNKYRVCTKD | tide | | affinis |
| kalata | GLPVCGETCFGGTCNT | Cyclo- | 2953.14 | Oldenlandia |
| B2 | PGCSCTWPICTRD | tide | | affinis |
| | | | | Viola |
| | | | | odorata |
| kalata | GLPVCGETCVGGTCNT | Cyclo- | 2890.14 | Oldenlandia |
| B1 | PGCTCSWPVCTRN | tide | | affinis |
| | | | | Viola |
| | | | | tricolor |
| | | | | Viola |
| | | | | baoshanensis |
| | | | | Viola |
| | | | | yedoensis |
| | | | | Viola |
| | | | | philippica |
| kalata | GTPCGESCVYIPCISG | Cyclo- | 3059.27 | Oldenlandia |
| B5 | VIGCSCTDKVCYLN | tide | | affinis |
| kalata | GLPVCGETCVGGTCNT | Cyclo- | 2890.14 | Synthetic |
| B1 | PGCTCSWPVCTRN | tide | | Viola |
| IIa | | | | odorata |
| | | | | Oldenlandia |
| | | | | affinis |
| | | | | Viola |
| | | | | tricolor |
| | | | | Viola |
| | | | | arvensis |
| kalata | GLPVCGETCVGGTCNT | Cyclo- | 2876.13 | Viola |
| S | PGCSCSWPVCTRN | tide | | baoshanensis |
| | | | | Viola |
| | | | | yedoensis |
| | | | | Viola |
| | | | | biflora |
| | | | | Viola |
| | | | | philippica |
| | | | | Viola |
| | | | | ignobilis |
| kalata | GLPVCGETCVGGTCNT | Cyclo- | 2891.13 | Oldenlandia |
| B4 | PGCTCSWPVCTRD | tide | | affinis |
| kalata | GLPVCGETCTLGTCYT | Cyclo- | 3069.27 | Oldenlandia |
| B7 | QGCTCSWPICKRN | tide | | affinis |
| kalata | GLPTCGETCFGGTCNT | Cyclo- | 3080.17 | Oldenlandia |
| B3 | PGCTCDPWPICTRD | tide | | affinis |
| kalata | GLPTCGETCFGGTCNT | Cyclo- | 3027.15 | Oldenlandia |
| B6 | PGCSCSSWPICTRN | tide | | affinis |
| kalata | RNGLPVCGETCVGGTC | Cyclo- | 2908.16 | Synthetic |
| b1-6b | NTPGCTCSWPVCT | tide | | |
| kalata | VCGETCVGGTCNTPGC | Cyclo- | 2370.86 | Synthetic |
| b1-6a | TCSWPVCT | tide | | |
| kalata | VCTRNGLPVCGETCVG | Cyclo- | 2625.03 | Synthetic |
| b1-5 | GTCNTPGCTCS | tide | | |
| kalata | CSWPVCTRNGLPVCGE | Cyclo- | 2807.11 | Synthetic |
| b1-4 | TCVGGTCNTPGC | tide | | |
| kalata | GCTCSWPVCTRNGLPV | Cyclo- | 2710.06 | Synthetic |
| b1-3 | CGETCVGGTCN | tide | | |
| kalata | GTCNTPGCTCSWPVCT | Cyclo- | 2908.16 | Synthetic |
| b1-2 | RNGLPVCGETCVG | tide | | |
| kalata | TCVGGTCNTPGCTCSW | Cyclo- | 2722.1 | Synthetic |
| b1-1 | PVCTRNLPVCG | tide | | |

TABLE 4
Caripe-type cyclotides

| | | | Monoi- | |
|---|---|---|---|---|
| | | | sotopic | |
| Name | Sequence | Class | Mass | Organism |
| caripe | GIPCGESCVFIPCFTS | Cyclo- | 3237.37 | Carapichea |
| 13 | VFGCSCKDKVCYRN | tide | | ipecacuanha |
| caripe | GVIPCGESCVFIPCFS | Cyclo- | 3287.45 | Carapichea |
| 12 | SVIGCSCKNKVCYRN | tide | | ipecacuanha |
| caripe | GVIPCGESCVFIPCIS | Cyclo- | 3281.53 | Carapichea |
| 11 | TVIGCSCKKKVCYRN | tide | | ipecacuanha |
| caripe | GVIPCGESCVFIPCFS | Cyclo- | 3301.47 | Carapichea |
| 10 | TVIGCSCKNKVCYRN | tide | | ipecacuanha |
| caripe | XCVFIPCTITALLGCS | Cyclo- | 2542.11 | Carapichea |
| 9 | CSNNVCYKN | tide | | ipecacuanha |
| caripe | GVIPCGESCVFIPCIT | Cyclo- | 3237.51 | Carapichea |
| 8 | AAIGCSCKKKVCYRN | tide | | ipecacuanha |
| caripe | GIPCGESCVFIPCTVT | Cyclo- | 3253.47 | Carapichea |
| 7 | ALLGCSCKNKVCYRN | tide | | ipecacuanha |
| caripe | GAICTGTCFRNPCLSR | Cyclo- | 3199.4 | Carapichea |
| 6 | RCTCRHYICYLN | tide | | ipecacuanha |
| caripe | XCGESCVFIPCFTSVF | Cyclo- | 2970.21 | Carapichea |
| 5 | GCSCKDKVCYRN | tide | | ipecacuanha |
| caripe | LICSSTCLRIPCLSPR | Cyclo- | 3080.42 | Carapichea |
| 4 | CTCRHHICYLN | tide | | ipecacuanha |
| caripe | GIPCGESCVFIPCISA | Cyclo- | 3041.27 | Carapichea |
| 3 | VVGCSCSNKVCYNN | tide | | ipecacuanha |
| caripe | GIPCGESCVFIRCTIT | Cyclo- | 3243.41 | Carapichea |
| 2 | ALLGCSCSNNVCYKN | tide | | ipecacuanha |
| caripe | GVIPCGESCVFIPCIS | Cyclo- | 3268.47 | Carapichea |
| 1 | TVIGCSCKDKVCYRN | tide | | ipecacuanha |

TABLE 5
Viola-type cyclotides

| | | | Monoi- | |
|---|---|---|---|---|
| | | | sotopic | |
| Name | Sequence | Class | Mass | Organism |
| viba 32 | GLPVCGEACVGGTCNTPGCSCSWPVCTRN | Cyclotide | 2846.12 | Viola tricolor |
| viba 30 linear | GPPVCGETCVGGTCNTPGCSCSWPVCTRN | Cyclotide | 2878.12 | Viola tricolor |
| vitri peptide 18b | GSVFNCGETCVFGTCFTSGCSCVYRVCSKD | Cyclotide | 3134.22 | Viola tricolor |
| vitri peptide 50 | GDIPCGESCVYIPCITGVLGCSCSHNVCYYN | Cyclotide | 3244.29 | Viola tricolor |
| vitri peptide 24a | GGTIFNCGESCFQGTCYTKGCACGDWKLCY | Cyclotide | 3490.33 | Viola tricolor |
| | GEN | | | |
| vitri peptide 39 | GAPICGESCFTGTCYTVQCSCSWPVCTRN | Cyclotide | 3066.2 | Viola tricolor |
| linear | | | | |
| vitri peptide 39 | GAPICGESCFTGTCYTVQCSCSWPVCTRN | Cyclotide | 3048.18 | Viola tricolor |
| vitri peptide 38 | GDTCYETCFTGFCFIGGCKCDFPVCVKN | Cyclotide | 3038.22 | Viola tricolor |
| vitri peptide | GGTIFSCGESCFQGTCYTKGCACGDWKLCY | Cyclotide | 3463.32 | Viola tricolor |
| 36/37 | GEN | | | |
| vitri peptide 30 | GFACGETCIFTSCFITGCTCNSSLCFRN | Cyclotide | 2960.15 | Viola tricolor |
| vitri peptide 29 | GVPSSDCLETCFGGKCNAHRCTCSQWPLCAKN | Cyclotide | 3390.39 | Viola tricolor |
| vitri peptide 27a | GAFTPCGETCLTGECHTEGCSCVGQTFCVKK | Cyclotide | 3171.27 | Viola tricolor |
| vitri peptide | GEPVCGDSCVFFGCDDEGCTCGPWSLCYRN | Cyclotide | 3194.14 | Viola tricolor |
| 24/28 | | | | |
| vitri peptide 23 | GLPTCGETCTLGTCYTPGCTCSWPLCTKN | Cyclotide | 2985.19 | Viola tricolor |
| vitri peptide 94b | GVAVCGETCTLGTCYTPGCSCDWPICKRN | Cyclotide | 3012.22 | Viola tricolor |
| vitri peptide 22a | GAPVCGETCFTGLCYSSGCSCIYPVCNRN | Cyclotide | 2997.18 | Viola tricolor |
| linear | | | | |
| vitri peptide 22a | GAPVCGETCFTGLCYSSGCSCIYPVCNRN | Cyclotide | 2979.16 | Viola tricolor |
| vitri peptide 21 | GGPLDCQETCTLSDRCYTKGCTCNWPICYKN | Cyclotide | 3447.39 | Viola tricolor |
| vitri peptide 20 | GDLVPCGESCVYIPCLTTVLGCSCSENVCYRN | Cyclotide | 3372.41 | Viola tricolor |
| vitri peptide 18a | GVPICGETCFQGTCNTPGCTCKWPICERN | Cyclotide | 3092.25 | Viola tricolor |
| vitri peptide 17 | GSDDQVACGESCAMTPCFMHVVGCVCSQKVCYR | Cyclotide | 3488.37 | Viola tricolor |
| vitri peptide 14 | GSSCGETCEVFSCFITRCACIDGLCYRN | Cyclotide | 3012.18 | Viola tricolor |
| vitri peptide | GTIFDCGETCLLGKCYTPGCSCGSWALCYGQN | Cyclotide | 3325.31 | Viola tricolor |
| 9a/53 | | | | |
| vitri peptide 8 | PTPCGETCIWISCVTAAIGCYCHESICYR | Cyclotide | 3172.31 | Viola tricolor |
| vitri peptide 4 | GTPCGESCIYVPCISAVFGCWCQSKVCYKD | Cyclotide | 3221.32 | Viola tricolor |
| vitri peptide 3 | GSWPCGESCVYIPCITSIAGCECSKNVCYKN | Cyclotide | 3295.39 | Viola tricolor |
| vitri peptide 2 | GSIPCGESCVWIPCISGIAGCSCSNKVCYLN | Cyclotide | 3138.32 | Viola tricolor |
| vitri peptide 1 | GLIPCGESCVWIPCISSVIGCSCKSKVCYKN | Cyclotide | 3251.48 | Viola tricolor |
| VocC | GLPVCGETCVGGTCNTPGCSCSWPVCIRN | Cyclotide | 2888.16 | Viola odorata |
| vigno 10 | GTIPCGESCVWIPCISSVVGCSCKSKVCYKD | Cyclotide | 3226.41 | Viola tricolor |
| | | | | Viola ignobilis |
| vigno 9 | GIPCGESCVWIPCISSALGCSCKSKVCYRN | Cyclotide | 3138.37 | Viola tricolor |
| | | | | Viola ignobilis |
| vigno 7 | GTLPCGESCVWIPCISSVVGCSCKNKVCYKN | Cyclotide | 3252.44 | Viola tricolor |
| | | | | Viola ignobilis |
| vigno 6 | GIPCGESCVWIPCISSAIGCSCKGSKVCYRN | Cyclotide | 3195.39 | Viola tricolor |
| | | | | Viola ignobilis |
| vigno 5 | GLPLCGETCVGGTCNTPGCSCGWPVCVRN | Cyclotide | 2858.15 | Viola tricolor |
| | | | | Viola ignobilis |
| vigno 4 | GLPLCGETCVGGTCNTPACSCSWPVCTRN | Cyclotide | 2904.16 | Viola tricolor |
| | | | | Viola ignobilis |
| vigno 3 | GLPLCGETCVGGTCNTPGCSCSWPVCTRN | Cyclotide | 2890.14 | Viola tricolor |
| | | | | Viola ignobilis |
| vitri F | GTLPCGESCVWIPCISSVVGCACKSKVCYKD | Cyclotide | 3210.41 | Viola tricolor |
| vitri E | GLPVCGETCVGGTCNTPGCSCSWPVCFRN | Cyclotide | 2922.15 | Viola odorata |
| | | | | Viola tricolor |
| vitri D | GLPVCGETCFTGSCYTPGCSCNWPVCNRN | Cyclotide | 3043.16 | Viola tricolor |
| vitri C | GLPICGETCVGGTCNTPGCFCTWPVCTRN | Cyclotide | 2964.19 | Viola tricolor |
| vitri B | GYPICGESCVGGTCNTPGCSCSWPVCTTN | Cyclotide | 2871.05 | Viola tricolor |
| vaby C | GLPVCGETCAGGRCNTPGCSCSWPVCTRN | Cyclotide | 2903.15 | Viola tricolor |
| | | | | Viola |
| | | | | abyssinica |
| cycloviolacin O36 | GLPTCGETCFGGTCNTPGCTCDPFPVCTHD | Cyclotide | 3008.1 | Viola odorata |
| cycloviolacin O27 | GSIPACGESCFKGWCYTPGCSCSKYPLCAKD | Cyclotide | 3236.3 | Viola odorata |
| cycloviolacin O26 | GSIPACGESCFRGKCYTPGCSCSKYPLCAKD | Cyclotide | 3206.32 | Viola odorata |
| cycloviolacin O30 | GIPCGESCVWIPCISSAIGCSCKNKVCFKN | Cyclotide | 3121.38 | Viola odorata |
| cycloviolacin O29 | GIPCGESCVWIPCISGAIGCSCKSKVCYKN | Cyclotide | 3080.35 | Viola odorata |
| cycloviolacin O35 | GLPVCGETCVGGTCNTPYCFCSWPVCTRD | Cyclotide | 3043.19 | Viola odorata |
| cycloviolacin O34 | GLPVCGETCVGGTCNTEYCTCSWPVCTRD | Cyclotide | 3029.16 | Viola odorata |
| cycloviolacin O33 | GLPVCGETCVGGTCNTPYCTCSWPVCTRD | Cyclotide | 2997.17 | Viola odorata |
| cycloviolacin O32 | GAPVCGETCFGGTCNTPGCTCDPWPVCTND | Cyclotide | 2980.07 | Viola odorata |
| cycloviolacin O28 | GLPVCGETCVGGTCNTPGCSCSWPVCFRD | Cyclotide | 2923.13 | Viola odorata |
| | | | | Viola tricolor |
| cycloviolacin O31 | GLPVCGETCVGGTCNTPGCSCSIPVCTRN | Cyclotide | 2803.13 | Viola odorata |
| | | | | Viola tricolor |
| Viba 11 | GIPCGESCVWIPCISGAIGCSCKSKVCYRN | Cyclotide | 3108.36 | Viola |
| | | | | baoshanensis |
| | | | | Viola |
| | | | | philippica |
| Viba 9 | GIPCGESCVWIPCISSAIGCSCKNKVCYRK | Cyclotide | 3179.43 | Viola tricolor |
| | | | | Viola |
| | | | | baoshanensis |
| Mra30 | GIPCGESCVFIPCLTSAIGCSCKSKVCYRN | Cyclotide | 3113.37 | Viola tricolor |
| | | | | Melicytus |
| | | | | ramiflorus |
| | | | | Viola |
| | | | | philippica |
| Viba 15 | GLPVCGETCVGGTCNTPGCACSWPVCTRN | Cyclotide | 2860.13 | Viola tricolor |
| | | | | Viola |
| | | | | baoshanensis |
| | | | | Viola |
| | | | | philippica |
| vibi G | GTFPCGESCVFIPCLTSAIGCSCKSKVCYKN | Cyclotide | 3220.4 | Viola tricolor |
| | | | | Viola biflora |
| | | | | Psychotria |
| | | | | leptothyrsa |
| vibi C | GLPVCGETCAFGSCYTPGCSCSWPVCTRN | Cyclotide | 2973.15 | Viola tricolor |
| | | | | Viola biflora |
| cycloviolacin O25 | DIFCGETCAFIPCITHVPGTCSCKSKVCYFN | Cyclotide | 3361.42 | Viola odorata |
| cycloviolacin O24 | GLPTCGETCFGGTCNTPGCTCDPWPVCTHN | Cyclotide | 3046.13 | Viola odorata |
| cycloviolacin O23 | GLPTCGETCFGGTCNTPGCTCDSSWPICTHN | Cyclotide | 3137.15 | Viola odorata |
| cycloviolacin O22 | GLPICGETCVGGTCNTPGCTCSWPVCTRN | Cyclotide | 2904.16 | Viola odorata |
| | | | | Viola tricolor |
| | | | | Palicourea |
| | | | | tetragona |
| cycloviolacin O21 | GLPVCGETCVTGSCYTPGCTCSWPVCTRN | Cyclotide | 2969.17 | Viola odorata |
| cycloviolacin O20 | GIPCGESCVWIPCLTSAIGCSCKSKVCYRD | Cyclotide | 3153.37 | Viola odorata |
| ″vitri″ cyclotide | GDPIPCGETCFTGKCYSETIGCTCEWPICTKN | | | Viola tricolor |
| (vitri peptide | | | | |
| 100) | | | | |
| cycloviolacin O19 | GTLPCGESCVWIPCISSVVGCSCKSKVCYKD | Cyclotide | 3226.41 | Viola odorata |
| cycloviolacin O18 | GIPCGESCVYIPCTVTALAGCKCKSKVCYN | Cyclotide | 3085.37 | Viola odorata |
| cycloviolacin O17 | GIPCGESCVWIPCISAAIGCSCKNKVCYRN | Cyclotide | 3149.38 | Viola odorata |
| cycloviolacin O16 | GLPCGETCFTGKCYTPGCSCSYPICKKIN | Cyclotide | 3048.28 | Viola odorata |
| cycloviolacin O15 | GLVPCGETCFTGKCYTPGCSCSYPICKKN | Cyclotide | 3034.26 | Viola odorata |
| cycloviolacin O14 | GSIPACGESCFKGKCYTPGCSCSKYPLCAKN | Cyclotide | 3177.33 | Viola odorata |
| violacin A | SAISCGETCFKFKCYTPRCSCSYPVCK | Cyclotide | 3004.26 | Viola odorata |
| | | | | Psychotria |
| | | | | leptothyrsa |
| cycloviolacin O13 | GIPCGESCVWIPCISAAIGCSCKSKVCYRN | Cyclotide | 3122.37 | Viola odorata |
| tricyclon B | GGTIFDCGESCFLGTCYTKGCSCGEWKLCYGEN | Cyclotide | 3506.35 | Viola tricolor |
| tricyclon A | GGTIFDCGESCFLGTCYTKGCSCGEWKLCYGTN | Cyclotide | 3478.35 | Viola tricolor |
| | | | | Viola arvensis |
| cycloviolacin O1 | GIPCAESCVYIPCTVTALLGCSCSNRVCYN | Cyclotide | 3114.32 | Viola odorata |
| | | | | Viola odorata |
| | | | | Oldenlandia |
| | | | | affinis |
| kalata B1 | GLPVCGETCVGGTCNTPGCTCSWPVCTRN | Cyclotide | 2890.14 | Viola tricolor |
| | | | | Viola |
| | | | | baoshanensis |
| | | | | Viola yedoensis |
| | | | | Viola |
| | | | | philippica |
| varv peptide H | GLPVCGETCFGGTCNTPGCSCETWPVCSRN | Cyclotide | 3053.17 | Viola tricolor |
| | | | | Viola arvensis |
| varv peptide G | GVPVCGETCFGGTCNTPGCSCDPWPVCSRN | Cyclotide | 3021.14 | Viola tricolor |
| | | | | Viola arvensis |
| varv peptide F | GVPICGETCTLGTCYTAGCSCSWPVCTRN | Cyclotide | 2957.17 | Viola tricolor |
| | | | | Viola arvensis |
| varv peptide D | GLPICGETCVGGSCNTPGCSCSWPVCTRN | Cyclotide | 2876.13 | Viola tricolor |
| | | | | Viola arvensis |
| varv peptide C | GVPICGETCVGGTCNTPGCSCSWPVCTRN | Cyclotide | 2876.13 | Viola tricolor |
| | | | | Viola arvensis |
| varv peptide B | GLPVCGETCFGGTCNTPGCSCDPWPMCSRN | Cyclotide | 3067.13 | Viola tricolor |
| | | | | Viola arvensis |
| cycloviolacin O10 | GIPCGESCVYIPCLTSAVGCSCKSKVCYRN | Cyclotide | 3115.35 | Viola odorata |
| cycloviolacin O7 | SIPCGESCVWIPCTITALAGCKCKSKVCYN | Cyclotide | 3152.41 | Viola odorata |
| cycloviolacin O6 | GTLPCGESCVWIPCISAAVGCSCKSKVCYKN | Cyclotide | 3181.4 | Viola odorata |
| cycloviolacin O5 | GTPCGESCVWIPCISSAVGCSCKNKVCYKN | Cyclotide | 3111.32 | Viola odorata |
| cycloviolacin O3 | GIPCGESCVWIPCLTSAIGCSCKSKVCYRN | Cyclotide | 3152.38 | Viola odorata |
| cycloviolacin O4 | GIPCGESCVWIPCISSAIGCSCKNKVCYRN | Cyclotide | 3165.38 | Viola odorata |
| | | | | Viola tricolor |
| | | | | Pombalia |
| | | | | calceolaria |
| vodo N | GLPVCGETCTLGKCYTAGCSCSWPVCYRN | Cyclotide | 3046.24 | Viola odorata |
| | | | | Viola tricolor |
| cycloviolacin O12 | GLPICGETCVGGTCNTPGCSCSWPVCTRN | Cyclotide | 2890.14 | Viola tricolor |
| | | | | Viola arvensis |
| | | | | Viola |
| | | | | baoshanensis |
| | | | | Viola yedoensis |
| | | | | Viola |
| | | | | tianshanica |
| | | | | Viola |
| | | | | abyssinica |
| | | | | Viola |
| | | | | philippica |
| | | | | Viola odorata |
| | | | | Oldenlandia |
| | | | | affinis |
| kalata S | GLPVCGETCVGGTCNTPGCSCSWPVCTRN | Cyclotide | 2876.13 | Viola tricolor |
| | | | | Viola arvensis |
| | | | | Viola |
| | | | | baoshanensis |
| | | | | Viola yedoensis |
| | | | | Viola biflora |
| | | | | Viola |
| | | | | philippica |
| | | | | Viola ignobilis |
| vitri A | GIPCGESCVWIPCITSAIGCSCKSKVCYRN | Cyclotide | 3152.38 | Viola tricolor |
| | | | | Viola biflora |
| | | | | Psychotria |
| | | | | leptothyrsa |
| cycloviolacin O9 | GIPCGESCVWIPCLTSAVGCSCKSKVCYRN | Cyclotide | 3138.37 | Viola odorata |
| | | | | Viola biflora |
| vodo M | GAPICGESCFTGKCYTVQCSCSWPVCTRN | Cyclotide | 3075.23 | Viola odorata |
| | | | | Viola tricolor |
| cycloviolacin O11 | GTLPCGESCVWIPCISAVVGCSCKSKVCYKN | Cyclotide | 3209.43 | Viola odorata |
| cycloviolacin O8 | GTLPCGESCVWIPCISSVVGCSCKSKVCYKN | Cyclotide | 3225.42 | Viola odorata |
| | | | | Viola adunca |
| cycloviolacin O2 | GIPCGESCVWIPCISSAIGCSCKSKVCYRN | Cyclotide | 3138.37 | Viola odorata |
| | | | | Viola biflora |
| | | | | Viola |
| | | | | philippica |

TABLE 6
the kOR ligands/agonists

| Description | Ligand type | Mode of action | |
|---|---|---|---|
| dynorphin A-(1-13) | Peptide | Agonist | endogenous |
| dynorphin-(1-11) | Peptide | Agonist | endogenous |
| dynorphin A | Peptide | Agonist | endogenous |
| dynorphin B | Peptide | Partial agonist | endogenous |
| dynorphin A-(1-8) | Peptide | Agonist | endogenous |
| α-neoendorphin | Peptide | Agonist | endogenous |
| β-neoendorphin | Peptide | Agonist | endogenous |
| β-endorphin | Peptide | Partial agonist | endogenous |
| E2078 | Peptide | Agonist | |
| DAMGO | Peptide | Partial agonist | |
| difelikefalin | Peptide | Agonist | |
| DN-9 | Peptide | Agonist | |
| endomorphin-1-Amo2 | Peptide | Partial agonist | |
| biphalin 5 | Peptide | Agonist | |
| CR665 | Peptide | Agonist | |
| JT09 | Peptide | Agonist | |
| ethyketazocine | Small molecule or natural product | Agonist | |
| enadoline | Small molecule or natural product | Agonist | |
| (-)-bremazocine | Small molecule or natural product | Partial agonist | |
| ethylketocyclazocine | Small molecule or natural product | Agonist | |
| (-)-cyclazocine | Small molecule or natural product | Partial agonist | |
| butorphanol | Small molecule or natural product | Partial agonist | |
| etorphine | Small molecule or natural product | Agonist | |
| GR 89696 | Small molecule or natural product | Agonist | |
| enadoline | Small molecule or natural product | Agonist | |
| U69593 | Small molecule or natural product | Agonist | |
| naloxone benzoylhydrazone | Small molecule or natural product | Partial agonist | |
| MP1104 | Small molecule or natural product | Agonist | |
| tifluadom | Small molecule or natural product | Agonist | |
| U50,488 | Small molecule or natural product | Agonist | |
| cebranopadol | Small molecule or natural product | Agonist | |
| hydromorphone | Small molecule or natural product | Agonist | |
| nalorphine | Small molecule or natural product | Partial agonist | |
| salvinorin A | Small molecule or natural product | Agonist | |
| BU08028 | Small molecule or natural product | Agonist | |
| compound 3 [HS6666; PMID: 23134120] | Small molecule or natural product | Partial agonist | |
| (-)-pentazocine | Small molecule or natural product | Partial agonist | |
| tramadol | Small molecule or natural product | Agonist | |
| normorphine | Small molecule or natural product | Agonist | |
| ADL5747 | Small molecule or natural product | Agonist | |
| nalbuphine | Small molecule or natural product | Agonist | |
| ADL5859 | Small molecule or natural product | Agonist | |
| morphine | Small molecule or natural product | Partial agonist | |
| dihydromorphine | Small molecule or natural product | Partial agonist | |
| fentanyl | Small molecule or natural product | Partial agonist | |
| etonitazene | Small molecule or natural product | Partial agonist | |
| BW373U86 | Small molecule or natural product | Agonist | |
| SCH221510 | Small molecule or natural product | Agonist | |
| UFP-512 | Small molecule or natural product | Agonist | |
| hydrocodone | Small molecule or natural product | Agonist | |
| (-)-methadone | Small molecule or natural product | Partial agonist | |
| SR16835 | Small molecule or natural product | Agonist | |
| bilorphin | Small molecule or natural product | Agonist | |
| pethidine | Small molecule or natural product | Agonist | |
| AR-M1000390 | Small molecule or natural product | Agonist | |
| asimadoline | Small molecule or natural product | Agonist | |
| spiradoline | Small molecule or natural product | Agonist | |
| ICI 204448 | Small molecule or natural product | Agonist | |
| carfentanil | Small molecule or natural product | Agonist | |
| EMD 60400 | Small molecule or natural product | Agonist | |
| TRK-820 | Small molecule or natural product | Agonist | |
| MB-1C-OH | Small molecule or natural product | Agonist | |
| SLL-039 | Small molecule or natural product | Agonist | |
| Dmt-Tiq 4a | Small molecule or natural product | Agonist | |
| Dmt-Tiq 4b | Small molecule or natural product | Agonist | |
| Dmt-Tiq 4c | Small molecule or natural product | Agonist | |
| Dmt-Tiq 4d | Small molecule or natural product | Agonist | |
| compound 3; cyclorphan | Small molecule or natural product | Agonist | |
| compound 4; 17-(Cyclopropylmethyl)-6,7-didehydro-4,5r-epoxy-13,14- | Small molecule or natural product | Agonist | |
| dihydroxy-4′-phenylindolo[2′,3′:6,7]-morphinan | | | |
| compound 5; 17-(Cyclopropylmethyl)-6,7-didehydro-4,5r-epoxy-13,14- | Small molecule or natural product | Agonist | |
| dihydroxy-4′- phenoxyindolo[2′,3′:6,7]morphinan | | | |
| compound 6; 4′-(Benzyloxy)-17-(cyclopropylmethyl)-6,7- | Small molecule or natural product | Agonist | |
| didehydro4,5r-epoxy-13,14-dihydroxyindolo[2′,3′:6,7]morphinan | | | |
| compound 7; 17-(Cyclopropylmethyl)-6,7-didehydro-4,5r-epoxy-13,- | Small molecule or natural product | Agonist | |
| 14-dihydroxy-5′-phenylindolo[2′,3′:6,7]morphinan | | | |
| compound 8; 17-(Cyclopropylmethyl)-6,7-didehydro-4,5r-epoxy-13,- | Small molecule or natural product | Agonist | |
| 14-dihydroxy-5′-phenoxyindolo[2′,3′:6,7]morphinan | | | |
| compound 9; 5′-(Benzyloxy)-17-(cyclopropylmethyl)-6,7- | Small molecule or natural product | Agonist | |
| didehydro4,5r-epoxy-13,14-dihydroxyindolo[2′,3′:6,7]morphinan | | | |
| compound 10; 17-(Cyclopropylmethyl)-6,7-didehydro4,5r-epoxy- | Small molecule or natural product | Agonist | |
| 13,14-dihydroxy-6′-phenylindolo[2′,3′:6,7]-morphinan | | | |
| compound 11; 17-(Cyclopropylmethyl)-6,7-didehydro-4,5r-epoxy- | Small molecule or natural product | Agonist | |
| 13,14-dihydroxy6′-phenoxyindolo[2′,3′:6,7]morphinan | | | |
| compound 12; 6′-(Benzyloxy)-17-(cyclopropylmethyl)-6,7-didehydro- | Small molecule or natural product | Agonist | |
| 4,5r-epoxy-13,14-dihydroxyindolo[2′,3′:6,7]morphinan | | | |
| compound 13; 17-(Cyclopropylmethyl)-6,7-didehydro-4,5r-epoxy-13,- | Small molecule or natural product | Agonist | |
| 14-dihydroxy-7′-phenylindolo[2′,3′:6,7]morphinan | | | |
| compound 14; 17-(Cyclopropylmethyl)-6,7-didehydro-4,5r-epoxy-13,- | Small molecule or natural product | Agonist | |
| 14-dihydroxy-7′-phenoxyindolo[2′,3′:6,7]morphinan | | | |
| compound 15; 7′-(Benzyloxy)-17-(cyclopropylmethyl)-6,7- | Small molecule or natural product | Agonist | |
| didehydro4,5r-epoxy-13,14-dihydroxyindolo[2′,3′:6,7]morphinan | | | |
| compound 16; trans-17-(Cyclopropylmethyl)-6,7-didehydro-4,5r- | Small molecule or natural product | Agonist | |
| epoxy13,14-dihydroxy-5′-[2-(2- | | | |
| pyridinyl)ethenyl]indolo[2′,3′:6,7]morphinan | | | |
| compound 17; 17-Allyl-3-hydroxy-4,5α-epoxy-7,8-en-6-β-[(3′- | Small molecule or natural product | Agonist | |
| iodo) benzamido]-morphinan | | | |
| compound 18; 1-(2,4-Dichlorophenyl)-3,6,6-trimethyl-1,5,6,7- | Small molecule or natural product | Agonist | |
| tetrahydro-4H-indazol-4-one | | | |
| compound 19; 1-(2,4-Dibromophenyl)-3,6,6-trimethyl-1,5,6,7- | Small molecule or natural product | Agonist | |
| tetrahydro-4H-indazol-4-one | | | |
| compound 20; 1-(2-bromo-4-chlorophenyl)-3,6,6-trimethyl-1,5,6,7- | Small molecule or natural product | Agonist | |
| tetrahydro-4H-indazol-4-one | | | |
| compound 21; 1-(2-Bromo-4-methylphenyl)-3,6,6-trimethyl-1,5,6,7- | Small molecule or natural product | Agonist | |
| tetrahydro-4H-indazol-4-one | | | |
| compound 22; 1-(2,4-Dichlorophenyl)-3-methyl-1,5,6,7-tetrahydro- | Small molecule or natural product | Agonist | |
| 4Hindazol-4-one | | | |
| NNTA | Small molecule or natural product | Agonist | |
| amine 12 | Small molecule or natural product | Agonist | |
| triazole 14 | Small molecule or natural product | Agonist | |
| O6C-20-nor-salvinorin A | Small molecule or natural product | Agonist | |
| nalfurafine | Small molecule or natural product | Biased agonist | |
| probe 1.1 [KSC-12-192; PMID: 24187130] | Small molecule or natural product | Biased agonist | |
| HS665 | Small molecule or natural product | Biased agonist | |
| HS666 | Small molecule or natural product | Biased agonist | |
| RB-64 | Small molecule or natural product | Biased agonist | |
| Mesyl sal B | Small molecule or natural product | Biased agonist | |
| 6-GNTI | Small molecule or natural product | Biased agonist | |
| collybolide | Small molecule or natural product | Biased agonist | |
| triazole1.1 | Small molecule or natural product | Biased agonist | |
| noribogaine | Small molecule or natural product | Biased agonist | |
| compound 81; Enamine; Vendor ID: Z1176485991 | Small molecule or natural product | Biased agonist | |

The present invention refers to the following nucleotide and amino acid sequences:

The present invention refers to the following nucleotide and amino acid sequences:

SEQ ID No. 1:
Amino acid sequence of Kalata B1:
GLPVCGETCVGGTCNTPGCTCSWPVCTRN
SEQ ID No. 2:
Amino acid sequence of Kalata B2:
GLPVCGETCFGGTCNTPGCSCTWPICTRD
SEQ ID No. 3:
Reference amino acid sequence of D-Kalata B2:
all-DGLPVCGETCFGGTCNTPGCSCTWPICTRD
SEQ ID No. 4:
Amino acid sequence of Kalata G18K:
GLPVCGETCVGGTCNTPKCTCSWPVCTRN
SEQ ID No. 5:
Amino acid sequence of Kalata N29K:
GLPVCGETCVGGTCNTPGCTCSWPVCTRK
SEQ ID No. 6:
Amino acid sequence of Kalata T20K, G1K:
KLPVCGETCVGGTCNTPGCKCSWPVCTRN
SEQ ID No. 7:
Amino acid sequence of Kalata T20K:
GLPVCGETCVGGTCNTPGCKCSWPVCTRN
SEQ ID No. 8:
Amino acid sequence of Kalata T8K:
GLPVCGEKCVGGTCNTPGCTCSWPVCTRN
SEQ ID No. 9:
Amino acid sequence of Kalata V10A:
GLPVCGETCAGGTCNTPGCTCSWPVCTRN
SEQ ID No. 10:
Amino acid sequence of Kalata V10K:
GLPVCGETCKGGTCNTPGCTCSWPVCTRN
SEQ ID No. 11:
Nucleotide sequence encoding Kalata B1:
GGACTTCCAGTATGCGGTGAGACTTGTGTTGGGGGAACTTGCAACACTCCAGGCTGCACTTGCT
CCTGGCCTGTTTGCACACGCAAT
SEQ ID No. 12:
Nucleotide sequence encoding Kalata B2:
GGTCTTCCAGTATGCGGCGAGACTTGCTTTGGGGGAACTTGCAACACTCCAGGCTGCTCTTGCA
CCTGGCCTATCTGCACACGCGAT
SEQ ID No. 13:
Amino acid sequence of the Kalata B1 precursor protein. The mature Kalata
B1 domain is underlined.
P56254, Kalata-B1, Oldenlandia affinis
MAKFTVCLLLCLLLAAFVGAFGSELSDSHKTTLVNEIAEKMLQRKILDGVEATLVTDVAEKMFLRKMKAEAKTSETA
DQVFLKQLQLKGLPVCGETCVGGTCNTPGCTCSWPVCTRNGLPSLAA
SEQ ID No. 14:
Amino acid sequence of the Kalata B2 precursor protein. The three mature
Kalata B2 domains are underlined.
P58454, Kalata-B2, Oldenlandia affinis
MAKFTNCLVLSLLLAAFVGAFGAEFSEADKATLVNDIAENIQKEILGEVKTSETVLTMFLKEMQLKGLPVCGETCFG
GTCNTPGCSCTWPICTRDSLPMRAGGKTSETTLHMFLKEMQLKGLPVCGETCFGGTCNTPGCSCTWPICTRDSLPMS
AGGKTSETTLHMFLKEMQLKGLPVCGETCFGGTCNTPGCSCTWPICTRDSLPLVAA
SEQ ID No. 15:
Nucleotide sequence encoding the Kalata B1 precursor protein. The nucleotide
sequence corresponding to the mature Kalata B1 domain is underlined.
>gi|15667740|gb|AF393825.1| Oldenlandia affinis kalata B1 precursor, mRNA,
complete cds
GGCACCAGCACTTTCTTAAAATTTACTGCTTTTTCTTATTTCTTGTTCTGTGCTTGCTTCTTCCATGGCTAAGTTCA
CCGTCTGTCTCCTCCTGTGCTTGCTTCTTGCAGCATTTGTTGGGGCGTTTGGATCTGAGCTTTCTGACTCCCACAAG
ACCACCTTGGTCAATGAAATCGCTGAGAAGATGCTACAAAGAAAGATATTGGATGGAGTGGAAGCTACTTTGGTCAC
TGATGTCGCCGAGAAGATGTTCCTAAGAAAGATGAAGGCTGAAGCGAAAACTTCTGAAACCGCCGATCAGGTGTTCC
TGAAACAGTTGCAGCTCAAAGGACTTCCAGTATGCGGTGAGACTTGTGTTGGGGGAACTTGCAACACTCCAGGCTGC
ACTTGCTCCTGGCCTGTTTGCACACGCAATGGCCTTCCTAGTTTGGCCGCATAATTTGCTTGATCAAACTGCAAAAA
TGAATGAGAAGGCCGACACCAATAAAGCTATCAATGTAGTTGGTCCCTGTACTTAATTTGGTTGGCTCCAAACCATG
TGTGCTGCTCTTGTTTTTGTTTTTTCTTTTTTCTTCTCTCTTTCGGGCACTCTTCAGGACATGAAGTGATGATCAGT
ACTCTTTGCTATCATGTTTTCTGTGCACACCTTCTATTGTAGGTGTTGTTGTGATGTTGATGCCCAATTGGAATAAA
CTGTTGTCGTTGTTAAAAAAAAAAAAAAAAA
SEQ ID No. 16:
Nucleotide sequence of encoding the Kalata B2 precursor protein. The
nucleotide sequences corresponding to the three mature Kalata B2 domains
are underlined.
>gi|15667746|gb|AF393828.1| Oldenlandia affinis kalata B2 precursor,
mRNA, complete cds
GGCACCAGATACAACCCCTTTCTTATAATTTATTGCTTTTCTTATTCCTTGAAAAAGGAGAAATAATATTGGATCTT
CCATGGCTAAGTTCACCAACTGTCTCGTCCTGAGCTTGCTTCTAGCAGCATTTGTTGGGGCTTTCGGAGCTGAGTTT
TCTGAAGCCGACAAGGCCACCTTGGTCAATGATATCGCTGAGAATATCCAAAAAGAGATACTGGGCGAAGTGAAGAC
TTCTGAAACCGTCCTTACGATGTTCCTGAAAGAGATGCAGCTCAAAGGTCTTCCAGTATGCGGCGAGACTTGCTTTG
GGGGAACTTGCAACACTCCAGGCTGCTCTTGCACCTGGCCTATCTGCACACGCGATAGCCTTCCTATGAGGGCTGGA
GGAAAAACATCTGAAACCACCCTTCATATGTTCCTGAAAGAGATGCAGCTCAAGGGTCTTCCAGTTTGCGGCGAGAC
TTGCTTTGGGGGAACTTGCAACACTCCAGGCTGCTCGTGCACCTGGCCTATCTGCACACGCGATAGCCTTCCTATGA
GTGCTGGAGGAAAAACATCTGAAACCACCCTTCATATGTTCCTGAAAGAGATGCAGCTCAAGGGTCTTCCAGTTTGC
GGCGAGACTTGCTTTGGGGGAACTTGCAACACTCCAGGCTGCTCGTGCACCTGGCCTATATGCACACGTGATAGCCT
TCCTCTTGTGGCTGCATAATTTGCTTCATCAAACTGCAAAATGAATAAGAAGGGACACTAAATTAGCTATGAATTTT
GTTGGCCCTTGTGTCTGGTAATTTGGTTCCCGCCAAATTAACCATATGTATGCATTGCTCCTTTTTTCTTTCTTTTT
TTTCCCCCTCATTTGGGCACTCTTCATTACATGAAGAGATCATGACGCTTTGTTACTCTGAGCACCCCCTGTTGGTG
TTGTTCACATGTTGATGCCCATGTTGGAATAAACTCTTGTTTTTGTTACCAAAZ
AAA
SEQ ID No. 17:
Consensus amino acid sequence of active cyclotides, in particular of Kalata-
type cyclotides, (Xxx₁ is any amino acid, non-natural amino acid or peptido-
mimetic; Xxx₂ is any amino acid, non-natural amino acid or peptidomimetic
but not Lys; and Xxx₃ is any amino acid, non-natural amino acid or
peptidomimetic but not Ala or Lys):
Xxx₁-Leu-Pro-Val-Cys-Gly-Glu-Xxx₂-Cys-Xxx₃-Gly-Gly-Thr-Cys-Asn-Thr-Pro-Xxx₁-
Cys-Xxx₁-Cys-Xxx₁-Trp-Pro-Xxx₁-Cys-Thr-Arg-Xxx₁
SEQ ID No. 18:
Consensus amino acid sequence of active cyclotides, in particular of Caripe-
type cyclotides, (Xxx₁ is Val, Ala or Leu, Xxx₂ is Gly, Ser or Thr, Xxx₃ is
Glu, Gly or Ser, Xxx₄ is Ser or Thr, Xxx₅ is Val, Leu or Phe, Xxx₆ is Phe
or Arg, Xxx₇ is Ile or Asn, Xxx₈ is Pro or Arg, Xxx₉ is Ile, Phe, Thr or
Leu, Xxx₁₀ is Ser, Thr, Ile or Val, Xxx₁₁ is Thr, Ser, Ala, Arg or Pro,
Xxx₁₂ is Val, Leu or Ala, Xxx₁₃ is Ile, Leu, Phe or Val, Xxx₁₄ is Gly or
Arg, Xxx₁₅ is Ser or Thr, Xxx₁₆ is Lys, Ser or Arg, Xxx₁₇ is Asn, Asp, His
or Lys, Xxx₁₈ is Lys, Asn, His or Tyr, Xxx₁₉ is Val or Ile, Xxx₂₀ is Arg,
Leu, Lys or Asn and/or Xxx₂₁ is Asn or Asp):
Gly-Xxx₁-Ile-Pro-Cys-Xxx₂-Xxx₃-Xxx₄-Cys-Xxx₅-Xxx₆-Xxx₇-Xxx₈-Cys-Xxx₉-Xxx₁₀-
Xxx₁₁-Ala-Xxx₁₂-Xxx₁₃-Xxx₁₄-Cys-Xxx₁₅-Cys-Xxx₁₆-Xxx₁₇-Xxx₁₈-Xxx₁₉-Cys-Tyr-
Xxx₂₀-Xxx₂₁
SEQ ID No. 19:
Consensus amino acid sequence of active cyclotides, in particular of Viola-
type cyclotides (any or all of Xxx₁ to Xxx₂₀ may be any amino acid, non-
natural amino acid or peptidomimetic; preferably, any or all of Xxx₁ to
Xxx₂₀ may be (a) conservative amino acid exchange(s) of the corresponding
amino acid residue(s) of the ″vitri″ cyclotide as depicted in SEQ ID
NO. 155.):
Gly-Xxx₁-Xxx₂-Xxx₃-Cys-Gly-Glu-Xxx₄-Cys-Xxx₅-Xxx₆-Xxx₇-
Xxx₈-Cys-Xxx₉-Xxx₁₀-Xxx₁₁-Xxx₁₂-Cys-
Xxx₁₃-Cys-Xxx₁₄-Xxx₁₅-Xxx₁₆-Xxx₁₇-Cys-Xxx₁₈-Xxx₁₉-Xxx₂₀
(SEQ ID NO. 19)

1. A method of
(i) treating Multiple Sclerosis (MS);
(ii) (ii) remyelination of oligodendrocytes and/or improvement of CNS lesions;
(iii) (iii) preventing or reducing demyelination and/or CNS lesions;
(iv) treating neuropathic pain and/or pain resulting from/coming along with MS;
(iv) treating CNS lesions; and/or
(v) (vi) treating a demyelinating disease, neurological disorder and/or nerve-related disease,
said method comprising the step of administering to a subject in need thereof a pharmaceutically effective amount of
(a) a cyclotide; and
(b) a ligand of the kappa opioid receptor (kOR), wherein said ligand is not a cyclotide.

2. (canceled)

3. The method according to claim 1, wherein said demyelinating disease, neurological disorder and/or nerve-related disease is selected from the group consisting of MS, optic neuritis, Devic's disease, inflammatory demyelinating diseases, central nervous system neuropathies, myelopathies (like Tabes dorsalis), leukoencephalopathies and leukodystrophies or is selected from the group consisting of Guillain-Barre syndrome and its chronic counterpart, chronic inflammatory demyelinating polyneuropathy, anti-MAG (myelin-associated glycoprotein) peripheral neuropathy, Charcot Marie Tooth (CMT) disease, copper deficiency and progressive inflammatory neuropathy.

4. The method according to claim 1, wherein said treating, remyelination, improvement, preventing or reducing comprises, or results in, decreasing the relapse rate and/or frequency of MS episodes.

5. The method according to claim 1, wherein said treating comprises, or results in,
(i) remyelination and/or improvement of CNS lesions;
(ii) preventing or reducing demyelination and/or CNS lesions; and/or
(iii) treating neuropathic pain and/or pain resulting from/coming along with MS.

6. The method according to claim 1, wherein (a) kOR-dependent adverse effect(s), for example dysphoria, sedation, diuresis and/or hallucinations, is/are reduced/ameliorated or avoided or is/are to be reduced/ameliorated or avoided.

7. The method according to claim 1, wherein said kOR is the human kOR (hkOR).

8. The method use according to claim 1, wherein said ligand of the kOR is, or is capable of acting as, an agonist of said kOR.

9. The method according to claim 8, wherein said agonist is an unbiased agonist.

10. The method according to claim 1, wherein said ligand of the kOR is capable of inducing or increasing β-arrestin 2 recruitment (in the absence of said cyclotide).

11. The method according to claim 8, wherein said agonist is a biased agonist.

12. The method according to claim 11, wherein said ligand of the kOR does not induce or increase β-arrestin 2 recruitment (in the absence of said cyclotide).

13. The method of claim 1, wherein said cyclotide is, or is capable of acting as, a (biased) (orthosteric) agonist of said kOR.

14. The method according to claim 1, wherein said cyclotide is not capable of inducing or increasing β-arrestin 2 recruitment (in the absence of said ligand of the kOR).

15. The method according to claim 1, wherein said cyclotide is or comprises a head-to-tail cyclized peptide which cyclotide chain includes six conserved cysteine residues capable of forming three disulfide bonds arranged in a cyclic cystine-knot (CCK) motive.

16. The method according to claim 1, wherein said cyclotide is a non-grafted cyclotide.

17. The method according to claim 1, wherein said cyclotide is a kalata-type, in particular kalata B-type, cyclotide, a caripe-type cyclotide or a viola-type cyclotide.

18. The method according to any claim 1, wherein said cyclotide is a kalata B1 or a mutant of kalata B1.

19. The method according to claim 1, wherein said cyclotide is a cyclotide comprising, or consisting of, a (head-to-tail) cyclized form of an amino acid sequence as depicted in SEQ ID NO: 7, 5, 4, 6, 155 or 86.

20. The method according to claim 1, wherein said cyclotide is the T20K mutant of kalata B1 (SEQ ID NO. 1), namely the mutant cyclotide as depicted in SEQ ID NO. 7.

21. The method of claim 1, wherein said ligand of the kOR is a small molecule or a peptide ligand.

22. The method according to claim 1, wherein said ligand of the kOR is selected from the group consisting of the kOR agonists as listed in Table 6.

23. The method according to claim 1, wherein said ligand of the kOR is selected from the group consisting of U50,488 and dynorphin A-(1-13) or from the group consisting of dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil.

24. The method according to claim 1, wherein said ligand of the kOR is selected from the group consisting of nalfurafine (morphine derivative), collybolide (mushroom Collybia maculate), noribogaine (metabolite of plant iboga), B-64 (Salvinorin A derivative), triazole1.1, 6-GNTI, HS666, HS665 and mesyl salvinorin B.

25. A combination of
(a) a cyclotide as defined in claim 1; and
(b) a ligand of the kOR as defined in claim 1.

26. The method according to claim 1, wherein
(a) said cyclotide is T20K (SEQ ID NO. 7) and said ligand is selected from the group consisting of
(i) nalfurafine, collybolide, noribogaine, the Salvinorin A derivative B-64, triazole1.1, 6-GNTI, HS666, HS665 and mesyl salvinorin B; or
(ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil;
(b) said cyclotide is N29K (SEQ ID NO. 5) and said ligand is selected from the group consisting of
(i) nalfurafine, collybolide, noribogaine, the Salvinorin A derivative B-64, triazole1.1, 6-GNTI, HS666, HS665 and mesyl salvinorin B; or
(ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil;
(c) said cyclotide is G18K (SEQ ID NO. 4) and said ligand is selected from the group consisting of
(i) nalfurafine, collybolide, noribogaine, the Salvinorin A derivative B-64, triazole1.1, 6-GNTI, HS666, HS665 and mesyl salvinorin B; or
(ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil;
(d) said cyclotide is T20K/G1K (SEQ ID NO. 6) and said ligand is selected from the group consisting of
(i) nalfurafine, collybolide, noribogaine, the Salvinorin A derivative B-64, triazole1.1, 6-GNTI, HS666, HS665 and mesyl salvinorin B; or
(ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil; or
(e) said cyclotide is the vitri cyclotide (SEQ ID NO. 155) and said ligand is selected from the group consisting of
(i) nalfurafine, collybolide, noribogaine, the Salvinorin A derivative B-64, triazole1.1, 6-GNTI, HS666, HS665 and mesyl salvinorin B;
(ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil; or
(f) said cyclotide is the caripe 10 (SEQ ID NO. 86) and said ligand is selected from the group consisting of
(i) nalfurafine, collybolide, noribogaine, the Salvinorin A derivative B-64, triazole1.1, 6-GNTI, HS666, HS665 and mesyl salvinorin B; or
(ii) U50,488, dynorphin A-(1-13), dynorphin-(1-11), dynorphin A, dynorphin A-(1-8), U69593, GR 89696, spiradoline, BRL-52537, JT09, difelikefalin, dynorphin, nalbuphine, pentasozin, pethidine and sulfentanil.

27. The method according to claim 1, wherein
(a) said cyclotide is T20K (SEQ ID NO. 7) and said ligand is nalfurafine;
(b) said cyclotide is N29K (SEQ ID NO. 5) and said ligand is nalfurafine;
(c) said cyclotide is G18K (SEQ ID NO. 4) and said ligand is nalfurafine;
(d) said cyclotide is T20K/G1K (SEQ ID NO. 6) and said ligand is nalfurafine;
(e) said cyclotide is vitri (SEQ ID NO. 155) and said ligand is nalfurafine;
(f) said cyclotide is caripe 10 (SEQ ID NO. 86) and said ligand is nalfurafine;
(g) said cyclotide is T20K (SEQ ID NO. 7) and said ligand is U50,488;
(h) said cyclotide is N29K (SEQ ID NO. 5) and said ligand is U50,488;
(i) said cyclotide is G18K (SEQ ID NO. 4) and said ligand is U50,488;
(j) said cyclotide is T20K/G1K (SEQ ID NO. 6) and said ligand is U50,488;
(k) said cyclotide is vitri (SEQ ID NO. 155) and said ligand is U50,488;
(l) said cyclotide is caripe 10 (SEQ ID NO. 86) and said ligand is U50,488;
(m) said cyclotide is T20K (SEQ ID NO. 7) and said ligand is dynorphin A 1-13;
(n) said cyclotide is N29K (SEQ ID NO. 5) and said ligand is dynorphin A 1-13;
(o) said cyclotide is G18K (SEQ ID NO. 4) and said ligand is dynorphin A 1-13;
(p) said cyclotide is T20K/G1K (SEQ ID NO. 6) and said ligand is dynorphin A 1-13;
(q) said cyclotide is vitri (SEQ ID NO. 155) and said ligand is dynorphin A 1-13;
(r) said cyclotide is caripe 10 (SEQ ID NO. 86) and said ligand is dynorphin A 1-13;
(s) said cyclotide is T20K (SEQ ID NO. 7) and said ligand is difelikefalin;
(t) said cyclotide is N29K (SEQ ID NO. 5) and said ligand is difelikefalin;
(u) said cyclotide is G18K (SEQ ID NO. 4) and said ligand is difelikefalin;
(v) said cyclotide is T20K/G1K (SEQ ID NO. 6) and said ligand is difelikefalin;
(w) said cyclotide is the vitri (SEQ ID NO. 155) and said ligand is difelikefalin;
(x) said cyclotide is caripe 10 (SEQ ID NO. 86) and said ligand is difelikefalin;
(y) said cyclotide is T20K (SEQ ID NO. 7) and said ligand is nalbuphine;
(z) said cyclotide is N29K (SEQ ID NO. 5) and said ligand is nalbuphine;
(aa) said cyclotide is G18K (SEQ ID NO. 4) and said ligand is nalbuphine;
(ab) said cyclotide is T20K/G1K (SEQ ID NO. 6) and said ligand is nalbuphine;
(ac) said cyclotide is the vitri (SEQ ID NO. 155) and said ligand is nalbuphine;
(ad) said cyclotide is caripe 10 (SEQ ID NO. 86) and said ligand is nalbuphine;
(ae) said cyclotide is T20K (SEQ ID NO. 7) and said ligand is pentasozin;
(af) said cyclotide is N29K (SEQ ID NO. 5) and said ligand is pentasozin;
(ag) said cyclotide is G18K (SEQ ID NO. 4) and said ligand is pentasozin;
(ah) said cyclotide is T20K/G1K (SEQ ID NO. 6) and said ligand is pentasozin;
(ai) said cyclotide is the vitri (SEQ ID NO. 155) and said ligand is pentasozin;
(aj) said cyclotide is caripe 10 (SEQ ID NO. 86) and said ligand is pentasozin;
(ak) said cyclotide is T20K (SEQ ID NO. 7) and said ligand is pethidine;
(al) said cyclotide is N29K (SEQ ID NO. 5) and said ligand is pethidine;
(am) said cyclotide is G18K (SEQ ID NO. 4) and said ligand is pethidine;
(an) said cyclotide is T20K/G1K (SEQ ID NO. 6) and said ligand is pethidine;
(ao) said cyclotide is the vitri (SEQ ID NO. 155) and said ligand is pethidine;
(ap) said cyclotide is caripe 10 (SEQ ID NO. 86) and said ligand is pethidine;
(aq) said cyclotide is T20K (SEQ ID NO. 7) and said ligand is sulfentanil;
(ar) said cyclotide is N29K (SEQ ID NO. 5) and said ligand is sulfentanil;
(as) said cyclotide is G18K (SEQ ID NO. 4) and said ligand is sulfentanil;
(at) said cyclotide is T20K/G1K (SEQ ID NO. 6) and said ligand is sulfentanil;
(au) said cyclotide is the vitri (SEQ ID NO. 155) and said ligand is sulfentanil; and
(av) said cyclotide is caripe 10 (SEQ ID NO. 86) and said ligand is sulfentanil.

28. (canceled)

29. A method for
(i) reducing adverse effects of a ligand of kOR; and/or
(ii) increasing the efficacy of a ligand of kOR
the method comprising administering a cyclotide.

30-31. (canceled)

32. A kit (kit of contents/kit of parts) comprising in two different vials,
(a) a cyclotide as defined in claim 1; and
(b) a ligand of the kOR as defined in claim 1 or a combination of (a) and (b).

33-36. (canceled)

37. A method of producing the combination according to claim 25, said method comprising the step of mixing the cyclotide and the kOR ligand; and optionally the further step of admixing a pharmaceutically acceptable carrier.
